(19) United States
(12) Patent Application Publication
(10) Pub. No.: US 2014/0045763 A1
(43) Pub. Date: Feb. 13, 2014

(54) COMPOUNDS FOR TREATING, DELAYING AND/OR PREVENTING A HUMAN GENETIC DISORDER SUCH AS MYOTONIC DYSTROPHY TYPE I (DMI)

(71) Applicant: PROSENSA TECHNOLOGIES B.V., LEIDEN (NL)

(72) Inventors: Maria Begoña Aguilera Diez, Leiden (NL); Peter Christian de Visser, Leiden (NL); Susan Allegonda Maria Mulders, Groesbeek (NL)

(73) Assignee: PROSENSA TECHNOLOGIES B.V., LEIDEN (CH)

(21) Appl. No.: 14/056,464

(22) Filed: Oct. 17, 2013

Related U.S. Application Data
(63) Continuation of application No. PCT/NL2012/050273, filed on Apr. 23, 2012.
(60) Provisional application No. 61/478,096, filed on Apr. 22, 2011.

(30) Foreign Application Priority Data
Apr. 22, 2011 (EP) 11163581.9

(51) Int. Cl. C07K 19/00

(52) U.S. Cl. CPC C07K 19/00
USPC 514/17.7; 530/322; 435/375

(57) ABSTRACT

The current invention provides new compounds for treating, delaying and/or preventing a human genetic disorder such as myotonic dystrophy type 1 (DM1), spino-cerebellar ataxia 8 and/or Huntington's disease-like 2 caused by expansions of CUG repeats in the transcripts of DM1/DMPK, SCA8 or JPH3 genes.

### FIELD OF THE INVENTION

The current invention provides new compounds for treating, delaying and/or preventing a human genetic disorder such as DM1.

### BACKGROUND OF THE INVENTION

Myotonic dystrophy type 1 (DM1) is a dominantly inherited neuromuscular disorder with a complex, multisystemic pathology (Harper P. S. et al). DM1 is characterized by expression of DMPK transcripts comprising long CUG repeats, which sequester or upregulate splice and transcription factors, thereby interfering with normal cellular function and viability. Antisense oligonucleotide (AON) mediated suppression of toxic DMPK transcripts is considered a potential therapeutic strategy for this frequent trinucleotide repeat disorder. The CUG repeat is present in exon 15 of the DMPK transcript.

The (CUG)ₙ tract itself forms an obvious target, being the only known polymorphism between mutant and normal-sized transcripts. In a previous study, we identified a 2′-β-methyl phosphorothioate-modified (CAG)₇ oligonucleotide (PS58) (SEQ ID NO:1) that is capable of inducing breakdown of mutant transcripts in DM1 cell and animal models (Mulders S. A. et al). For AONs to be clinically effective in DM1, they need to reach a wide variety of tissues, and cell types therein, and be successfully delivered into the nuclei of these cells. In the current invention, new compounds have been designed based on PS58 and comprising a methylated cytosine and/or an abasic site as explained herein, said compounds have an improved activity, targeting and/or delivering to and/or uptake by multiple tissues including heart, skeletal and smooth muscle.

WO 2009/099326 and WO 2007/808532 describe oligomers comprising a (CAG)ₙ repeat unit, such as PS58.

### DETAILED DESCRIPTION OF THE INVENTION

In a first aspect, there is provided a compound comprising or consisting of LGAQSNF/(NAG)ₘ in which N, as comprised in the oligonucleotide part (NAG)ₘ is C (i.e. cytosine) or 5-methylcytosine. Such a compound may be called a conjugate. This compound comprises a peptide part comprising or consisting of LGAQSNF (SEQ ID NO:2) which is linked to or coupled to or conjugated with an oligonucleotide part comprising or consisting of (NAG)ₘ in which N is C or 5-methylcytosine. This compound could also be named a conjugate. The slash (/) in LGAQSNF/(NAG)ₘ designates the linkage, coupling or conjugation between the peptide part and the oligonucleotide part of the compound according to the invention. The peptide part of the compound of the invention comprises or consists of LGAQSNF. The oligonucleotide part of the compound of the invention comprises or consists of (NAG)ₘ in which N is C or 5-methylcytosine. In an embodiment, the compound comprising or consisting of LGAQSNF/(NAG)ₘ in which N, as comprised in the oligonucleotide part (NAG)ₘ is C or 5-methylcytosine is such that at least one occurrence of A, as comprised in the oligonucleotide part (NAG), comprises a 2,6-diaminopurine nucleobase modification. The m is preferably an integer which is 4, 5, 6, 7, 8, 9, 10, 11, 12, 13, 14, 15, 16, 17, 18, 19, 20, 21, 22, 23, 24, 25, 26, 27, 28, 29 or 30. In a preferred embodiment, m is 7. Accordingly, a preferred (NAG)ₘ in which N is C or 5-methylcytosine has a length from 12 to 90 nucleotides, more preferably 12 to 45 nucleotides, even more preferably 15 to 36 nucleotides, most preferably 21 nucleotides. Said oligonucleotide part preferably comprises at least 15 to 45 consecutive nucleotides complementary to a repeat sequence CUG, or at least 18 to 42 consecutive nucleotides complementary to a repeat sequence CUG, more preferably 21 to 36 nucleotides, even more preferably 18 to 24 nucleotides, complementary to a repeat sequence CUG.

The compound according to this aspect of the invention may consist of LGAQSNF/(NAG)ₘ, which means that no other amino acids are present apart from the LGAQSNF sequence and no other nucleotides are present apart from the repeating NAG motif. Alternatively, the compound can comprise LGAQSNF/(NAG)ₘ, which means that other amino acids, or analogues or equivalents thereof, may be present apart from the LGAQSNF sequence and/or other nucleotides, or analogues or equivalents thereof, may be present at one or at both sides of the repeating NAG motif.

In the context of the present invention, an “analogue” or an “equivalent” of an amino acid is to be understood as an amino acid which comprises at least one modification with respect to the amino acids which occur naturally in peptides. Such a modification may be a backbone modification and/or a sugar modification and/or a base modification, which is further explained and exemplified below.

In the context of the present invention, an “analogue” or an “equivalent” of a nucleotide is to be understood as a nucleotide which comprises at least one modification with respect to the nucleotides which occur naturally in RNA, such as A, C, G and U. Such a modification may be a backbone modification and/or a sugar modification and/or a base modification, which is further explained and exemplified below.

In a preferred embodiment, the oligonucleotide part according to this aspect of the invention can be represented by L-(X)ₚ—(NAG)ₘ-(Y)_{q}-L, wherein N and m are as defined above. Each occurrence of L is, individually, a hydrogen atom or the linkage part, coupling part or conjugation part, as defined further below, connected to or associated with the peptide part of the compound according to the invention, wherein at least one occurrence of L is the linkage part, coupling part or conjugation part. In a preferred embodiment, one occurrence of L is a hydrogen atom and the other occurrence of L is the linkage part, coupling part or conjugation part. In another embodiment, both occurrences of L are hydrogen, and the oligonucleotide is linked, coupled or conjugated to the peptide part via one of the internal nucleotides, such as via a nucleobase or via an internucleoside linkage. Each occurrence of X and Y is, individually, an abasic site as defined further below or a nucleotide, such as A, C, G, U or an analogue or equivalent thereof and p and q are each individually an integer, preferably 0, 1, 2, 3, 4, 5, 6, 7, 8, 9, 10, or higher than 10 or up to 50. Thus, p and q are each individually an integer from 0 to 50, preferably an integer from 0 to 10, more preferably from 0 to 6. Thus, when p is 0, X is absent and when q is 0, Y is absent.

Herein, (X)ₚ—(NAG)ₘ-(Y)_{q}, wherein N and m are as defined above and p and q are 0, is regarded the oligonucleotide part of a compound according to this aspect of the invention, wherein its oligonucleotide part consists of (NAG)ₘ. Such an oligonucleotide part comprising (NAG)ₘ can be represented by (X)ₚ—(NAG)ₘ-(Y)_{q}, wherein N, m, X, Y, p and q are as defined above and at least one of p and q is not 0.

In a preferred embodiment, p is not 0, and (X)ₚ is represented by (X′)_{p′}AG or (X′)_{p″}G, wherein each occurrence of X′ is, individually, an abasic site or a nucleotide, such as A, C, G, U or an analogue or equivalent thereof, and p′ is p−2 and p″ is p−1. Such compound may be represented as:

L-(X′)_{p′}AG-(NAG)ₘ(Y)_{q}-L or

L-(X′)_{p″}-G-(NAG)ₘ-(Y)_{q}-L.

In an equally preferred embodiment, q is not 0, and (Y)_{q} is represented by NA(Y′)_{q′} or N(Y′)_{q″}, wherein N is as defined above and each occurrence of Y′ is, individually, an abasic site or a nucleotide, such as A, C, G, U or an analogue or equivalent thereof, and q′ is q−2 and q″ is q−1. Such compound may be represented as:

L-(X)ₚ—(NAG)ₘ-NA(Y′)_{q′}-L or

L-(X)ₚ—(NAG)ₘ-N(Y′)_{q″}-L.

In another preferred embodiment, both p and q are not 0, and both (X)ₚ and (Y)_{q} are represented by (X′)_{p′}AG or (X′)_{p″}G and NA(Y′)_{q′} or N(Y′)_{q″} respectively, wherein N, X′, Y′, p′, p″, q′ and q″ are as defined above. Such compound may be represented as:

L-(X′)_{p′}AG-(NAG)ₘ-NA(Y′)_{q′}-L,

L-(X′)_{p″}G-(NAG)ₘ-NA(Y′)_{q′}-L,

L-(X′)_{p′}AG-(NAG)ₘ-N(Y′)_{q″}-L, or

L-(X′)_{p″}G-(NAG)ₘ-N(Y′)_{q″}-L.

It is to be understood that p′, p″, q′ and q″ may not be negative integers. Thus, when (X)ₚ is represented by (X′)_{p′}AG or (X′)_{p″}G, p is at least 1 or at least 2 respectively, and when (Y)_{q} is represented by NA(Y′)_{q}′ or N(Y′)_{q′}, q is at least 1 or at least 2 respectively.

The oligonucleotide part of the compound according to this aspect of the invention can therefore comprise or consist of one of the following sequences: (NAG)ₘ, AG(NAG)ₘ, G(NAG)ₘ, AG(NAG)ₘNA, G(NAG)ₘNA, (NAG)ₘNA, AG(NAG)ₘN, G(NAG)ₘN, or (NAG)ₘN. In an embodiment, one or more free termini of the oligonucleotide part, i.e. the terminus where L is hydrogen, may contain 1 to 10 abasic sites, as defined further below. These abasic sites may be of the same or different types and connected through 3′-5′,5′-3′,3′-3′ or 5′-5′ linkages between each other and with the oligonucleotide part. Although technically 3′ and 5′ atoms are not present in abasic sites (because of absence of the nucleobase and thus numbering of atoms that ring), for clarity reasons these are numbered as they are in the corresponding nucleotides.

In a second aspect, the invention relates to a compound comprising or consisting of the oligonucleotide sequence (NAG)ₘ, in which N is C or 5-methylcytosine and wherein at least one occurrence of N is 5-methylcytosine and/or at least one occurrence of A comprises a 2,6-diaminopurine nucleobase modification. In a preferred embodiment, all occurrences of N are 5-methylcytosine. In another preferred embodiment, all occurrences of A comprise a 2,6-diaminopurine nucleobase. In another preferred embodiment, all occurrences of N are 5-methylcytosine and all occurrences of A comprise a 2,6-diaminopurine nucleobase. In a further preferred embodiment, the compound according to this aspect of the invention does not comprise a hypoxanthine base or, in other words, an inosine nucleotide.

The m is preferably an integer, which is preferably 4, 5, 6, 7, 8, 9, 10, 11, 12, 13, 14, 15. In other words, m is preferably 4-15, more preferably 5-12, and even more preferably 6-8. In an especially preferred embodiment, m is 5, 6, 7. The oligonucleotide comprising (NAG)ₘ may have a length of 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 nucleotides. In other words, the oligonucleotide according to this aspect of the invention preferably has a length of 12 to 90 nucleotides, more preferably 15 to 49 nucleotides, even more preferably 21 nucleotides. Said oligonucleotide preferably comprises at least 15 to 45 consecutive nucleotides complementary to a repeat sequence CUG, or at least 18 to 42 consecutive nucleotides complementary to a repeat sequence CUG, more preferably 18 to 36 nucleotides, even more preferably 18 to 24 nucleotides, complementary to a repeat sequence CUG.

The compound according to this aspect of the invention can be regarded as an oligonucleotide. Such an oligonucleotide can consist of (NAG)ₘ, which means that no other nucleotides are present, apart from the repeating NAG motif. Alternatively, the oligonucleotide can comprise (NAG)ₘ, which means that at one or at both sides of the repeating NAG motif other nucleotides, or analogues or equivalents thereof, are present.

In the context of the present invention, an “analogue” or an “equivalent” of a nucleotide is to be understood as a nucleotide which comprises at least one modification with respect to the nucleotides which occur naturally in RNA, such as A, C, G and U. Such a modification may be a backbone modification and/or a sugar modification and/or a base modification, which is further explained and exemplified below.

Alternatively, the oligonucleotide according to this aspect of the invention can be represented by H—(X)ₚ—(NAG)ₘ-(Y)_{q}—H, wherein N and m are as defined above. Each occurrence of X and Y is, individually, an abasic site as defined further below or a nucleotide, such as A, C, G, U or an analogue or equivalent thereof and p and q are each individually an integer, preferably 0, 1, 2, 3, 4, 5, 6, 7, 8, 9, 10, or higher than 10 or up to 50. Thus, p and q are each individually an integer from 0 to 50, preferably an integer from 0 to 10, more preferably from 0 to 6. Thus, when p is 0, X is absent and when q is 0, Y is absent. The skilled person will appreciate that an oligonucleotide will always start with and end with a hydrogen atom (H), regardless of the amount and nature of the nucleotides present in the oligonucleotide.

Herein, H—(X)ₚ—(NAG)ₘ-(Y)_{q}—H, wherein N and m are as defined above and p and q are 0, is regarded a compound according to this aspect of the invention which consists of (NAG)ₘ. A compound comprising (NAG)ₘ can be represented by H—(X)ₚ—(NAG)ₘ-(Y)_{q}—H, wherein N, m, X, Y, p and q are as defined above and at least one of p and q is not 0.

In a preferred embodiment, p is not 0, and (X)ₚ is represented by (X′)_{p′}AG or (X′)_{p″}G, wherein each occurrence of X′ is, individually, an abasic site or a nucleotide, such as A, C, G, U or an analogue or equivalent thereof, and p′ is p−2 and p″ is p−1. Such oligonucleotides may be represented as:

H—(X′)_{p′}AG-(NAG)ₘ-(Y)_{q}—H or

H—(X′)_{p″}G-(NAG)ₘ-(Y)_{q}—H.

In an equally preferred embodiment, q is not 0, and (Y)_{q} is represented by NA(Y′)_{q′} or N(Y′)_{q″}, wherein N is as defined above and each occurrence of Y′ is, individually, an abasic site or a nucleotide, such as A, C, G, U or an analogue or equivalent thereof, and q′ is q−2 and q″ is q−1. Such oligonucleotides may be represented as:

H—(X)ₚ—(NAG)ₘ-NA(Y′)_{q′}—H or

H—(X)ₚ—(NAG)ₘ-N(Y′)_{q″}—H.

In another preferred embodiment, both p and q are not 0, and both (X)ₚ and (Y)_{q} are represented by (X′)_{p′}AG or (X′)_{p″}G and NA(Y′)_{q′} or N(Y′)_{q″} respectively, wherein N, X′, Y′, p′, p″, q′ and q″ are as defined above. Such oligonucleotides may be represented as:

H—(X′)_{p′AG—(NAG)}ₘ-NA(Y′)_{q′}—H,

H—(X′)_{p″}G—(NAG)ₘ-NA(Y′)_{q′}—H,

H—(X′)_{p′}AG—(NAG)ₘ-N(Y′)_{q″}—H, or

H—(X′)_{p″}G—(NAG)ₘ-N(Y′)_{q″}—H.

It is to be understood that p′, p″, q′ and q″ may not be negative integers. Thus, when (X)ₚ is represented by (X′)_{p′}AG or (X′)_{p″}G, q is at least 1 or at least 2 respectively, and when (Y)_{q} is represented by NA(Y′)_{q′} or N(Y′)_{q″}, q is at least 1 or at least 2 respectively.

The oligonucleotide according to this aspect of the invention can therefore comprise or consist of one of the following sequences: (NAG)ₘ, AG(NAG)ₘ, G(NAG)ₘ, AG(NAG)ₘNA, G(NAG)ₘNA, (NAG)ₘNA, AG(NAG)ₘN, G(NAG)ₘN, or (NAG)ₘN. In an embodiment, one or more free termini of the oligonucleotide may contain 1 to 10 abasic sites, as defined further below. These abasic sites may be of the same or different types and connected through 3′-5′,5′-3′,3′-3′ or 5′-5′ linkages between each other and with the oligonucleotide. Although technically 3′ and 5′ atoms are not present in abasic sites (because of absence of the nucleobase and thus numbering of atoms that ring), for clarity reasons these are numbered as they are in the corresponding nucleotides.

Whenever (X)ₚ and/or (Y)_{q} comprises one or more abasic sites, this abasic site may be present at one or both of the termini of the oligonucleotide. Thus, at the 5′-terminus and/or at the 3′-terminus of the oligonucleotide according to this aspect of the invention, one or more abasic sites may be present. However, abasic sites may also be present within the oligonucleotide sequence, as is discussed further below.

An especially preferred oligonucleotide according to the invention is represented by H—(X)ₚ—(NAG)ₘ-(Y)_{q}—H, wherein m=5, 6, 7 and all occurrences of N are 5-methylcytosine.

An especially preferred oligonucleotide according to the invention is represented by H—(X)ₚ—(NAG)ₘ-(Y)_{q}—H, wherein m=5, 6, 7, all occurrences of N are 5-methylcytosine, p=q=0 and X and Y are absent.

Another especially preferred oligonucleotide according to the invention is represented by H—(X)ₚ—(NAG)ₘ-(Y)_{q}—H, wherein m=5, 6, 7, all occurrences of N are 5-methylcytosine, p=0 and q=4 and all occurrences of Y are abasic sites.

More preferred oligonucleotides of this second aspect have been described in the experimental part and comprise or consist of SEQ ID NO:16, 17, 19 20.

A preferred oligonucleotide comprises SEQ ID NO:16 and has a length of 21, 22, 23, 24, 25, 26, 27, 28, 29, 30 nucleotides.

Another preferred oligonucleotide comprises SEQ ID NO:17 (21 nucleotides and 4 abasic sites) and has a length of 21, 22, 23, 24, 25, 26, 27, 28, 29, 30 nucleotides and the 4 abasic sites.

Another preferred oligonucleotide comprises SEQ ID NO:19 or 20 and has a length of 15, 16, 17, 18, 19, 20, 21, 22, 23, 24, 25, 26, 27, 28, 29, 30 nucleotides.

Oligonucleotide Comprising Abasic Sites

In a third aspect, the present invention relates to a oligonucleotide, which comprises one or more abasic sites, as defined further below, at one or both termini. Preferably 2 to 20, more preferably 3 to 10, most preferably 4 abasic sites are present at a single terminus of the oligonucleotide. One or more abasic sites may be present and both free termini of the oligonucleotide (5′ and 3′), or at only one. The oligonucleotide according to this aspect of the invention preferably comprises (NAG)ₘ, wherein N and m are as defined above, and may further optionally comprise any of the modification as discussed herein, such as one or more base modification, sugar modification and/or backbone modification, such as 5-methylcytosine, 2,6-diaminopurine, 2′-O-methyl, phosphorothioate, and combinations thereof.

The oligonucleotide according to this aspect of the invention, comprising one or more abasic sites at one or both termini has an improved parameter over the oligonucleotides without such abasic sites as explained later herein.

Oligonucleotide Part or Oligonucleotide

In the next section, the oligonucleotide according to the invention is further defined. This disclosure is applicable to the oligonucleotide part of the conjugate comprising or consisting of LGAQSNF/(NAG)ₘ (i.e. first aspect) to the oligonucleotide comprising or consisting of (NAG)ₘ (i.e. second aspect) and to the oligonucleotide comprising or consisting of (NAG)ₘ which comprises one or more abasic sites at one or both termini (i.e. third aspect) unless explicitly stated otherwise. Thus, throughout the description, “oligonucleotide according to the invention” can be replaced by either “oligonucleotide part of the conjugate comprising or consisting of LGAQSNF/(NAG)ₘ” or by “oligonucleotide comprising or consisting of (NAG).” or by “oligonucleotide comprising or consisting of (NAG)ₘ which comprises one or more abasic sites”.

The oligonucleotide according to the invention may have 9 to 90 or 9 to 60 or 9 to 45 or 9 to 42 or 9 to 39 or 9 to 36 nucleotides or 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 or 90 nucleotides. It is therefore clear that the invention also encompasses any specific oligonucleotide that can be designed by starting and/or finishing at any position in the given NAG (in which N is C or 5-methylcytosine) without prejudice that one or the other resulting sequences could be more efficient.

In an embodiment, the oligonucleotide according to the invention or the conjugate comprising or consisting of LGAQSNF/(NAG)ₘ may further comprise an additional oligonucleotide part which is complementary to a sequence present in a cell from an individual to be treated. This additional oligonucleotide part may for example be a sequence complementary to a sequence flanking the CUG repeat present in the transcript of a DM1/DMPK (SEQ ID NO: 10), SCA8 (SEQ ID NO: 11) or JPH3 (SEQ ID NO: 12) gene. Or, this additional oligonucleotide part may for example be a sequence complementary to a sequence not directly flanking the repeat sequence CUG in the transcript of a DM1/DMPK, SCA8 or JPH3 gene. Or, this additional oligonucleotide part may for example be a sequence complementary to a sequence not directly flanking the repeat sequence CUG present in the transcript of a DM1/DMPK, SCA8 or JPH3 gene, and contain a functional motif. Or, this additional oligonucleotide part may for example be a sequence complementary to a sequence not directly flanking the repeat sequence CUG present in the transcript of a DM1/DMPK, SCA8 or JPH3 gene, but in proximity because of the secondary or tertiary structure. Preferably, the sequence (NAG)ₘ in which N is C or 5-methylcytosine is at least 50% of the length of the oligonucleotide according to the invention, more preferably at least 60%, even more preferably at least 70%, even more preferably at least 80%, even more preferably at least 90% or more. In this respect, one or more abasic sites present at one or both of the termini of the oligonucleotide according to the invention are not part of the sequence. In a more preferred embodiment, the oligonucleotide according to the invention consists of (NAG)ₘ in which N is C or 5-methylcytosine. Even more preferably, the oligonucleotide according to the invention consists of (NAG)ₘ in which N is 5-methylcytosine. Even more preferably, the oligonucleotide according to the invention consists of (NAG)₇ in which N is 5-methylcytosine.

The oligonucleotide according to the invention may be single stranded or double stranded. Double stranded means that the oligonucleotide is a heterodimer made of two complementary strands, such as in a siRNA. In a preferred embodiment, the oligonucleotide according to the invention is single stranded. The skilled person will understand that it is however possible that a single stranded oligonucleotide may form an internal double stranded structure. However, this oligonucleotide is still named as a single stranded oligonucleotide in the context of this invention. A single stranded oligonucleotide has several advantages compared to a double stranded siRNA oligonucleotide: (i) its synthesis is expected to be easier than two complementary siRNA strands; (ii) there is a wider range of chemical modifications possible to optimise more effective uptake in cells, a better (physiological) stability and to decrease potential generic adverse effects; (iii) siRNAs have a higher potential for non-specific effects (including off-target genes) and exaggerated pharmacology (e.g. less control possible of effectiveness and selectivity by treatment schedule or dose) and (iv) siRNAs are less likely to act in the nucleus and cannot be directed against introns.

Different types of nucleic acid monomers may be used to generate the oligonucleotide according to the invention. The oligonucleotide according to the invention may have at least one backbone modification, and/or at least one sugar modification and/or at least one base modification compared to an RNA-based oligonucleotide.

A base modification includes a modified version of the natural purine and pyrimidine bases (e.g. adenine, uracil, guanine, cytosine, and thymine), such as hypoxanthine, orotic acid, agmatidine, lysidine, 2-thiopyrimidine (e.g. 2-thiouracil, 2-thiothymine), 2,6-diaminopurine, G-clamp and its dervatives, 5-substituted pyrimidine (e.g. 5-halouracil, 5-methyluracil, 5-methylcytosine, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 8-aza-7-deazaguanine, 8-aza-7-deazaadenine, 8-aza-7-deaza-2,6-diaminoadenine, Super G, Super A, and N4-ethylcytosine, or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen). An oligonucleotide according to the invention may comprise 1, 2, 3, 4, 5, 6, 7, 8, 9, 10 or more base modifications. Examples of derivatives of Super A, Super G and Super T can be found in U.S. Pat. No. 6,683,173 (Epoch Biosciences), which is incorporated here entirely by reference. It is also encompassed by the invention to introduce more than one distinct base modification in said oligonucleotide part.

An oligonucleotide according to the invention (i.e. first, second, third aspect) preferably comprises a modified base and/or an basic site all as identified herein since it is expected to provide a compound or an oligonucleotide of the invention with an improved RNA binding kinetics and/or thermodynamic properties, provide a compound or an oligonucleotide of the invention with a decreased or acceptable level of toxicity and/or immunogenicity, and/or enhance pharmacodynamics, pharmacokinetics, activity, allele selectivity, cellular uptake and/or potential endosomal release of the oligonucleotide or compound of the invention.

In a more preferred embodiment, one or more 2-thiouracil, 2-thiothymine, 5-methylcytosine, 5-methyluracil, thymine, 2,6-diaminopurine bases is present in said oligonucleotide according to the invention. As indicated above, the oligonucleotide according to the invention which is not conjugated to a peptide part, i.e. the oligonucleotide as represented by H—(X)ₚ—(NAG)ₘ-(Y)_{q}—H, comprises at least one base modification selected from 5-methylcytosine (5-methyl-C) and 2,6-diaminopurine. In a preferred embodiment, the oligonucleotide according to this aspect of the invention, which is not conjugated with a peptide part, does not comprise a hypoxanthine base modification. A sugar modification includes a modified version of the ribosyl moiety, such as 2′-O-alkyl or 2′-O-(substituted)alkyl (e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-allyl, 2′-O-(2-amino)propyl, 2′-O-(2-(dimethylamino)propyl), 2′-O-(2-amino)ethyl and 2′-O-(2-(dimethylamino)ethyl)); 2′-deoxy (DNA), 2′-O-alkoxycarbonyl (e.g. 2′-O-[2-(methoxycarbonyl)ethyl] (MOCE), 2′-O-[2-(N-methylcarbamoyl)ethyl] (MCE) and 2′-O-[2-(N,N-dimethylcarbamoyl)ethyl] (DCME)), 2′-halo (e.g. 2′-F, FANA (2′-F arabinosyl nucleic acid)); carbasugar and azasugar modifications; and 3′-O-alkyl (e.g. 3′-O-methyl, 3′-O-butyryl, 3′-O-propargyl, and derivatives thereof). Another possible modification includes “bridged” or “bicylic” nucleic acid (BNA), e.g. locked nucleic acid (LNA), xylo-LNA, α-L-LNA, β-D-LNA, cEt (2′-O,4′-C constrained ethyl) LNA, cMOEt (2′-O,4′-C constrained methoxyethyl) LNA, ethylene-bridged nucleic acid (ENA); unlocked nucleic acid (UNA); cyclohexenyl nucleic acid (CeNA), altriol nucleic acid (ANA), hexitol nucleic acid (HNA), fluorinated HNA (F—HNA), pyranosyl-RNA (p-RNA), 3′-deoxypyranosyl-DNA (p-DNA); tricyclo-DNA (tcDNA); morpholino (PMO), cationic morpholino (PMOPlus), PMO-X; and their derivatives. The oligonucleotide according to the invention may comprise 1, 2, 3, 4, 5, 6, 7, 8, 9, 10 or more sugar modifications. It is also encompassed by the invention to introduce more than one distinct sugar modification in said oligonucleotide.

In a preferred embodiment, the oligonucleotide according to the invention comprises at least one sugar modification selected from 2′-O-methyl, 2′-O-(2-methoxy)ethyl, morpholino, a bridged nucleotide or BNA, or the oligonucleotide comprises both bridged nucleotides and 2′-deoxy modified nucleotides (BNA/DNA mixmers or gapmers), or both 2′-O-(2-methoxy)ethyl nucleotides and DNA nucleotides (2′-O-(2-methoxy)ethyl/DNA mixmers or gapmers). More preferably, the oligonucleotide according to the invention is modified over its full length with a sugar modification selected from 2′-O-methyl, 2′-O-(2-methoxy)ethyl, morpholino, bridged nucleic acid (BNA), 2′-O-(2-methoxy)ethyl/DNA mixmer, 2′-O-(2-methoxy)ethyl/DNA gapmer, BNA/DNA gapmer or BNA/DNA mixmer. In an even more preferred embodiment, the oligonucleotide according to the invention comprises at least one 2′-O-methyl modification. In a more preferred embodiment, an oligonucleotide according to the invention is fully 2′-O-methyl modified.

In a preferred embodiment, the oligonucleotide according to the invention comprises 1-10 or more monomers that lack the nucleobase. Such monomer may also be called an abasic site or an abasic monomer. Such monomer may be present or linked or attached or conjugated to a free terminus of the oligonucleotide of the invention.

When the oligonucleotide according to the invention is represented by H—(X)ₚ—(NAG)ₘ-(Y)_{q}—H, abasic sites may be present within the (X)ₚ portion of the oligonucleotide and/or the (Y)_{q} portion of the oligonucleotide. When the oligonucleotide according to the invention is present within the compound represented by LGAQSNF/(NAGₘ, abasic sites may be present at a free terminus of the oligonucleotide part. These abasic sites may be present at the terminal regions of the oligonucleotide, i.e. at the 5′-terminus and/or at the 3′-terminus. Also, the oligonucleotide part of the conjugate may comprise abasic sites. These abasic site may be attached to a free terminus of said oligonucleotide part of the conjugate. Because of the conjugation with the peptide part, only one of the termini may be free. Thus, the 3′-terminus is free when the peptide is conjugated via the 5′-terminus, or the 5′-terminus is free when the peptide is conjugated via the 3′-terminus. On the other hand, conjugation with the peptide part may also occur via a nucleotide or other moiety present within the oligonucleotide part, which leaves both the 5′- and the 3′-terminus free and thus available for attachment of one or more abasic sites.

Apart from the abasic sites present at the free termini of the oligonucleotide according to the invention, abasic sites may also be present within the oligonucleotide sequence. In this respect, abasic sites are considered base modifications.

In a more preferred embodiment, the oligonucleotide according to the invention comprises 1-10 or more abasic sites or monomers of 1-deoxyribose, 1,2-dideoxyribose, and/or 1-deoxy-2-O-methylribose. Such monomer(s) may be present at a free terminus of the oligonucleotide of the invention. The number of monomers may be 1, 2, 3, 4, 5, 6, 7, 8, 9, 10, 11, 12, 13, 14, 15, 16, 17, 18, 19, 20 or even more. Attachment of a number of these abasic monomers in an oligonucleotide of the invention shows increased activity with respect to a control oligonucleotide that does not comprise such monomers. These monomers may be attached to the 3′ or the 5′ terminal nucleotide, or to both. The abasic monomers may be attached in regular 5′→3′ sequence or reversed (3′→5′) fashion and may be linked to each other and to the remainder of the oligonucleotide according to the invention through phosphate, phosphorothioate or phosphodiamidate bonds. In a preferred embodiment, 2-8 abasic sites or monomers are attached to the 3′ or the 5′ end of the oligonucleotide of the invention. In a more preferred embodiment, 4 abasic sites or monomers are attached at the 3′ terminus of the (NAG)ₘ oligonucleotide according to the invention. Even more preferably, 4 abasic sites or monomers are attached at the 3′ terminus of the (NAG)₇ oligonucleotide of the invention. In a most preferred embodiment, an oligonucleotide of the invention comprises 4 monomers of 1-deoxyribose, 1,2-dideoxyribose, and/or 1-deoxy-2-O-methylribose that are present at the 3′ terminus of said oligonucleotide of the invention, preferably wherein said oligonucleotide of the invention is (NAG)₇.

The RNA binding kinetics and/or thermodynamic properties are at least in part determined by the melting temperature of an oligonucleotide of the invention (Tm; calculated with the oligonucleotide properties calculator (http://www.unc.edu/˜cail/biotool/oligo/index.html) for single stranded RNA using the basic Tm and the nearest neighbour model, of the oligonucleotide according to the invention bound to its target RNA (using RNA structure version 4.5).

Immunogenicity may be assessed in an animal model by assessing the presence of CD4⁺ and/or CD8⁺ cells and/or inflammatory mononucleocyte infiltration in muscle biopsy of said animal. Immunogenicity and/or toxicity may also be assessed in blood of an animal or of a human being treated with a compound or an oligonucleotide of the invention or an oligonucleotide part of said compound by detecting the presence of an antibody recognizing said compound or oligonucleotide of the invention or an oligonucleotide part of said compound using a standard immunoassay known to the skilled person.

Toxicity may be assessed in blood of an animal or a human being treated with a compound or an oligonucleotide of the invention or an oligonucleotide part of said compound by detecting the presence of a cytokine and/or by detecting complement activation. In this context, a cytokine may be IL-6, TNF-α, IFN-α and/or IP-10. The presence of each of these cytokines may be assessed using ELISA, preferably sandwich ELISA. The ELISA kit from R&D Systems may be used to assess the presence of human IL-6, TNF-α, IL-10, or from Verikine for IFN-α, or from Invitrogen for monkey IL-6 and TNF-α. Complement activation may be assessed by ELISA by assessing the presence of Bb and C3a. A suitable ELISA to this end is from Quidel (Calif., San Diego).

An increase in immunogenicity preferably corresponds to a detectable increase of at least one of these cell types by comparison to the amount of each cell type in a corresponding muscle biopsy of an animal before treatment or treated with a compound or an oligonucleotide of the invention or an oligonucleotide part of said compound having no modified bases. Alternatively, an increase in immunogenicity may be assessed by detecting the presence or an increasing amount of an antibody recognizing said compound or oligonucleotide of the invention or an oligonucleotide part of said compound using a standard immunoassay.

A decrease in immunogenicity preferably corresponds to a detectable decrease of at least one of these cell types by comparison to the amount of corresponding cell type in a corresponding muscle biopsy of an animal before treatment or treated with a corresponding compound or oligonucleotide of the invention or an oligonucleotide part of said compound having no modified base. Alternatively a decrease in immunogenicity may be assessed by the absence of or a decreasing amount of said compound or oligonucleotide of the invention or an oligonucleotide part of said compound and/or neutralizing antibodies using a standard immunoassay.

An increase in toxicity preferably corresponds to a detectable increase of a cytokine as identified above and/or to a detectable increase of complement activation by comparison to the situation of an animal before treatment or treated with a compound or oligonucleotide of the invention or an oligonucleotide part of said compound having no modified bases.

A decrease in toxicity preferably corresponds to a detectable decrease of a cytokine as identified above and/or to a detectable decrease of the complement activation of an animal before treatment or treated with a corresponding compound or oligonucleotide of the invention or an oligonucleotide part of said compound having no modified base.

A backbone modification includes a modified version of the phosphodiester present in RNA. In this respect, the term “backbone” is to be interpreted as the internucleoside linkage. Examples of such backbone modifications are phosphorothioate (PS), chirally pure phosphorothioate, phosphorodithioate (PS2), phosphonoacetate (PACE), phosphonoacetamide (PACA), thiophosphonoacetate, thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, and their derivatives. Other possible modifications include phosphoramidite, phosphoramidate, N3′→P5′ phosphoramidate, phosphordiamidate, phosphorothiodiamidate, sulfamate, dimethylenesulfoxide, sulfonate, thioacetamido nucleic acid (TANA), and their derivatives. An oligonucleotide according to the invention may comprise 1, 2, 3, 4, 5, 6, 7, 8, 9, 10 or more backbone modifications. It is also encompassed by the invention to introduce more than one distinct backbone modification in said oligonucleotide of the invention.

In a preferred embodiment, an oligonucleotide according to the invention comprises at least one phosphorothioate modification. In a more preferred embodiment, an oligonucleotide of the invention is fully phosphorothioate modified.

Other chemical modifications of an oligonucleotide according to the invention include peptide nucleic acid (PNA), boron-cluster modified PNA, pyrrolidine-based oxy-peptide nucleic acid (POPNA), glycol- or glycerol-based nucleic acid (GNA), threose-based nucleic acid (TNA), acyclic threoninol-based nucleic acid (aTNA), morpholino-based oligonucleotide (PMO, PMO-X), cationic morpholino-based oligomers (PMOPlus), oligonucleotides with integrated bases and backbones (ONIBs), pyrrolidine-amide oligonucleotides (POMs), and their derivatives. In a preferred embodiment, the oligonucleotide according to the invention is modified with morpholino-based nucleotides (PMO) or peptide nucleotides (PNA) over its entire length.

With the advent of nucleic acid mimicking technology it has become possible to generate molecules that have a similar, preferably the same hybridisation characteristics in kind not necessarily in amount as nucleic acid itself. Such functional equivalents are of course also suitable for use in the invention.

The skilled person will understand that not each sugar, base, and/or backbone may be modified the same way. Several distinct sugar, base and/or backbone modifications may be combined into one single oligonucleotide according to the invention.

A person skilled in the art will also recognize that there are many synthetic derivatives of oligonucleotides. Therefore, “oligonucleotide” includes, but is not limited to phosphodiesters, phosphotriesters, phosphorothioates, phosphodithioates, phosphorothiodiamidate and H-phosphonate derivatives. It encompasses also both naturally occurring and synthetic oligonucleotide derivatives.

Preferably, said oligonucleotide according to the invention comprises RNA, as RNA/RNA duplexes are very stable. It is preferred that an RNA oligonucleotide comprises a modification providing the RNA with an additional property, for instance resistance to endonucleases, exonucleases, and RNaseH, additional hybridisation strength, increased stability (for instance in a bodily fluid), increased or decreased flexibility, reduced toxicity, increased intracellular transport, tissue-specificity, etc. Preferred modifications have been identified above.

Preferably, said oligonucleotide according to the invention comprises or consists of 2′-O-methyl RNA monomers connected through a phosphorothioate backbone. Such an oligonucleotide consisting of 2′-O-methyl RNA monomers and a phosphorothioate backbone can also be referred to as “2′-O-methyl phosphorothioate RNA”. Also, when only a portion of the oligonucleotide according to the invention consists of 2′-O-methyl RNA monomers and a phosphorothioate backbone, this portion can be referred to as “2′-β-methyl phosphorothioate RNA”. The oligonucleotide according to the invention then comprises 2′-O-methyl RNA monomers connected through a phosphorothioate backbone or 2′-O-methyl phosphorothioate RNA. One embodiment thus provides an oligonucleotide according to the invention which comprises RNA further containing a modification, preferably a 2′-O-methyl modified ribose (RNA), more preferably a 2′-O-methyl phosphorothioate RNA.

Hybrids between one or more of the equivalents among each other and/or together with nucleic acid are of course also suitable.

Oligonucleotide according to the invention containing at least in part naturally occurring DNA nucleotides are useful for inducing degradation of DNA-RNA hybrid molecules in the cell by RNase H activity (EC.3.1.26.4).

Naturally occurring RNA ribonucleotides or RNA-like synthetic ribonucleotides comprising oligonucleotides according to the invention are encompassed herein to form double stranded RNA-RNA hybrids that act as enzyme-dependent antisense through the RNA interference or silencing (RNAi/siRNA) pathways, involving target RNA recognition through sense-antisense strand pairing followed by target RNA degradation by the RNA-induced silencing complex (RISC).

Alternatively or in addition, the oligonucleotide according to the invention can interfere with the processing or expression of precursor RNA or messenger RNA (steric blocking, RNase-H independent processes) in particular but not limited to RNA splicing and exon skipping, by binding to a target sequence of RNA transcript and getting in the way of processes such as translation or blocking of splice donor or splice acceptor sites. Moreover, the oligonucleotide according to the invention may inhibit the binding of proteins, nuclear factors and others by steric hindrance and/or interfere with the authentic spatial folding of the target RNA and/or bind itself to proteins that originally bind to the target RNA and/or have other effects on the target RNA, thereby contributing to the destabilization of the target RNA, preferably mRNA, and/or to the decrease in amount of diseased or toxic transcript thereby leading to a decrease of nuclear accumulation of ribonuclear foci in diseases like DM1 as identified later herein.

As herein defined, an oligonucleotide according to the invention may comprise nucleotides with (RNaseH resistent) chemical substitutions at at least one of its 5′ or 3′ ends, to provide intracellular stability, and comprises less than 9, more preferably less than 6 consecutive (RNaseH-sensitive) deoxyribose nucleotides in the rest of its sequence. The rest of the sequence is preferably the center of the sequence. Such oligonucleotide is called a gapmer.

Gapmers have been extensively described in WO 2007/089611. Gapmers are designed to enable the recruitment and/or activation of RNaseH. Without wishing to be bound by theory, it is believed that RNaseH is recruited and/or activated via binding to the central region of the gapmer made of deoxyriboses. The oligonucleotide according to the invention which is preferably substantially independent of RNaseH is designed in order to have a central region which is substantially not able to recruit and/or activate RNaseH. In a preferred embodiment, the rest of the sequence of the oligonucleotide of the invention, more preferably its central part comprises less than 9, 8, 7, 6, 5, 4, 3, 2, 1, or no deoxyribose. Accordingly this oligonucleotide according to the invention is preferably partly till fully substituted as earlier defined herein. Partly substituted preferably means that the oligonucleotide according to the invention comprises at least 50% of its nucleotides that have been substituted, at least 55%, 60%, 65%, 70%, 75%, 80%, 85%, 90%, 95%, or 100% (i.e. fully) substituted.

As indicated above, the oligonucleotide according to the invention as represented by H—(X)ₚ—(NAG)ₘ-(Y)_{q}—H preferably does not comprise inosine as nucleotide or hypoxanthine as nucleobase.

On the other hand, when the oligonucleotide according to the invention is part of a conjugate with a peptide part, said oligonucleotide part preferably contains or comprises an inosine and/or a nucleotide containing a base able to form a Wobble base pair. More preferably said oligonucleotide part comprises an inosine. In the current invention, a compound comprising an oligonucleotide part comprising at least one inosine is attractive. In an especially preferred embodiment, in (NAG)ₘ all or almost all occurrences of A are replaced by inosine (I). When all occurrences of A are replaced by I, the oligonucleotide according to the invention comprises m occurrences of I. “Almost all occurrence of A replaced by I” is to be understood as that m—1, 2 or 3 occurrences of A are replaced by I. Such compound can be used to treat at least two diseases, myotonic dystrophy 1 which is caused by a (CUG)ₙ expanded repeat, and e.g. Huntington's disease, which is caused by a (CAG)ₙ expanded repeat. Specifically targeting these expansion repeats would otherwise require two compounds, each compound comprising one distinct oligonucleotide part. An oligonucleotide part comprising an inosine and/or a nucleotide containing a base able to form a wobble base pair may be defined as an oligonucleotide wherein at least one nucleotide has been substituted with an inosine and/or a nucleotide containing a base able to form a Wobble base pair. The skilled person knows how to test whether a nucleotide contains a base able to form a Wobble base pair. Since for example inosine can form a base pair with uracil, adenine, and/or cytosine, it means that at least one nucleotide able to form a base pair with uracil, adenine and/or cytosine has been substituted with inosine. However, in order to safeguard specificity, the inosine containing oligonucleotide preferably comprises the substitution of at least one nucleotide able to form a base pair with uracil or adenine or cytosine. More preferably, all nucleotides able to form a base pair with uracil or adenine or cytosine are substituted with inosine. An oligonucleotide part complementary to a repeat sequence (CUG)ₙ will preferably comprise or consist of (NIG)ₙ in which N is C or 5-methylcytosine. It is also to be encompassed by the present invention that since at least one nucleotide has been substituted by inosine and/or a nucleotide containing a base able to form a Wobble base pair in an oligonucleotide part as defined herein, that an oligonucleotide part complementary to a repeat sequence such as (CUG)ₙ may comprise or consist of (NIG)ₙ in which N is C or 5-methylcytosine. If one takes (NIG)ₙ in which N is C or 5-methylcytosine as example, having n as 3 as example, the invention encompasses any possible oligonucleotide part based on a given formula such as (NIG)₃ comprising 1 or 2 or 3 inosine(s) at the indicated position: (NAG)(NIG)(NAG), (NIG)(NAG)(NAG), (NIG)(NAG)(NIG), (NIG)(NIG)(NAG), (NIG)(NIG)(NIG) (in which N is C or 5-methylcytosine). It is to be understood that the (NAG)ₘ part of the oligonucleotide part of the compound of the invention may comprise of consists of (NIG)ₙ. In this respect, n is an integer which is equal to or smaller than m. In a preferred embodiment, n is equal to m, and thus in the compound of the invention, (NAG)ₘ part of the oligonucleotide part consists of (NIG)ₘ. In this embodiment, at least one of adenine nucleobases contains a base modification, in particular a hypoxanthine nucleobase. Preferably, the (NAG)ₘ part of the oligonucleotide part of the compound of the invention comprises 1, 2, 3, 4, 5, . . . , m hypoxanthine nucleobases.

Thus, in a preferred embodiment the oligonucleotide according to the invention comprises:
(a) at least one base modification selected from 2-thiouracil, 2-thiothymine, 5-methylcytosine, 5-methyluracil, thymine, 2,6-diaminopurine; and/or
(b) at least one sugar modification selected from 2′-O-methyl, 2′-O-(2-methoxy)ethyl, morpholino, a bridged nucleotide or BNA, or the oligonucleotide comprises both bridged nucleotides and 2′-deoxy modified nucleotides (BNA/DNA mixmers or gapmers), or both 2′-O-(2-methoxy)ethyl nucleotides and DNA nucleotides (2′-O-(2-methoxy)ethyl/DNA mixmers or gapmers); and/or
(c) at least one backbone modification selected from phosphorothioate and phosphordiamidate.

In another preferred embodiment, the oligonucleotide according to the invention is modified over its entire length with one or more of the same modification, selected from (a) one of the base modifications; and/or (b) one of the sugar modifications; and/or (c) one of the backbone modifications.

In a preferred embodiment, the oligonucleotide or the oligonucleotide part of the compound according to the invention comprises at least one modification selected from the group consisting of 2′-O-methyl phosphorothioate, morpholino phosphorodiamidate, locked nucleic acid and peptide nucleic acid. In a more preferred embodiment, the oligonucleotide or oligonucleotide part of the compound according to the invention comprises one or more 2′-O-methyl phosphorothioate monomers. In a more preferred embodiment, the oligonucleotide or oligonucleotide part of the compound according to the invention consists of 2′-O-methyl phosphorothioate monomers. In other words, it is preferred that the oligonucleotide part of the compound according to the invention is a 2′-O-methyl phosphorothioate oligonucleotide. In a preferred embodiment, the oligonucleotide or oligonucleotide part of the compound according to the invention comprises at least one base selected from 2,6-diaminopurine, 2-thiouracil, 2-thiothymine, 5-methyluracil, thymine, 8-aza-7-deazaguanosine, and/or hypoxanthine.

Linking Part of the Conjugate Represented by LGAQSNF/(NAG)ₘ

In order to prepare the compound according to the first aspect of the present invention, which can be represented by LGAQSNF/(NAG)ₘ, coupling of the oligonucleotide part to the peptide or peptidomimetic part according to this aspect of the present invention occurs via known methods to couple compounds to amino acids or peptides. A common method is to link a moiety to a free amino group or free hydroxyl group or free carboxylic acid group or free thiol group in a peptide or peptidomimetic. Common conjugation methods include thiol/maleimide coupling, amide or ester or thioether bond formation, or heterogeneous disulfide formation. The skilled person is well aware of standard chemistry that can be used to bring about the required coupling. The oligonucleotide part may be coupled directly to the peptide part or may be coupled via a spacer or linker molecule. Such a spacer or linker may be divalent, thus linking one peptide or peptidomimetic part with one oligonucleotide part, or multivalent. Multivalent spacers or linkers may be used to link more than one peptide or peptidomimetic part with one oligonucleotide part. Divalent and multivalent linkers or spacers are known to the skilled person. It is not necessary that the oligonucleotide part is covalently linked to the peptide or peptidomimetic part according to this aspect of the invention. It may also be associated or conjugated via electrostatic interactions. Such a non-covalent linkage is also subject of the present invention, and is to be understood as encompassed in the terms “link” and “linkage”. In one embodiment the present invention also relates to a compound comprising a peptide or peptidomimetic part according to this aspect of the invention and a linking part, for linking the peptide part to the oligonucleotide part. The linking part may not be a peptide or may be a peptide. The linking part for example may be a (poly)cationic group that complexes with a biologically active poly- or oligonucleotide. Such a (poly)cationic group may be a linear or branched version of spermine or polyethyleneimine, poly-ornithine, poly-lysine, poly-arginine and the like. The linking part may also be neutral as for example a linking part comprising or consisting of polyethylene glycol.

The peptide or peptidomimetic part of a compound according the first aspect of the invention can be linked, coupled or conjugated to the oligonucleotide part via the C-terminus, via the N-terminus or via a side chain of an amino acid, and could be linked to the 5′-terminal nucleotide, the 3′-terminal nucleotide or a non-terminal nucleotide through the base, backbone or sugar moiety of that particular nucleotide of the oligonucleotide part.

Any possible known way of coupling or linking an oligonucleotide part to a peptide part may be used in this aspect of the present invention to obtain a compound according to this aspect of the invention. A peptide part may be coupled or linked to an oligonucleotide part through a linkage including, but not limited to, linkers comprising a thioether, amide, amine, oxime, disulfide, thiazolidine, urea, thiourea, ester, thioester, carbamate, thiocarbamate, carbonate, thiocarbonate, hydrazone, sulphate, sulphamidate, phosphate, phosphorothioate, or glyoxylic-oxime moiety, or a linkage obtained via Diels-Alder cycloaddition, Staudinger ligation, native ligation or Huisgen 1,3-dipolar cycloaddition or the copper catalyzed variant thereof. In a preferred embodiment, the linkage comprises a thioether moiety. In one embodiment, the invention provides a compound comprising a peptide part comprising LGAQSNF and an oligonucleotide part comprising (NAG)ₘ in which N is 5-methylcytosine, wherein said compound is represented by formula A.

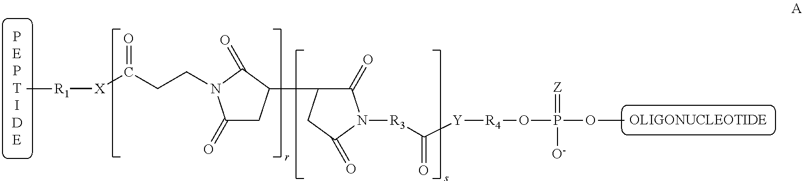

In which

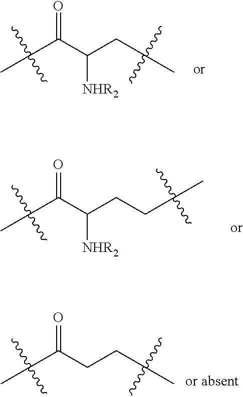

### R₁ is

R₂ is acetyl or H;
R₃ is substituted or unsubstituted (C₁-C₁₀)alkyl, (C₁-C₁₀)cycloalkyl, aryl or (C₁-C₁₀)aralkyl;
R₄ is (C₁-C₁₅)alkyl, ethylene glycol, diethylene glycol, triethylene glycol, tetraethylene glycol, polyethylene glycol or derivative;

### X is S, C═O or NH;

### Y is S or NH;

### Z is S or O;

r and s are 0 or 1, provided that r+s=0 or 1,
wherein R₁ is connected via an amide or ester bond with an amine or alcohol at the N-terminus, C-terminus or a side chain of an amino acid of the peptide part;
wherein R₄ is connected to the 5′ or 3′ of the oligonucleotide part.

Preferably, X═S or NH when r=1.

In a preferred embodiment, this aspect of the invention provides a compound represented by any of the formulae I-VII

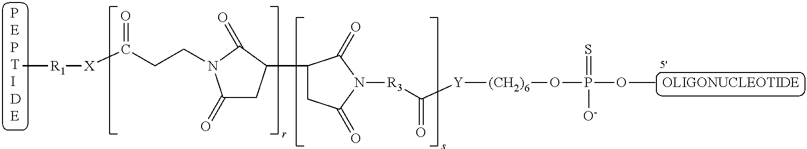

| COMPOUND | R₁ | R₂ | R₃ | X | Y | r | s |
|---|---|---|---|---|---|---|---|
| I | absent | — | — | NH | S | 1 | 0 |
| II | absent | — | — | C═O | NH | 0 | 0 |
| III | 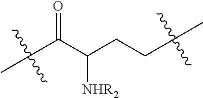 | acetyl | — | C═O | NH | 0 | 0 |
| IV | 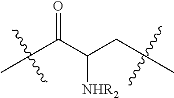 | H | ethyl | S | NH | 0 | 1 |
| V | 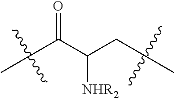 | H | cyclohexyl | S | NH | 0 | 1 |
| VI | 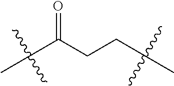 [SMILES: *CCC(*)=O] | — | cyclohexyl | S | NH | 0 | 1 |
| VII | 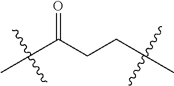 [SMILES: *CCC(*)=O] | — | cyclohexyl | S | NH | 0 | 1 |
| VIII | 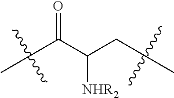 | acetyl | ethyl | S | NH | 0 | 1 |

In the compound according to formula I, X is the N-terminal amino group of the peptide part; in the compound according to formula II, X is the C-terminal carboxyl group of the peptide part; in any of the compounds according to the formulae III-VIII, R₁ is connected to the N-terminus of the peptide part via an amide bond. In compounds V, VI and VII, “cyclohexyl” is understood to be “cyclohexane-1,4-diyl” or “1,4-cyclohexanediyl”. The conjugation represented in formula I is well-known to the skilled person and is preferably synthesized as explained in the examples. Likewise, other methods of conjugation are known in the art or will be known in the art. The peptide part could be linked to the oligonucleotide part from the N-terminus, C-terminus or a side chain of an amino acid; and could be linked from the 5′-terminal nucleotide. The skilled person understands that the peptide part may also be linked to the 3′-terminal nucleotide or a non-terminal monomer through the base, backbone or sugar moiety of that particular monomer.

Equally preferred compounds according to this aspect of the invention are identical to compounds I-VIII, except that the oligonucleotide is attached via its 3′-terminus to the linking part.

In case an abasic site or monomer is present or attached to a terminus of the oligonucleotide part of the compound of the invention, the peptide part is attached not to the same terminus. Thus, in case a peptide part is coupled to the 5′ terminus of the oligonucleotide part, then—if incorporated—the abasic site or monomer is attached to the 3′ terminus of the oligonucleotide part.

Peptide Part of the Conjugate Represented by LGAQSNF/(NAG)ₘ

As already indicated above, the peptide part of the compound according to this aspect of the invention comprises or consists of LGAQSNF. A peptide part in the context of this aspect of the invention comprises at least 7 amino acids. A compound according to this aspect of the invention may comprise more than one peptide part as identified herein: a compound according to this aspect of the invention may comprise 1, 2, 3, 4, 5, 6, 7, 8 peptide parts linked to an oligonucleotide part, all as identified herein. The peptide can be fully constructed of naturally occurring L-amino acids, or can contain one or more modifications to backbone and/or side chain(s) with respect to L-amino acids. These modifications can be introduced by incorporation of amino acid mimetics that show similarity to the natural amino acid. The group of peptides described above comprising one or more mimetics of amino acids is referred to as peptidomimetics. In the context of this aspect of the invention, mimetics of amino acids include, but are not limited to, β²- and β³-amino acids, β^{2,2}-β^{2,3}, and β^{3,3}-disubstituted amino acids, α,α-disubstituted amino acids, statine derivatives of amino acids, D-amino acids, α-hydroxyacids, α-aminonitriles, N-alkylamino acids and the like. Additionally, amino acids in the peptide part of this aspect of the invention may be glycosylated with one or more carbohydrate moieties and/or derivatives, or may be phosphorylated.

In addition, the C-terminus of the peptide might be carboxylic acid or carboxamide, or other resulting from incorporation of one of the above mentioned amino acid mimetics. Furthermore, the peptide part described above may contain one or more replacements of native peptide bonds with groups including, but not limited to, sulfonamide, retroamide, aminooxy-containing bond, ester, alkylketone, α,α-difluoroketone, α-fluoroketone, peptoid bond (N-alkylated glycyl amide bond). Furthermore, the peptide part mentioned above may contain substitutions in the amino acid side chain (referring to the side chain of the corresponding natural amino acid), for instance 4-fluorophenylalanine, 4-hydroxylysine, 3-aminoproline, 2-nitrotyrosine, N-alkylhistidine or β-branched amino acids or β-branched amino acid mimetics with chirality at the β-side chain carbon atom opposed to the natural chirality (e.g. allo-threonine, allo-isoleucine and derivatives). In one other embodiment, above mentioned peptide may contain close structural analogues of amino acid or amino acids mimetics, for instance ornithine instead of lysine, homophenylalanine or phenylglycine instead of phenylalanine, β-alanine instead of glycine, pyroglutamic acid instead of glutamic acid, norleucine instead of leucine or the sulfur-oxidized versions of methionine and/or cysteine. The linear and cyclized forms of the peptide part mentioned above are covered by this patent, as well as their retro, inverso and/or retroinverso analogues. To those skilled in the art many more close variations may be known, but the fact that these are not mentioned here does not limit the scope of the present invention. In one embodiment, a peptide part or peptidomimetic part according to this aspect of the present invention is at most 30 amino acids in length, or at least 25 amino acids or 20 amino acids or 19, 18, 17, 16, 15, 14, 13, 12, 11, 10, 9, 8 or 7 amino acids in length. A preferred peptide part comprises or consists of LGAQSNF and at least 0, 1, 2, 3 or more amino acids at the N-terminus and/or at the C-terminus: for example XXXLGAQSNFXXX, wherein X may be any amino acid.

Application

A compound or oligonucleotide of the invention is particularly useful for treating, delaying and/or preventing and/or treating and/or curing and/or ameliorating a human genetic disorder as myotonic dystrophy type 1, spino-cerebellar ataxia 8 and/or Huntington's disease-like 2 caused by repeat expansions in the transcripts of DM1/DMPK, SCA8 or JPH3 genes respectively. Preferably, these genes are from human origin. A preferred genomic DNA sequence of a human DMPK, respectively SCA8, JPH3 gene is represented by SEQ ID NO: 10, 11, 12. A corresponding preferred coding cDNA sequence of a human DMPK, respectively SCA8, JPH3 gene is represented by SEQ ID NO: 13, 14, 15.

In a preferred embodiment, in the context of the invention, a compound or oligonucleotide as designed herein is able to delay and/or cure and/or treat and/or prevent and/or ameliorate a human genetic disorder as myotonic dystrophy type 1, spino-cerebellar ataxia 8 and/or Huntington's disease-like 2 caused by CUG repeat expansions in the transcript of the DM1/DMPK, SCA8 or JPH3 genes when this compound or oligonucleotide is able to reduce or decrease the number of CUG repeats in the transcript of a diseased allele of a DM1/DMPK, SCA8 or JPH3 gene in a cell of a patient, in a tissue of a patient and/or in a patient.

Although in the majority of patients, a “pure” CUG repeat is present in a transcribed gene sequence in the genome of said patient. However, it is also encompassed by the invention, that in some patients, said repeat is not qualified as “pure” or is qualified as a “variant” when for example said repeat is interspersed with at least 1, 2, or 3 nucleotide(s) that do not fit the nucleotide(s) of said repeat (Braida C., et al,).

An oligonucleotide according to the invention may not be 100% reverse complementary to a targeted CUG repeat. Usually an oligonucleotide of the invention may be at least 90%, 95%, 97%, 99% or 100% reverse complementary to a CUG repeat.

In the case of DM1, a CUG repeat is present in exon 15 of the DMPK transcript. A CUG repeat may be herein defined as a consecutive repetition of at least 30, 35, 38, 39, 40, 45, 50, 55, 60, 70, 100, 200, 500 of the repetitive unit CUG or more comprising a trinucleotide repetitive unit CUG, in a transcribed gene sequence of the DMPK gene in the genome of a subject, including a human subject.

In the case of spino-cerebellar ataxia 8, the repeat expansion is located in the 3′UTR of the SCA8 gene. The SCA8 locus is bidirectionally transcribed and produces RNAs with either (CUG)ₙ or (CAG)ₙ expansions. (CAG)ₙ expansion transcripts produce a nearly pure polyglutamine (polyQ) protein. A CUG or a CAG repeat may be herein defined as a consecutive repetition of at least 65, 70, 75, 80, 100, 200, 500 of the repetitive unit CUG or more comprising a CUG trinucleotide repetitive unit respectively of the repetitive unit CAG comprising a CAG trinucleotide repetitive unit, in a transcribed gene sequence of the SCA8 gene in the genome of a subject, including a human subject.

Huntington's disease-like 2 is caused by a (CUG)ₙ expansion in the transcript of the JPH3 gene. Depending on the alternative splicing of the JPH3 transcript, the CUG repeat could lie in an intron, in the 3′ UTR or in a coding region encoding a polyleucine or polyalanine tract. A CUG repeat may be herein defined as a consecutive repetition of at least 35, 40, 41, 45, 50, 50, 55, 60 or more, of the repetitive unit CUG comprising a trinucleotide repetitive unit CUG, in a transcribed gene sequence of the JPH3 gene in the genome of a subject, including a human subject.

Throughout the invention, the term CUG repeat may be replaced by (CUG)ₙ wherein n is an integer that may be 10, 20, 30 or not higher than 30 when the repeat is present in exon 15 of the DMPK transcript of a healthy individual, 20, 30, 40, 50, 60, 65 or not higher than 65 when the repeat is present in the SCA8 gene of a healthy individual or 10, 20, 30, 35 or not higher than 35 when the repeat is present in the JPH3 gene of a healthy individual. In the case of DM1, spino-cerebellar ataxia 8 or Huntington's patients, n may have other value as indicated above.

It preferably means that the compound or oligonucleotide of the invention reduces the detectable amount of disease-associated or disease-causing or mutant transcript containing an extending or unstable number of CUG repeats in a cell of said patient, in a tissue of said patient and/or in a patient. Alternatively or in combination with previous sentence, said compound may reduce the translation of said mutant transcript. The reduction or decrease of the number of CUG repeats or of the quantity of said mutant transcript may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the number of CUG repeats or of the quantity of said mutant transcript before the treatment. The reduction may be assessed by Northern Blotting or Q-RT-PCR, preferably as carried out in the experimental part. A compound or oligonucleotide of the invention may first be tested in the cellular system as used in the experimental comprising a 500 CUG repeat. Alternatively or in combination with previous preferred embodiment, in the context of the invention, a compound or an oligonucleotide of the invention as designed herein is able to delay and/or cure and/or treat and/or prevent and/or ameliorate a human genetic disorder as myotonic dystrophy type 1, spino-cerebellar ataxia 8 and/or Huntington's disease-like 2 caused by a CUG repeat expansion in the transcript of the DM1/DMPK, SCA8 or JPH3 genes when this compound or oligonucleotide is able to alleviate one or more symptom(s) and/or characteristic(s) and/or to improve a parameter linked with or associated with myotonic dystrophy type 1, spino-cerebellar ataxia 8 and/or Huntington's disease-like 2 in an individual. A compound or oligonucleotide as defined herein is able to improve one parameter or reduce a symptom or characteristic if after at least one week, one month, six month, one year or more of treatment using a dose of the compound or oligonucleotide of the invention as identified herein said parameter is said to have been improved or said symptom or characteristic is said to have been reduced.

Improvement in this context may mean that said parameter had been significantly changed towards a value of said parameter for a healthy person and/or towards a value of said parameter that corresponds to the value of said parameter in the same individual at the onset of the treatment.

Reduction or alleviation in this context may mean that said symptom or characteristic had been significantly changed towards the absence of said symptom or characteristic which is characteristic for a healthy person and/or towards a change of said symptom or characteristic that corresponds to the state of the same individual at the onset of the treatment.

In this context, a preferred symptom for myotonic dystrophy type 1 is myotonia, muscle strength or stumbles and falls. Each of these symptoms may be assessed by the physician using known and described methods.

Myotonia could be assessed using an EMG (ElectroMyoGram): an EMG is a quantitative test of handgrip strength, myotonia, and/or fatigue in myotonic dystrophy, (Tones C. et al,) as known to the skilled person. If there is a detectable reduction in myotonia as assessed by EMG towards an EMG pattern of a healthy person, preferably after at least one week, one month, six month, one year or more of treatment using a dose of the compound of the invention as identified herein, we preferably conclude that said myotonia has been reduced or alleviated.

Other preferred symptoms of myotonic dystrophy type 1 are muscle strength (Hebert et al.) or a reduction in stumbles and falls (Wiles, et al,). Here also, If there is a detectable improvement of muscle strength or detectable reduction of stumbles and falls towards muscle strength or stumbles and falls of a healthy person, preferably after at least one week, one month, six month, one year or more of treatment using a dose of the compound or an oligonucleotide of the invention as identified herein, we preferably conclude that said muscle strength has been improved or that said stumbles and falls has been reduced or alleviated.

In this context, a preferred symptom for spino-cerebrellar ataxia 8 includes ataxia, proprioceptive and coordination defects including gait impairment and a general lack of motor control, including upper motor neuron dysfunction, dysphagia, peripheral sensory disturbances. Each of these symptoms may be assessed by the physician using known and described methods: ataxia may be assessed by the physician using known and described methods: such as static posturography or dynamic posturography. Static posturography essentially measures various aspects of balance and sway. While little is documented on the use of techniques for diagnosing the presence of a symptom associated with SCA8, we assumed that techniques used for diagnosing the same symptom in other closely related indications as SCA6 could be used for diagnosing SCA8 (Nakamura et al, Januario et al,). For example the ICARS (International Cooperative Ataxia Rating Score) may be used for diagnosing SCA8 (assessed in Nakamura et al, or Trouillas P. et al,). As another example, the OASI (Overall Stability Index) may be used for diagnosing SCA8 (assessed in Januario et al,).

For more refined motor function skills, common hand function tests such as the Jebson timed test the Perdue Pegboard test or 9 peg hole test may be considered, although again, not specific to, or validated in, this indication. If there is a detectable reduction in at least one of these symptoms of spino-cerebrellar ataxia 8 or a detectable change of the ICARS and/or OASI assessed as described above towards the value of said symptom or of said ICARS or OASI of a healthy person, preferably after at least one week, one month, six month, one year or more of treatment using a dose of the compound or oligonucleotide of the invention as identified herein, we preferably conclude that said symptom or said ICARS or OASI has been reduced or alleviated or changed using a compound of the invention.

In this context, a preferred symptom for Huntington's disease-like 2 includes chorea and/or dystonia chorea and/or dystonia. Each of these symptoms may be assessed by the physician using known and described methods. They may be diagnosed by genetic testing (Walker, et al) and by clinical assessment with the use of scales such as the Unified Huntington's Disease Rating Scale Movement Disorders Vol. I I, No. 2, 1996, pp. 136-142, and Mahant et al,). If there is a detectable reduction in at least one of these symptoms of Huntington's disease-like 2 assessed as described above towards the value of said symptom of a healthy person, preferably after at least one week, one month, six month, one year or more of treatment using a dose of the compound or oligonucleotide of the invention as identified herein, we preferably conclude that said symptom has been reduced or alleviated using a compound or oligonucleotide of the invention.

A parameter for myotonic dystrophy type 1 may be the splicing pattern of certain transcripts (for example C1C-1, SERCA, IR, Tnnt, Tau). Myotonic dystrophy is characterized by an embryonic splicing pattern for a wide variety of transcripts (Aberrant alternative splicing and extracellular matrix gene expression in mouse models of myotonic dystrophy; Hongquing D. et al). A splicing pattern of these genes could be visualised using PCR or by using genomic screens. When the embryonic splicing pattern of at least one of the genes identified above had been found altered towards wild type splicing pattern of the corresponding gene after at least one month, six month or more of treatment with a dose of a compound or an oligonucleotide of the invention as identified herein, one could say that a compound or an oligonucleotide of the invention is able to improve a parameter linked with or associated with myotonic dystrophy type 1 in an individual.

Another parameter for myotonic dystrophy type 1 may be insulin resistance (measured by blood glucose and HbAlc levels), the normal ranges of which are 3.6-5.8 mmol/L and 3-8 mmol/L respectively. Reduction of these values towards or within the normal range would indicate a positive benefit. When at least one of these values had been found altered towards wild type values after at least one month, six month or more of treatment with a dose of a compound or oligonucleotide of the invention as identified herein, one could say that a compound or oligonucleotide of the invention is able to improve a parameter linked with or associated with myotonic dystrophy type 1 in an individual.

Another parameter for myotonic dystrophy type 1 is the number of RNA-MBNL (muscle blind protein) foci or nuclear inclusions in the nucleus which could be visualized using fluorescence in situ hybridization (FISH). DM1 patients have 5 to 20 RNA-MBNL foci in their nucleus (Taneja K L et al,). A nuclear inclusion or foci may be defined as an aggregate or an abnormal structure present in the nucleus of a cell of a DM1 patient and which is not present in the nucleus of a cell of a healthy person. When the number of foci or nuclear inclusions in the nucleus is found to have changed (analyzed with FISH) and preferably to be decreased by comparison to the number of nuclear foci or nuclear inclusions at the onset of the treatment, one could say that a compound or an oligonucleotide of the invention is able to improve a parameter linked with or associated with myotonic dystrophy in an individual. The decrease of the number of foci or nuclear inclusions may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the number of foci or nuclear inclusions at the onset of the treatment. Preferably, the muscle blind protein MBNL is detached from these foci or nuclear inclusions (as may be analyzed with immunofluorescence microscopy) and more preferably free available in the cell. The decrease of the number of RNA-MBNL may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the number of RNA-MBNL at the onset of the treatment. A free available MBNL in the cell may be detected using immunofluorescence microscopy: a more diffuse staining of MBNL will be seen and less to no co-localization with nuclear (CUG)ₙ foci or nuclear inclusions anymore.

A parameter for spino-cerebellar ataxia 8 includes a decrease or a lowering of the amount of polyglutamine protein (preferably assessed by Western blotting) and/or a decrease or a lowering of the number of nuclear polyglutamine inclusions (preferably assessed by immunofluorescence microscopy). Beside the (CAG)ₙ transcripts that form polyglutamine protein inclusions, (CUG)ₙ transcripts form nuclear inclusions or foci could bevisualized using FISH. The presence of a polyglutamine protein and nuclear inclusion is preferably assessed in neurons. A nuclear inclusion or foci may be defined as an aggregate or an abnormal structure present in the nucleus of a cell of a spino-cerebellar ataxia 8 patient and which is not present in the nucleus of a cell of a healthy person. When the number of foci or nuclear inclusions in the nucleus is found to have changed (analyzed with FISH) and preferably to be decreased by comparison to the number of nuclear foci or nuclear inclusions at the onset of the treatment, one could say that a compound or an oligonucleotide of the invention is able to improve a parameter linked with or associated with spino-cerebellar ataxia 8 in an individual. The decrease of the number of foci or nuclear inclusions may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the number of foci or nuclear inclusions at the onset of the treatment. A decrease of the amount of quantity of a polyglutamine protein may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity of said protein detected at the onset of the treatment. Another parameter would be the decrease in (CUG)ₙ transcript or of the quantity of said mutant transcript. This may be of at least. 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity of said transcript detected at the onset of the treatment A parameter for Huntington's disease-like 2 includes the decrease of or lowering the pathogenic polyleucine or polyalanine tracts (Western blotting and immunofluorescence microscopy). A decrease of the amount or of quantity of the polyleucine or polyalanine tract may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity of said tract assessed at the onset of the treatment. Another parameter would be the decrease in (CUG)ₙ transcript or of the quantity of said mutant transcript. This may be of at least 1%, 5%, 10%, 15%, 20%, 25%, 30%, 35%, 40%, 45%, 50%, 55%, 60%, 65%, 70%, 75%, 80%, 85%, 90%, 95%, 100% by comparison to the quantity of said transcript detected at the onset of the treatment. Another parameter for Huntington's disease-like 2 includes the number of RNA-MBNL (muscleblind protein) foci in the nucleus as for myotonic dystrophy.

A compound or an oligonucleotide according to the invention is suitable for direct administration to a cell, tissue and/or organ in vivo of an individual affected by or at risk of developing myotonic dystrophy type 1, spino-cerebellar ataxia 8 and/or Huntington's disease-like 2, and may be administered directly in vivo, ex vivo or in vitro. An individual or a subject or a patient is preferably a mammal, more preferably a human being. A tissue or an organ in this context may be blood.

In a preferred embodiment, a concentration of a compound or an oligonucleotide is ranged from 0.01 nM to 1 μM is used. More preferably, the concentration used is from 0.05 to 400 nM, or from 0.1 to 400 nM, or from 0.02 to 400 nM, or from 0.05 to 400 nM, even more preferably from 1 to 200 nM. Preferred concentrations are from 0.01 nM to 1 μM. More preferably, the concentration used is from 0.3 to 400 nM, even more preferably from 1 to 200 nM.

Dose ranges of a compound or an oligonucleotide according to the invention are preferably designed on the basis of rising dose studies in clinical trials (in vivo use) for which rigorous protocol requirements exist. A compound or an oligonucleotide as defined herein may be used at a dose which is ranged from 0.01 to 500 mg/kg, or from 0.01 to 250 mg/kg or 0.01 to 200 mg/kg or 0.05 to 100 mg/kg or 0.1 to 50 mg/kg or 0.1 to 20 mg/kg, preferably from 0.5 to 10 mg/kg.

The ranges of concentration or dose of compound or oligonucleotide as given above are preferred concentrations or doses for in vitro or ex vivo uses. The skilled person will understand that depending on the identity of the compound or oligonucleotide used, the target cell to be treated, the gene target and its expression levels, the medium used and the transfection and incubation conditions, the concentration or dose of compound or oligonucleotide used may further vary and may need to be optimised any further.

More preferably, a compound or oligonucleotide used in the invention to prevent, treat or delay myotonic dystrophy type 1, spino-cerebellar ataxia 8 and/or Huntington's disease-like 2 is synthetically produced and administered directly to a cell, a tissue, an organ and/or a patient or an individual or a subject in a formulated form in a pharmaceutically acceptable composition. Administration of a compound or oligonucleotide of the invention may be local, topical, systemic and/or parenteral. The delivery of said pharmaceutical composition to the subject is preferably carried out by one or more parenteral injections, e.g. intravenous and/or subcutaneous and/or intramuscular and/or intrathecal and/or intranasal and/or intraventricular and/or intraperitoneal, ocular, urogenital, enteral, intravitreal, intracerebral, intrathecal, epidural and/or oral administrations, preferably injections, at one or at multiple sites in the human body. An intrathecal or intraventricular administration (in the cerebrospinal fluid) is preferably realized by introducing a diffusion pump into the body of a subject. Several diffusion pumps are known to the skilled person. Pharmaceutical compositions that are to be used to target a compound or an oligonucleotide as defined herein may comprise various excipients such as diluents, fillers, preservatives, solubilisers and the like, which may for instance be found in Remington et al. The compound as described in the invention may possess at least one ionizable group. An ionizable group may be a base or acid, and may be charged or neutral. An ionizable group may be present as ion pair with an appropriate counterion that carries opposite charge(s). Examples of cationic counterions are sodium, potassium, cesium, Tris, lithium, calcium, magnesium, trialkylammonium, triethylammonium, and tetraalkylammonium. Examples of anionic counterions are chloride, bromide, iodide, lactate, mesylate, acetate, trifluoroacetate, dichloroacetate, and citrate. Examples of counterions have been described (e.g. Kumar et al., which is incorporated here in its entirety by reference). A compound or an oligonucleotide of the invention may be prepared as a salt form thereof. Preferably, it is prepared in the form of its sodium salt. A compound or oligonucleotide of the present invention may optionally be further formulated in a composition which may be a pharmaceutically acceptable solution or composition containing pharmaceutically accepted diluents and carriers, and to which pharmaceutically accepted additives may be added to bring the formulation to desired pH and/or osmolality, for example solution or dilution in sterile water or phosphate buffer and brought to desired pH with acid or base, and to desired osmolality with organic or inorganic salts. For example, HCl may be used to bring a solution to the desired pH, whereas NaCl may be used to bring a solution to desired osmolality.

A pharmaceutical composition may comprise an excipient in enhancing the stability, solubility, absorption, bioavailability, activity, pharmacokinetics, pharmacodynamics and cellular uptake of said compound or oligonucleotide, in particular an excipient capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, gold nanoparticles, magnetic nanoparticles, silica nanoparticles, lipid nanoparticles, sugar particles, protein nanoparticles and peptide nanoparticles.

In an embodiment a compound or an oligonucleotide of the invention may be used together with another compound already known to be used for treating, delaying and/or preventing and/or treating and/or curing and/or ameliorating a human genetic disorder as myotonic dystrophy type 1, spino-cerebellar ataxia 8 and/or Huntington's disease-like 2 caused by repeat expansions in the transcripts of DM1/DMPK, SCA8 or JPH3 genes respectively. Such other compound may be a steroid. This combined use may be a sequential use: each component is administered in a distinct composition. Alternatively each compound may be used together in a single composition.

In a method of the invention, we may use an excipient that will further aid in enhancing the stability, solubility, absorption, bioavailability, activity, pharmacokinetics, pharmacodynamics and delivery of said compound or oligonucleotide to a cell and into a cell, in particular excipients capable of forming complexes, vesicles, nanoparticles, microparticles, nanotubes, nanogels, hydrogels, poloxamers or pluronics, polymersomes, colloids, microbubbles, vesicles, micelles, lipoplexes and/or liposomes, that deliver compound, substances and/or oligonucleotide(s) complexed or trapped in the vesicles or liposomes through a cell membrane. Examples of nanoparticles include gold nanoparticles, magnetic nanoparticles, silica nanoparticles, lipid nanoparticles, sugar particles, protein nanoparticles and peptide nanoparticles. Another group of delivery systems are polymeric nanoparticles. Many of these substances are known in the art. Suitable substances comprise polymers (e.g. polyethylenimine (PEI), ExGen 500, polypropyleneimine (PPI), poly(2-hydroxypropylenimine (pHP)), dextran derivatives (e.g. polycations such like diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver said compound across cell membranes into cells), butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine), lipids {e.g. 1,2-dioleoyl-3-dimethylammonium propane (DODAP), dioleoyldimethylammonium chloride (DODAC), phosphatidylcholine derivatives [e.g 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyeline, 2-{3-[bis-(3-amino-propyl)-amino]-propylamino}-N-ditetracedyl carbamoyl methylacetamide (RPR209120), phosphoglycerol derivatives [e.g. 1,2-dipalmitoyl-sn-glycero-3-phosphoglycerol, sodium salt (DPPG-Na), phosphaticid acid derivatives [1,2-d]stearoyl-sn-glycero-3-phosphaticid acid, sodium salt (DSPA), phosphatidylethanolamine derivatives [e.g. dioleoyl-L-R-phosphatidylethanolamine (DOPE),1,2-distearoyl-sn-glycero-3-phosphoethanolamine (DSPE),2-diphytanoyl-sn-glycero-3-phosphoethanolamine (DPhyPE)], N-[1-(2,3-dioleoyloxy)propyl]-N,N,N-trimethylammonium (DOTAP), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-Arginyl-2,3-L-diaminopropionic acid-N-palmityl-N-olelyl-amide trihydrochloride (AtuFECT01), N,N-dimethyl-3-aminopropane derivatives [e.g. 1,2-distearoyloxy-N,N-dimethyl-3-aminopropane (DSDMA), 1,2-dioleyloxy-N,N-dimethyl-3-aminopropane (DoDMA), 1,2-dilinoleyloxy-N,N-3-dimethylaminopropane (DLinDMA), 2,2-dilinoleyl-4-dimethylaminomethyl [1,3]-dioxolane (DLin-K-DMA), phosphatidylserine derivatives [1,2-dioleyl-sn-glycero-3-phospho-L-serine, sodium salt (DOPS)], cholesterol}, synthetic amphiphils (SAINT-18), lipofectin, proteins (e.g. albumin, gelatins, atellocollagen), peptides (e.g., PepFects, NickFects, polyarginine, polylysine, CADY, MPG), combinations thereof and/or viral capsid proteins that are capable of self assembly into particles that can deliver said compound or oligonucleotide to a cell. Lipofectin represents an example of liposomal transfection agents. It consists of two lipid components, a cationic lipid N-[1-(2,3-dioleoyloxy)propyl]-N,N,N-trimethylammonium chloride (DOTMA) (cp. DOTAP which is the methylsulfate salt) and a neutral lipid dioleoylphosphatidylethanolamine (DOPE). The neutral component mediates the intracellular release.

In addition to these nanoparticle materials, the cationic peptide protamine offers an alternative approach to formulate said compound or oligonucleotide as colloids. This colloidal nanoparticle system can form so called proticles, which can be prepared by a simple self-assembly process to package and mediate intracellular release of a compound as defined herein. The skilled person may select and adapt any of the above or other commercially available or not commercially available alternative excipients and delivery systems to package and deliver a compound or oligonucleotide for use in the current invention to deliver such compound or oligonucleotide for treating, preventing and/or delaying of myotonic dystrophy type 1, spino-cerebellar ataxia 8 and/or Huntington's disease-like 2 in humans.

In addition, another ligand could be covalently or non-covalently linked to a compound or oligonucleotide specifically designed to facilitate its uptake in to the cell, cytoplasm and/or its nucleus. Such ligand could comprise (i) a compound (including but not limited to a peptide(-like) structure) recognising cell, tissue or organ specific elements facilitating cellular uptake and/or (ii) a chemical compound able to facilitate the uptake in to a cell and/or the intracellular release of said compound or oligonucleotide from vesicles, e.g. endosomes or lysosomes. Such targeting ligand would also encompass molecules facilitating the uptake of said compound or oligonucleotide into the brain through the blood brain barrier. Within the context of the invention, a peptide part of the compound of the invention may already be seen as a ligand.

Therefore, in a preferred embodiment, a compound or an oligonucleotide as defined herein is part of a medicament or is considered as being a medicament and is provided with at least an excipient and/or a targeting ligand for delivery and/or a delivery device of said compound or oligonucleotide to a cell and/or enhancing its intracellular delivery. Accordingly, the invention also encompasses a pharmaceutically acceptable composition comprising said compound or oligonucleotide and further comprising at least one excipient and/or a targeting ligand for delivery and/or a delivery device of said compound to a cell and/or enhancing its intracellular delivery.

However, due to the presence of a peptide part comprising LGAQSNF in a conjugate of the invention, the use of such excipient and/or a targeting ligand for delivery and/or a delivery device of said compound to a cell and/or enhancing its intracellular delivery is preferably not needed.

The invention also pertains to a method for alleviating one or more symptom(s) and/or characteristic(s) and/or for improving a parameter of myotonic dystrophy type 1, spino-cerebellar ataxia 8 and/or Huntington's disease-like 2 in an individual, the method comprising administering to said individual a compound or an oligonucleotide or a pharmaceutical composition as defined herein.

In this document and in its claims, the verb “to comprise” and its conjugations is used in its non-limiting sense to mean that items following the word are included, but combinations and/or items not specifically mentioned are not excluded. In the context of the invention, contains preferably means comprises.

In addition the verb “to consist” may be replaced by “to consist essentially of” meaning that a compound or a composition as defined herein may comprise additional component(s) than the ones specifically identified, said additional component(s) not altering the unique characteristic of the invention.

The word “about” or “approximately” when used in association with a numerical value (about 10) preferably means that the value may be the given value of 10 more or less 1% of the value.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

The present invention is further described by the following examples which should not be construed as limiting the scope of the invention.

### FIGURE LEGENDS

FIG. 1. Reagents and conditions: a. maleimide propionic acid, HCTU, DIPEA; b. TFA/H₂O/TIS 95/2.5/2.5, ambient temperature, 4 h; c. Thiol modifier C6 S—S phosphoramidite, ETT; d. PADS, 3-picoline; e. concentrated ammonium hydroxide (NH₄OH), 0.1M DTT, 55° C., 16 h; f. Sodium phosphate buffer 50 mM, 1 mM EDTA, ambient temperature 16 h. The peptide (SEQ ID NO:2) is attached via its N terminus (amino acid L) to the oligonucleotide. For this reason, in this figure the peptide is depicted as FNSQAGL from C to N terminal. The resulting LGAQSNF-PS58 is a conjugate according to the first aspect of the invention. Herein, “PS58” designates the oligonucleotide part of said conjugate (SEQ ID NO: 1), which is (NAG)₇ wherein N is C, and which is a 2′-O-methyl phosphorothioate RNA. This conjugate can also be represented by LGAQSNF/(CAG)₇. Throughout the figures and the figure legends, “LGAQSNF-PS58” is used to indicate the conjugate as prepared by the process according to FIG. 1, and “PS58” is used to indicate an oligonucleotide consisting of (NAG)₇ wherein N is C, and which is modified with 2′-O-methyl phosphorothioate over its entire length, which is optionally conjugated to a peptide or peptidomimetic part.

FIG. 2. LGAQSNF/(CAG)₇ mediated silencing of expanded hDMPK transcripts in DM500 cells. Northern blot analysis indicated that a peptide conjugated version of PS58 (LGAQSNF-PS58 or LGAQSNF/(CAG)₇) was still functional (lanes with PEI, number of experiments (n)=3, P<0.01) and was able to enter the cell nucleus causing silencing of expanded hDMPK transcripts without (w/o) the use of a transfection reagent (n=3, P<0.001). Gapdh was used as loading control.

FIG. 3. Injection scheme intramuscular injection with LGAQSNF/PS58 (CAG)₇. Eight DM500 mice were injected in the left GPS complex with LGAQSNF-PS58 (LGAQSNF/(CAG)₇). In the right GPS complex four of these mice were injected with PS58 ((CAG)₇) and four mice were injected with LGAQSNF-23 (“23” represents an unrelated control AON (SEQ ID NO:3)). Mice were sacrificed and muscles were isolated one (n=4 for LGAQSNF-PS58 and n=2 for PS58 and LGAQSNF-23) or three days (n=4 for LGAQSNF-PS58 and n=2 for PS58 and LGAQSNF-23) after the final injection.

FIG. 4. LGAQSNF/(CAG)₇ shows proof-of-concept in DM500 mice in vivo after intramuscular injection. In DM500 mice, injection of LGAQSNF-PS58 (LGAQSNF/(CAG)₇) in the GPS complex followed by quantitative RT-PCR analysis of RNA content confirmed silencing of hDMPK (CUG)₅₀₀ mRNA in the gastrocnemius, plantaris and soleus after LGAQSNF-PS58 treatment compared to (A) PS58 ((CAG)₇; SEQ ID NO:1)) or (B) LGAQSNF-23 (“23” represents an unrelated control AON (SEQ ID NO:3)) treatment. (C) A significant reduction in all tissue was found when LGAQSNF-PS58 treatment was compared to both controls. (A-C) Data is grouped per tissue regardless of isolation day, two-tailed paired t-test, *P<0.05, **P<0.01, ***P<0.001.

FIG. 5. Silencing capacities of modified AONs targeted towards the (CUG)ₙ repeat. Quantitative RT-PCR analysis indicated that PS387, (NAG)₇ wherein N=5-methylcytosine (SEQ ID NO: 16) (n=3, P<0.05), and PS613 (NAG)₇XXXX wherein N═C and X=1,2-dideoxyribose abasic site (SEQ ID NO: 17) (n=3, P<0.01) significantly reduce mutant (CUG)ₙ transcripts in the in vitro DM500 cell model after transfection compared to mock treated cells (n=81). PS58 ((CAG)₇) (SEQ ID NO:1) was included as a positive control (n=26, P<0.001). Gapdh and β-actin were used as loading control.

FIG. 6. Synthesis of LGAQSNF/(NAG)₇: a conjugate wherein the peptide (SEQ ID NO: 2) is linked to a fully 2′-O-methyl phosphorothioate modified RNA oligonucleotide (NAG)₇, wherein N═C (SEQ ID NO:1) (11) or 5-methylcytosine (SEQ ID NO:16) (12), through a bifunctional crosslinker. Reagents and conditions: a. TFA/H₂O/TIS 95/2.5/2.5, ambient temperature, 4 h; b. MMT-amino modifier C6 phosphoramidite, ethylthiotetrazole; c. PADS, 3-picoline; d. conc. ammonium hydroxide, 55° C., 16 h.; e. AcOH:H₂O (80:20 v:v); f. DMSO-phosphate buffer, ambient temperature, 16 h.; g. sodium phosphate buffer (50 mM), 1 mM EDTA, ambient temperature, 16 h.

FIG. 7. Comparative analysis of the activity of AONs designed to target the expanded (CUG). repeat in hDMPK (CUG)₅₀₀ transcripts in differentiated DM500 cells in vitro, including (NAG)₇ wherein N═C in PS58 (SEQ ID NO: 1) or N=5-methylcytosine in PS387 (SEQ ID NO: 16), and (NZG)₅ wherein N═C and Z=A in PS147 (SEQ ID NO: 18), or N=5-methylcytosine and Z=A in PS389 (SEQ ID NO:19), or N═C and Z=2,6-diaminopurine in PS388(SEQ ID NO:20), all at a fixed transfection concentration of 200 nM. Their activity, i.e. silencing of hDMPK transcripts, was quantified by quantitative RT-PCR using primers in exon 15. hDMPK transcript levels after AON treatment were compared to the relative corresponding levels in the mock samples. For all AONs n=3 except for mock (n=81), PS58 (n=26). “n” represents the number of experiments carried out. Statistical analysis was performed on AONs with similar length. The presence of 5-methylcytosines had a significant positive effect on the activity of both the (CAG)₅ and (CAG)₇ AONs. The presence of 2,6-diaminopurines allowed the shorter (CAG)₅ AON to have a similar activity as the longer (CAG)₇ AON. Differences between groups were considered significant when P<0.05. *P<0.05, **P<0.01, ***P<0.001.

FIG. 8. Analysis of DM500 mice treated subcutaneously with LGAQSNF/(CAG)₇ ((CAG)₇ is represented by PS58; SEQ ID NO: 1) for four consecutive days at a 100 mg/kg dose per day, one day after last injection. A control group was included in which the mice were treated with LGAQSNF/control AON (the control AON is a scrambled PS58 sequence as represented by SEQ ID NO: 21). Levels of hDMPK (CUG)₅₀₀ RNA were quantified by Q-RT-PCR analysis with primers 5′ of the (CUG). repeat in exon 15. Treatment with LGAQSNF-PS58 (LGAQSNF/(CAG)₇, as prepared with the process according to FIG. 1, resulted both in gastrocnemius (A) as in heart (B) in a reduction of expanded hDMPK levels compared to mice treated with LGAQSNF/control AON. Differences between groups were considered significant when P<0.05. *P<0.05.

FIG. 9. Analysis of HSA^{LR} mice treated subcutaneously with LGAQSNF/(CAG)₇, as prepared with the process according to FIG. 1 ((CAG)₇ is represented by PS58; SEQ ID NO: 1) for five consecutive days at a 250 mg/kg dose per day, 4 weeks after last injection. (A) EMG (electromyogram) measurements were performed on a weekly base by an examiner blinded for mouse identity. A significant reduction in myotonia was observed in gastrocnemius muscle in treated mice as compared to saline-injected mice. (B) Northern blot analysis revealed reduced levels of toxic (CUG)₂₅₀ mRNA in gastrocnemius muscle in treated mice compared to saline-injected mice. (C) RT-PCR analysis demonstrated a reduction in embryonic splice mode (i.e. shift towards a more adult splicing pattern) of the chloride channel (Clcn1), serca (Serca1) and titin (Ttn) transcripts in gastrocnemius muscle of treated mice compared to saline-injected mice.

FIG. 10. Analysis of HSA^{LR} mice treated subcutaneously with LGAQSNF/(CAG)₇, as prepared with the process according to FIG. 1 ((CAG)₇ is represented by PS58; SEQ ID NO: 1) by 11 injections of 250 mg/kg in a 4 week period, 4 days after the last injection. Northern blot analysis demonstrated that long-term treatment resulted in a significant reduction of toxic (CUG)₂₅₀ levels, both in gastrocnemius muscle (10a, left graph) as in tibialis anterior (10a, right graph graph) compared to saline-injected mice. RT-PCR analysis demonstrated a reduction in embryonic splice mode (i.e. shift towards a more adult splicing pattern) of the chloride channel (Clcn1), serca (Serca1) and titin (Ttn) transcripts in both gastrocnemius (10b, left graph) and tibialis anterior (10b, right graph graph) muscles of treated mice compared to control. Differences between groups were considered significant when P<0.05. *P<0.05, **P<0.01, ***P<0.001.

### EXAMPLES

### Example 1

### Synthesis PP08-P558 Conjugate

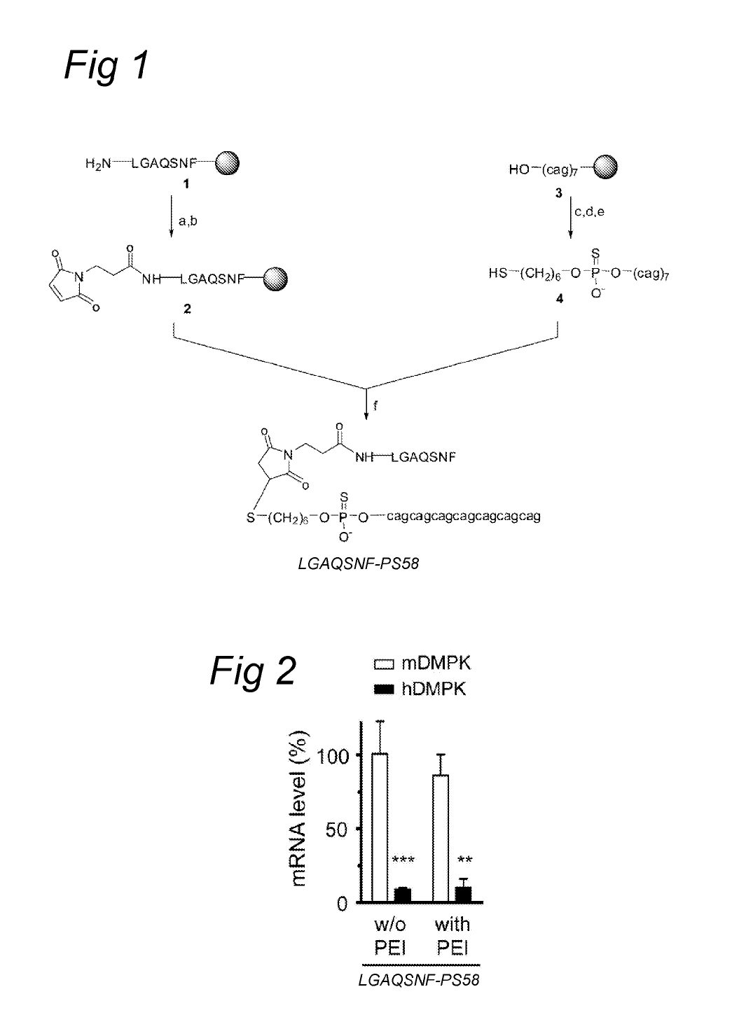

LGAQSNF-PS58 (LGAQSNF/(CAG)₇, wherein (CAG)₇ is represented by SEQ ID NO:1) was synthesized following a procedure adapted from the one of Ede N. J. et al. The preparation of LGAQSNF-PS58 conjugate is depicted in FIG. 1.

Peptide 1 (SEQ ID NO:2) was synthesized by standard Fmoc solid phase synthesis. On line coupling of maleimide propionic acid, followed by deprotection and cleavage of the resin with TFA:H₂O:TIS 95:2.5:2.5 and subsequent purification by reversed phase HPLC afforded peptide 2 in 38% yield.

Thiol modifier C6 S—S phosphoramidite was coupled to oligonucleotide 3 via phosphorothioate bond on solid support. Treatment of the crude resin with 40% aqueous ammonia and 0.1 M DTT led to the concomitant cleavage of the solid support, deprotection of the nucleobases and reduction of the disulfide bond. Thiol containing oligonucleotide 4 was isolated in 52% yield after reversed phase HPLC purification. Immediately before conjugate, compound 4 was applied to a PD-10 column with phosphate buffer 50 mM, at pH=7. Eluted fractions containing the free thiol oligonucleotide 4 were directly conjugated to peptide 2 (5 eq) via thiol-maleimide coupling at room temperature for 16 hours. The crude was purified by reversed phase HPLC and LGAQSNF-PS58 was isolated in 40% yield.

Experimental Part

Chemicals

For peptide synthesis, Fmoc amino acids were purchased from Orpegen, 2-(6-Chloro-1H-benzotriazole-1-yl)-1,1,3,3-tetramethylaminium hexafluorophosphate (HCTU) from PTI, Rink amide MBHA Resin from Novabiochem and 3-maleimidopropionic acid from Bachem. For oligonucleotide synthesis, 2′-O-Me RNA phosphoramidites were obtained from ThermoFisher and Thiol-Modifier C6 S—S phosphoramidite was obtained from ChemGenes. Custom Primer Support and PD-10 columns were from GE-Healthcare. 1,4-dithiothreitol (DTT) and phenylacetyl disulfide (PADS) were purchased from Sigma-Aldrich and American International Chemical, respectively.

Peptide Synthesis

The synthesis of peptide 1 was carried out on a Tribute (Protein Technologies Inc.) peptide synthesizer by standard Fmoc chemistry. Rink amide MBHA resin (0.625 mmol/g, 160 mg, 100 μmol) was used for the synthesis. Fmoc deprotection was accomplished using 20% piperidine in N-methylpyrrolidone (NMP) and at every coupling 5 eq. Fmoc amino acid, 5 eq. HCTU and 10 eq. N,N-diisopropylethylamine (DIPEA) were added to the resin and coupling proceeded for 1 hour. After peptide sequence 1 was completed, 3-maleimidopropionic acid (5 eq) was coupled on line under the same conditions as described before. Deprotection and cleavage from the resin was achieved using trifluoroacetic acid (TFA):H₂O:triisopropylsilane (TIS) 95:2.5:2.5 for 4 hours at room temperature. The mixture was precipitated in cold diethylether and centrifuged. The precipitate was purified by reversed phase (RP)HPLC on a SemiPrep Gilson HPLC system: Alltima C18 5 μM 150 mm×22 mm; Buffer A: 95% H₂O, 5% ACN, 0.1% TFA; Buffer B: 20% H₂O, 80% ACN, 0.1% TFA. The fractions containing the pure maleimide containing peptide were pooled and lyophilized to give peptide 2 (33.6 mg, 38%).

Oligonucleotide Synthesis

2′-O-Me phosphorothioate oligonucleotide 3 was assembled on an ÄKTA prime OP-100 synthesiser using the protocols recommended by the supplier. Standard 2-cyanoethyl phosphoramidites and Custom Primer Support (G, 40 μmol/g) were used. Ethylthiotetrazole (ETT, 0.25 M in ACN) was used as coupling reagent and PADS (0.2 M in ACN:3-picoline 1:1 v:v) for the sulfurization step. Oligonucleotide 3 was synthesized on 56 μmol scale. After the oligonucleotide sequence was completed, thiol modifier C6 S—S phosphoramidite (4 eq) was incorporated on line at the 5′ terminus. The crude resin was treated with 40% aqueous ammonia containing 0.1 M DTT at 55° C. for 16 hours. The solid support was filtrated and the filtrate evaporated to dryness. The crude was purified by reversed phase HPLC on a SemiPrep Gilson HPLC system: Alltima C18 5 μM 150 mm×22 mm; Buffer A: 95% H₂O, 5% ACN, 0.1 M (tetraethylamonium acetate (TEAM; Buffer B: 20% H₂O, 80% ACN, 0.1 M TEAA. The fractions containing the pure thiol modified oligonucleotide were pooled and lyophilized. Compound 4 was isolated in 52% yield (29.2 μmol).

Synthesis of Peptide-Oligonucleotide Conjugate LGAQSNF-PS58

Compound 4 (7 mmol) was applied to a PD-10 column pre-equilibrated with phosphate buffer 50 mM, 1 mM EDTA pH=7. The eluted fraction containing the thiol oligonucleotide was directly coupled to maleimide peptide (5 eq, 31 mg) and the reaction was continued at room temperature for 16 hours. The crude was purified by reversed phase HPLC on a SemiPrep Gilson HPLC system: Alltima C18 5 μM 150 mm×22 mm; Buffer A: 95% H₂O, 5% ACN, 0.1 M TEAA; Buffer B: 20% H₂O, 80% ACN, 0.1 M TEAA. The fractions containing the pure conjugate were pooled, NaCl was added and the solvents were evaporated to dryness. Desalting was accomplished through elution on a PD-10 equilibrated with water. After desalting, the pooled fractions were lyophilized to give LGAQSNF-PS58 (25.1 mg, 2.8 μmol, 40% yield)

### Example 2

Materials and Methods

Animals.

Hemizygous DM500 mice—derived from the DM300-328 line (Seznec H. et al)—express a transgenic human DM1 locus, which bears a repeat segment that has expanded to approximately 500 CTG triplets, due to intergenerational triplet repeat instability. For the isolation of immortal DM500 myoblasts, DM500 mice were crossed with H-2K^{b}-tsA58 transgenic mice (Jat P. S. et al). All animal experiments were approved by the Institutional Animal Care and Use Committees of the Radboud University Nijmegen.

Cell Culture.

Immortalized DM500 myoblasts were derived from DM300-328 mice (Seznec H. et al) and cultured and differentiated to myotubes as described before (Mulders S. A. et al).

Oligonucleotides.

AON PS58 ((CAG); SEQ ID NO: 1) was described before (Mulders S. A. et al). The conjugate LGAQSNF was coupled to the 5′ end of AON PS58 or control AON 23 (5′-GGCCAAACCUCGGCUUACCU-3′: SEQ ID NO:3) (Duchenne Muscular Dystrophy (DMD) AON). These AONs were provided by Prosensa Therapeutics B.V. (Leiden, The Netherlands). PS387 ((NAG)₇ wherein N=5-methylcytosine; SEQ ID NO:16) and PS613 ((NAG)₇ XXXX wherein N═C and X is a 1,2-dideoxyribose abasic site attached to the 3′ terminus of the oligo) (SEQ ID NO:17)) were synthesized by Eurogentec (the Netherlands).

Transfection.

All AONs were tested in presence of transfection reagent and LGAQSNF-PS58 was also tested in the absence of transfection reagent. AONs were transfected with polyethyleneimine (PEI) (ExGen 500, Fermentas, Glen Burnie, Md.), according to manufacturer's instructions. Typically, 5 μl PEI solution per μg AON was added in differentiation medium to myotubes on day five of myogenesis at a final oligonucleotide concentration of 200 nM. Fresh medium was supplemented to a maximum volume of 2 mL after four hours. After 24 hours medium was changed. RNA was isolated 48 hours after transfection. LGAQSNF-PS58 was tested following the protocol above with the exception that no transfection reagent was used.

RNA Isolation.

RNA from cultured cells was isolated using the Aurum Total RNA Mini Kit (Bio-Rad, Hercules, Calif.) according to the manufacturer's protocol. RNA from muscle tissue was isolated using TRIzol reagent (Invitrogen). In brief, tissue samples were homogenized in TRIzol (100 mg tissue/mL TRIzol) using a power homogenizer (ultra TURRAX T-8, IKA labortechnik). Chloroform (Merck) was added (0.2 mL per mL TRIzol), mixed, incubated for 3 minutes at room temperature and centrifuged at 13,000 rpm for 15 minutes. The upper aqueous phase was collected and 0.5 mL isopropanol (Merck) was added per 1 mL TRIzol, followed by a 10 min incubation period at room temperature and centrifugation (13,000 rpm, 10 min) The RNA precipitate was washed with 75% (v/v) ethanol (Merck), air dried and dissolved in MilliQ.

Northern Blotting.

Northern blotting was done as described (Mulders S. A. et al). Random-primed ³²P-labeled hDMPK (2.6 kb) and rat Gapdh (1.1 kb) probes were used. Signals were quantified by phospho-imager analysis (GS-505 or Molecular Imager FX, Bio-Rad) and analyzed with Quantity One (Bio-Rad) or ImageJ software. Gapdh levels were used for normalization; RNA levels for control samples were set at 100.

In Vivo Treatment and Muscle Isolation.

Seven month old DM500 mice were anesthetized using isoflurane. The GPS (gastrocnemius-plantaris-soleus) complex was injected on day one and two at the same central position in the GPS muscle with 4 nmoles LGAQSNF-PS58, LGAQSNF-23 or PS58 (SEQ ID NO:1) in a saline solution (0.9% NaCl). In all cases, injection volume was 40 μL. Mice were sacrificed one or three days after final injection and individual muscles were isolated, snap frozen in liquid nitrogen and stored at −80° C.

Quantitative RT-PCR Analysis.

Approximately 1 μg RNA was subjected to cDNA synthesis with random hexamers using the SuperScript first-strand synthesis system (Invitrogen) in a total volume of 20 μL. 3 μL of 1/500 cDNA dilution preparation was subsequently used in a quantitative PCR analysis according to standard procedures in presence of 1× FastStart Universal SYBR Green Master (Roche). Quantitative PCR primers were designed based on NCBI database sequence information. Product identity was confirmed by DNA sequencing. The signal for β-actin and Gapdh was used for normalization. Amplification was performed on a Corbett Life Science Rotor-Gene 6000 using the following 2 step PCR protocol: denaturation for 15 min at 95° C. and 40 cycles of 15 s 95° C. and 50 s 60° C. SYBR Green fluorescence was measured at the end of the extension step (60° C.). After amplification, amplified DNA was dissociated by a melt from 64° C. to 94° C. SYBR Green fluorescence was measured during this step to confirm single amplicon amplification. Serial dilutions of cDNA standards were used to determine the efficiency of each primer set. Critical cycle threshold (Ct) values were determined using Rotor-Gene 6000 Series Software (Corbett Research), the expression of the gene of interest (GOI) was normalized against β-actin and Gapdh and expressed as the ratio to the correspondent control, using formulas according to the ΔΔCt method. The following primers were used:

hDMPK exon 15 (5′)-F;
(SEQ ID NO: 4)
5′- AGAACTGTCTTCGACTCCGGG-3′;
hDMPK exon 15 (5′)-R;
(SEQ ID NO: 5)
5′-TCGGAGCGGTTGTGAACTG-3′;
β-Actin-F;
(SEQ ID NO: 6)
5′- GCTCTGGCTCCTAGCACCAT-3′;
β-Actin-R;
(SEQ ID NO: 7)
5′- GCCACCGATCCACACAGAGT-3′;
Gapdh-F;
(SEQ ID NO: 8)
5′- GTCGGTGTGAACGGATTTG-3′;
Gapdh-R;
(SEQ ID NO: 9)
5′- GAACATGTAGACCATGTAGTTG-3′;

Results

Silencing of hDMPK (CUG)₅₀₀ RNA by LGAQSNF-PS58 in an In Vitro DM1 Model.

Northern blotting revealed a ˜90% silencing of hDMPK transcripts after treatment of DM500 cells with LGAQSNF-PS58 in presence of transfection reagent (PEI), confirming functionality of peptide conjugated PS58. The same level of mutant hDMPK mRNA reduction was found when LGAQSNF-PS58 was added to DM500 cells in absence of transfection reagent indicating that LGAQSNF was responsible for cellular and nuclear uptake of PS58 (FIG. 2).

Intramuscular Injections of LGAQSNF-PS58 Causes Silencing of Expanded hDMPK Transcripts In Vivo.

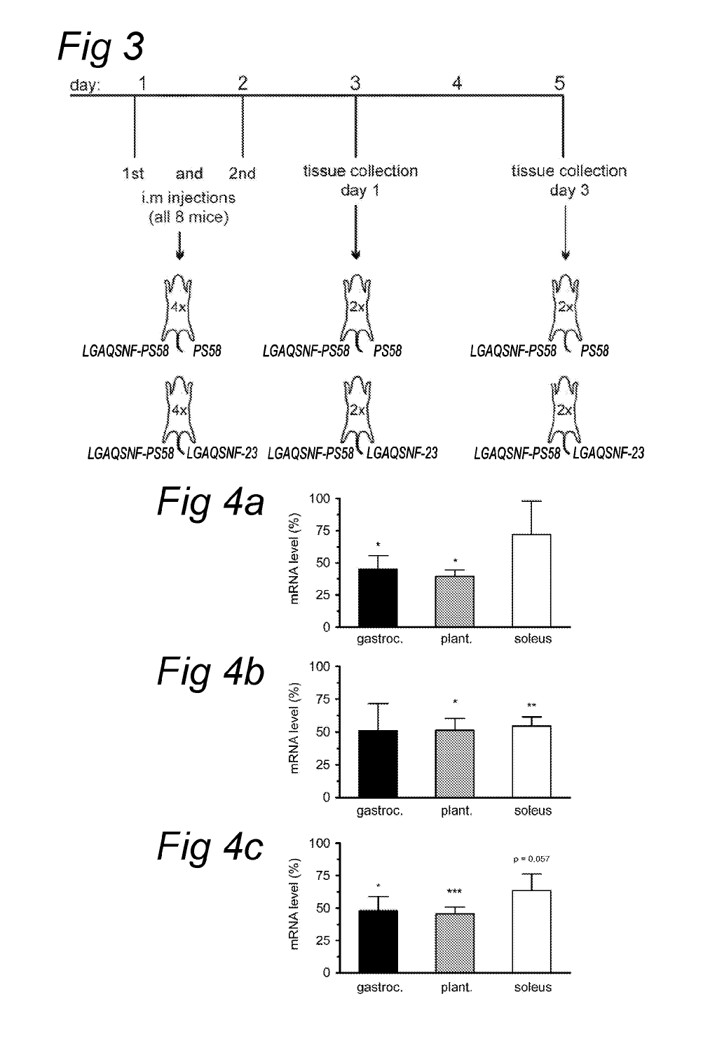

DM500 mice were injected intramuscular (I.M.) in the GPS complex with LGAQSNF-PS58 to reveal functionality of the peptide conjugated version of PS58 in vivo. As control, unconjugated PS58 and LGAQSNF coupled to a DMD control AON 23 (SEQ ID NO: 3) (LGAQSNF-23) were included. Mice were treated for two days with one I.M. injection daily and tissue was isolated on day one or three after the final injection (FIG. 3). Quantitative RT-PCR analysis indicated no statistically significant difference between tissue isolation days so data of both isolation days were grouped. Q-RT-PCR analysis showed a significant reduction of hDMPK mRNA levels after treatment of LGAQSNF-PS58 compared to unconjugated PS58 in both gastrocnemius (55%) and plantaris (60%), and a reduction of 28% was found in soleus (FIG. 4A). A ˜50% silencing of hDMPK (CUG)₅₀₀ levels was found in all individual tissues of the GPS complex after LGAQSNF-PS58 treatment compared to LGAQSNF-23 (FIG. 4B). Because hDMPK transcript levels did not differ significantly between controls, mutant DMPK mRNA levels after LGAQSNF-PS58 treatment were related to both PS58 and LGAQSNF-23 (FIG. 4C). In all individual tissue of the GPS complex tested LGAQSNF-PS58 was responsible for silencing of hDMPK (CUG)₅₀₀ levels not seen after control treatment.

A Compound with an Oligonucleotide Part (CAG)₇ Linked to an Abasic Site Causes a Significant Increase of the Efficiency of Silencing of Expanded hDMPK (CUG)₅₀₀ Transcripts In Vitro Compared to the Efficiency of a Counterpart Compound not Having Said Abasic Site.

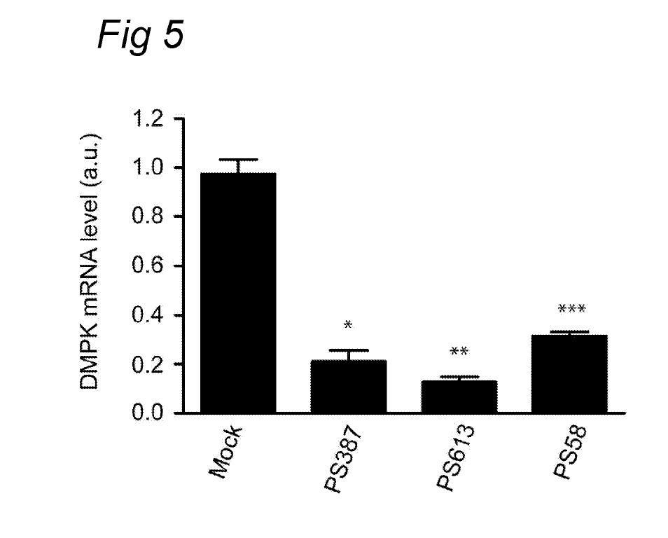

DM500 cells were transfected with 200 nM PS387, PS613 and PS58. Quantitative RT-PCR analysis revealed that both modified AONs (PS387 and PS613) caused a significant silencing of mutant (CUG)₅₀₀ hDMPK transcripts compared to control treated cells (mock). PS58 was included as a positive control (FIG. 5).

### Example 3

### Synthesis of Peptide-2′-O-Me Phosphorothioate RNA Oligonucleotide Conjugate LGAQSNF-(NGA)₇, wherein N═C or 5-methylcytosine, Through a Bifunctional Crosslinker

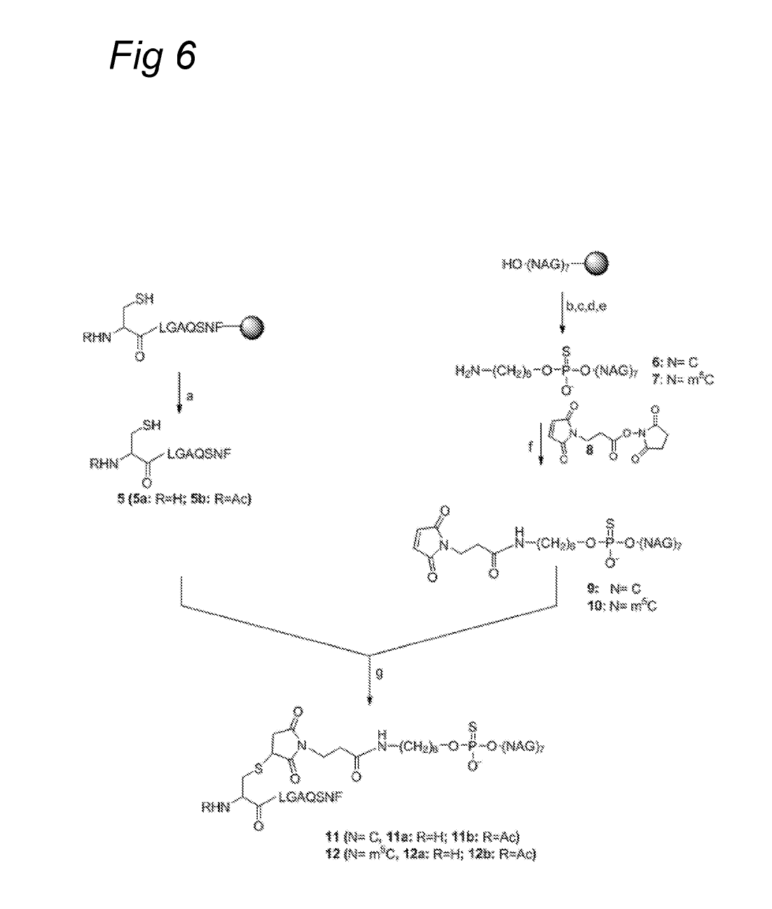

2′-O-Me phosphorothioate (PS)RNA oligonucleotide conjugate LGAQSNF-(NAG)₇, in which N═C (SEQ ID NO: 1) or 5-methylcytosine (m⁵C) (SEQ ID NO: 16) was prepared following the conjugation method depicted in FIG. 6. This conjugation method relies on the coupling of a 5′ amino-modified oligonucleotide (6, 7) to a heterobifunctional crosslinker 8 providing a maleimide-modified oligonucleotide (9, 10), which can be coupled to a thiol-functionalized peptide.

The peptide was assembled on solid support following standard Fmoc peptide synthesis procedures. To provide the peptide with a thiol functionality for enabling coupling of the peptide to the oligonucleotide, a cysteine residue was added to the N-terminus of the peptide. Subsequent acidic cleavage and deprotection afforded peptide 5, whose N-terminus could be prepared as free amine (5a) or as an acetamide group (5b) through capping by acetylation after introduction of the last amino acid.

A monomethoxytrityl (MMT)-protected C6-amino modifier phosphoramidite (Link Technologies) was coupled on-line to the 5′ of the assembled (NAG)₇ 2′-O-Me PS RNA oligonucleotide sequence (N═C or 5-methylcytosine). Cleavage from the solid support and concomitant deprotection of the nucleobases by a two steps basic treatment [diethylamine (DEA) and then ammonia] and subsequent acid treatment to remove the MMT protecting provided amino-modified oligonucleotides 6 and 7.

Reaction of 6 and 7 with β-maleimidopropionic acid succinimide ester (BMPS, 8), a heterobifunctional crosslinker carrying succinimide and maleimide functional groups, afforded maleimide-equipped oligonucleotides 9 and 10, respectively. Peptide-oligonucleotide conjugation was effected through thiol-maleimide coupling of thiol-labeled peptides 5 with maleimide-derived oligonucleotides 9 and 10.

Peptide Synthesis

The peptide sequence CLGAQSNF was assembled on a Tribute peptide synthesizer (Protein Technologies) by standard Fmoc chemistry employing Rink amide MBHA resin (0.625 mmol/g, 160 mg, 100 μmol, NovaBiochem) as described in Example 1. After completion of the peptide synthesis, a final capping step (acetic anhydride (Ac₂O), pyridine) was performed (5b) or omitted (5a). Deprotection and cleavage from the resin was achieved using TFA:H₂O:TIS 95:2.5:2.5 (v:v:v) for 4 h at ambient temperature. The mixture was filtered, precipitated in cold diethyl ether, centrifuged and the supernatant was discarded. Both crude precipitated peptide or RP-HPLC purified peptide were used for the conjugations.

Oligonucleotide Synthesis

2′-O-Me phosphorothioate RNA oligonucleotides (NAG)₇ (wherein N═C (SEQ ID NO:1) or 5-methylcytosine (SEQ ID NO: 16)) were assembled on an ÄKTA Prime OP-100 synthesizer (GE) as described in example 1. After the oligonucleotide sequences were completed, MMT-C6-amino-modifier phosphoramidite was incorporated on-line at the 5′ terminus. The crude resins were then first washed with DEA and then with 29% aqueous ammonia at 55° C. for 16 h. for cleavage and deprotection of base-labile protecting groups. The reaction mixture was filtered and the solvent was removed by evaporation. The oligonucleotides were treated with 80 mL acetic acid (AcOH): H₂O (80:20, v:v) and shaken for 1 h at ambient temperature to remove the MMT group, after which the solvents were removed by evaporation. The crude mixtures were dissolved in 100 mL H₂O and washed with ethyl acetate (3×30 mL). The water layer was concentrated and the residue was purified with RP-HPLC either on a Gilson GX-271 system [C₁₈ Phenomenex Gemini axia NX C-18 5 μm column (150×21.2 mm), buffer A: 95% H₂O, 5% ACN, 0.1 M TEAA; solvent B: buffer B: 20% H₂O, 80% ACN, 0.1 M TEAA. Gradient: 10-60% Buffer B in 20 min] or IEX conditions on a Shimadzu Prominence preparative system [polystyrene Strong Anion Exchange, Source 30Q, 30 μm (100×50 mm) Eluents A: 0.02 M NaOH, 0.01 M NaCl; Eluens B: 0.02 M NaOH, 3 M NaCl. Gradient 0 to 100% B in 40 min]. 70 μL of 100 mM BMPS (8, 7 equiv.) in dimethylsulfoxide (DMSO) was added to 1 μmol amino-modified oligonucleotide (6, 7) in 280 μL phosphate buffer (containing 20% ACN). The reaction mixture was shaken at ambient temperature for 16 h. After filtration over Sephadex G25, 5′-maleimide labeled oligonucleotides 9 and 10 were obtained.

Peptide Oligonucleotide Conjugation

Peptide CLGAQSNF (5a or 5b, 10 equiv.) was added to the 5′-malemide modified oligonucleotide (9 or 10, 1 μmol) in 3.5 mL phosphate buffer and the reaction mixture was shaken at ambient temperature for 16 h. After centrifugation, the supernatant was purified by reversed-phase HPLC on a Prominence HPLC (Shimadzu) [Alltima C₁₈ column (5 μm, 10×250 mm); buffer A: 95% H₂O, 5% ACN, 0.1 M tetraethylammonium acetate (TEAM; buffer B: 20% H₂O, 80% ACN, 0.1 M TEAA]. Fractions containing the pure conjugates were pooled, NaCl was added and the solvents were evaporated. Desalting was accomplished on a Sephadex G25 column equilibrated with water. After desalting, the pooled fractions were lyophilized to provide the final conjugates. LCMS (ESI, negative mode) analysis revealed the correct mass: 10a (N═C, R═H, FIG. 6) Calculated: 8595.3. Found 8595.4, 10b (N=5-methylcytosine, R═Ac) Calculated: 8735.6. Found: 8735.4.

### Example 4

Introduction

The particular characteristics of a chosen AON chemistry may at least in part enhance binding affinity and stability, enhance activity, improve safety, and/or reduce cost of goods by reducing length or improving synthesis and/or purification procedures. This example describes the comparative analysis of the activity of AONs designed to target the expanded (CUG)ₙ repeat in hDMPK (CUG)₅₀₀ transcripts in differentiated DM500 cells in vitro, and includes AONs with 5-methylcytosines (PS387 (SEQ ID NO: 16 and PS389 (SEQ ID NO: 19)) or 2,6-diaminopurines (PS388; SEQ ID NO: 20) versus corresponding AONs (PS147 (SEQ ID NO: 18) and PS58 (SEQ ID NO:1)) without this base modification.

Materials and Methods

Cell Culture.

Immortalized DM500 myoblasts were derived from DM300-328 mice (Seznec H. et al.) and cultured and differentiated to myotubes as described before (Mulders S. A. et al.). In short, DM500 myoblasts were grown on gelatine-coated dishes in high serum DMEM at 33° C. Differentiation to myotubes was induced by placing DM500 myoblasts, grown to confluency on Matrigel, in low serum DMEM at 37° C.

Oligonucleotides.

AON PS58 (CAG)₇) was described before (Mulders S. A. et al.). AONs used were fully 2′-O-methyl phosphorothioate modified: PS147 (NZG)₅ in which N═C and Z=A (SEQ ID NO:18), PS389 (NZG)₅ (SEQ ID NO: 19) and PS387 (NZG)₇ in which N=5-methylcytosine (SEQ ID NO:16) and Z=A, and PS388 (NZG)₅ in which N═C and Z=2,6-diaminopurine (SEQ ID NO:20).

Transfection.

Cells were transfected with AONs complexed with PEI (2 μL per 1 μg AON, in 0.15 M NaCl). AON-PEI complex was added in differentiation medium to myotubes on day five of myogenesis at a final oligonucleotide concentration of 200 nM. Fresh medium was supplemented to a maximum volume of 2 mL after four hours. After 24 hours medium was changed. RNA was isolated 48 hours after transfection.

RNA Isolation.

RNA from cultured cells was isolated using the Aurum Total RNA Mini Kit (Bio-Rad, Hercules, Calif.) according to the manufacturer's protocol.

Quantitative RT-PCR Analysis.

Approximately 1 μg RNA was used for cDNA synthesis with random hexamers using the SuperScript first-strand synthesis system (Invitrogen) in a total volume of 20 μl. 3 μL of 1/500 cDNA dilution preparation was subsequently used in a quantitative PCR analysis according to standard procedures in presence of 1× FastStart Universal SYBR Green Master (Roche). Quantitative PCR primers were designed based on NCBI database sequence information. Product identity was confirmed by DNA sequencing. The signal for β-actin and Gapdh was used for normalization as described in example 2.

Results

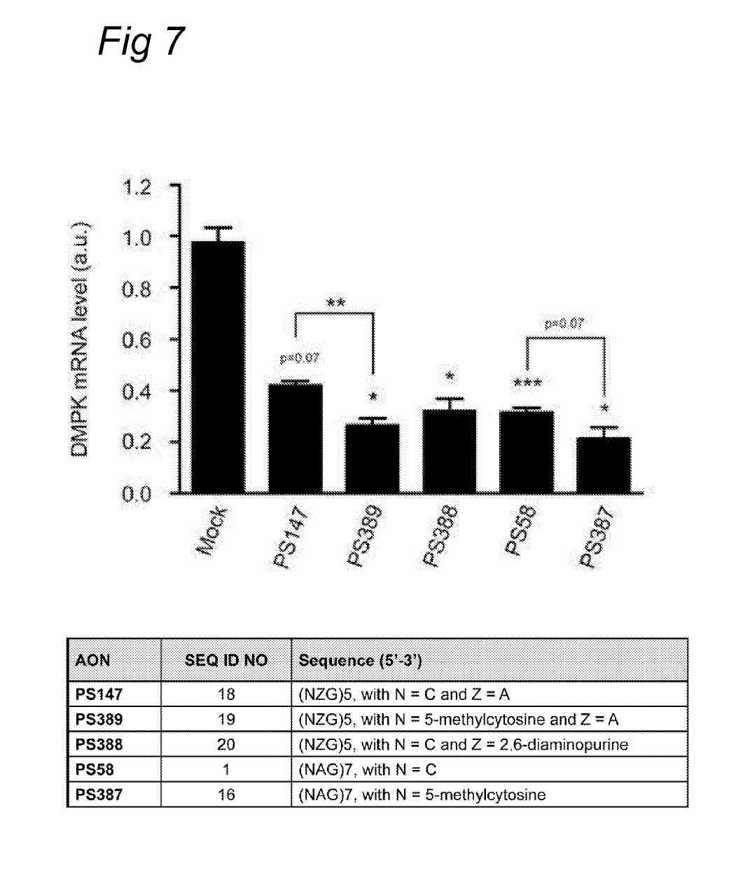

Quantitative RT-PCR analysis demonstrated that all tested AONs induced a significant silencing of hDMPK transcripts after AON treatment when compared to mock treated cells (FIG. 7). The presence of 5-methylcytosines had a significant positive effect on the activity of both the (CAG)₅ (PS147) and (CAG)₇ (PS58) AONs. The presence of 2,6-diaminopurines allowed the shorter (CAG)₅ AON(PS147) to have a similar activity as the longer (CAG)₇ AON(PS58).

### Example 5

Introduction

Myotonic Dystrophy type 1 (DM1) is a complex, multisystemic disease. For AONs to be clinically effective in DM1, they need to reach a wide variety of tissues and cell types therein. A new compound was designed based on conjugation of peptide LGAQSNF to PS58 for improved activity, targeting and/or delivering to and/or uptake by multiple tissues including heart, skeletal and smooth muscle. This example demonstrates its in vivo efficacy on silencing of toxic DMPK transcripts following systemic treatment of DM500 mice.

Materials and Methods

Animals.

Hemizygous DM500 mice—derived from the DM300-328 line (Seznec H. et al.)—express a transgenic human DM1 locus, which bears a repeat segment that has expanded to approximately 500 CTG triplets, due to intergenerational triplet repeat instability. All animal experiments were approved by the Institutional Animal Care and Use Committees of the Radboud University Nijmegen.

Oligonucleotides.

The peptide LGAQSNF was coupled to the 5′ end of AON PS58 (CAG)₇ (SEQ ID NO: 1) or to a control AON (scrambled PS58, 5′-CAGAGGACCACCAGACCAAGG-'3; SEQ ID NO:21), as described in example 1.

In Vivo Treatment.

DM500 mice were injected subcutaneously in the neck region with 100 mg/kg LGAQSNF-PS58 or LGAQSNF-control AON. Injections were given for four consecutive days and tissue was isolated one day after the final injection.

RNA Isolation.

RNA from tissue was isolated using TRIzol reagent (Invitrogen). In brief, tissue samples were homogenized in TRIzol (100 mg tissue/mL TRIzol) using a power homogenizer (ultra TURRAX T-8, IKA labortechnik). Chloroform (Merck) was added (0.2 mL per mL TRIzol), mixed, incubated for 3 minutes at room temperature and centrifuged at 13,000 rpm for 15 minutes. The upper aqueous phase was collected and 0.5 mL isopropanol (Merck) was added per 1 mL TRIzol, followed by a 10 min incubation period at room temperature and centrifugation (13,000 rpm, 10 min). The RNA precipitate was washed with 75% (v/v) ethanol (Merck), air dried and dissolved in MilliQ.

Quantitative RT-PCR Analysis.

Approximately 1 μg RNA was subjected to cDNA synthesis with random hexamers using the SuperScript first-strand synthesis system (Invitrogen) in a total volume of 20 μL. 3 μl of 1/500 cDNA dilution preparation was subsequently used in a quantitative PCR analysis according to standard procedures in presence of 1× FastStart Universal SYBR Green Master (Roche). Quantitative PCR primers were designed based on NCBI database sequence information. Product identity was confirmed by DNA sequencing. The signal for β-actin and Gapdh was used for normalization as described in example 2.

Results

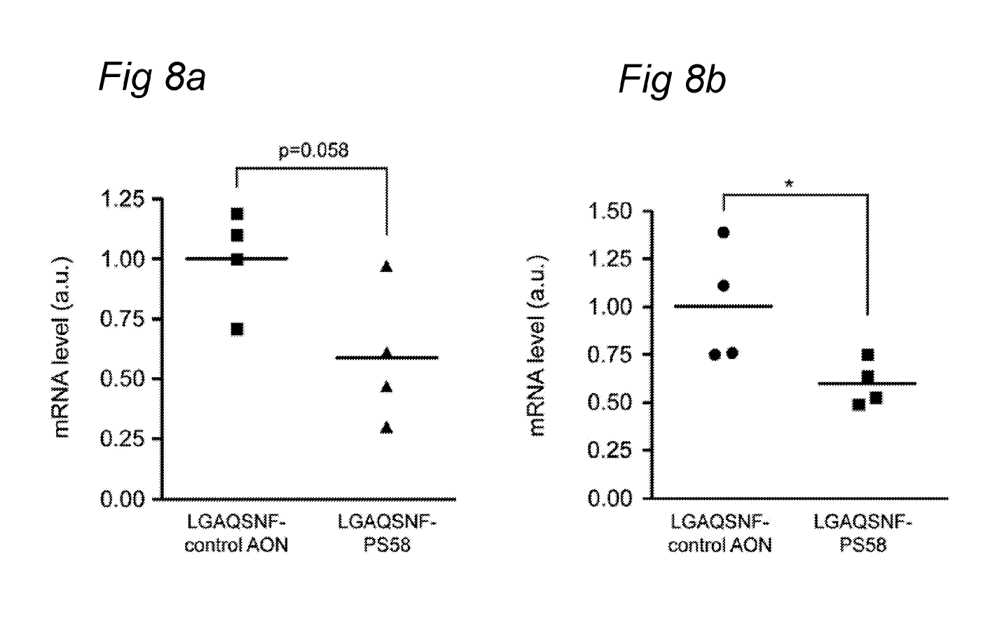

Quantitative RT-PCR analysis demonstrated that systemic treatment with LGAQSNF-PS58 resulted in a significant reduction of expanded hDMPK (CUG)500 transcripts in DM500 mice when compared to mice treated with LGAQSNF-control AON. In both gastrocnemius and heart muscles an overall ˜40% reduction of hDMPK levels was found (FIG. 8), indicating that the peptide LGAQSNF promoted delivery and/or activity of PS58 in two target organs affected in DM1.

### Example 6

Introduction

Myotonic Dystrophy type 1 (DM1) is a complex, multisystemic disease. For AONs to be clinically effective in DM1, they need to reach a wide variety of tissues and cell types therein. A new compound was designed based on conjugation of peptide LGAQSNF to PS58 for improved activity, targeting and/or delivering to and/or uptake by multiple tissues including heart, skeletal and smooth muscle. This example demonstrates its in vivo efficacy in HSA^{LR} mice. These mice, expressing a toxic (CUG)250 repeat in a human skeletal actin transgene, not only show molecular deficits similar to DM1 patients but also display a myotonia phenotype.

Materials and Methods

Animals.

Homozygous HSA^{LR} mice (line HSA^{LR}20b) express 250 CTG repeats within the 3′ UTR of a transgenic human skeletal α-actin gene (Mankodi A. et al.). HSA^{LR} mice develop ribonuclear inclusions, myotonia, myopathic features and histological muscle changes similar to DM1. All animal experiments were approved by the Institutional Animal Care and Use Committees of the Radboud University Nijmegen.

Oligonucleotides.

The peptide LGAQSNF was coupled to the 5′ end of AON PS58 (CAG)₇ (SEQ ID NO:1) as described in example 1.

In Vivo Treatment.

HSA^{LR} mice were injected subcutaneously in the neck region with LGAQSNF-PS58 for five consecutive days at a dose of 250 mg/kg, and compared to control mice that received saline injections only. EMG measurements were performed on a weekly base and tissue was isolated four weeks after the first injection.

EMG.

EMG was performed under general anaesthesia. A minimum of 5-10 needle insertions were performed for each muscle examination. Myotonic discharges were graded on a 4-point scale: 0, no myotonia; 1, occasional myotonic discharge in less than 50% of needle insertions; 2, myotonic discharges in greater than 50% of needle insertions; 3, myotonic discharge with nearly every insertion

RNA Isolation.

RNA from tissue was isolated using TRIzol reagent (Invitrogen). In brief, tissue samples were homogenized in TRIzol (100 mg tissue/mL TRIzol) using a power homogenizer (ultra TURRAX T-8, IKA labortechnik). Chloroform (Merck) was added (0.2 mL per mL TRIzol), mixed, incubated for 3 minutes at room temperature and centrifuged at 13,000 rpm for 15 minutes. The upper aqueous phase was collected and 0.5 mL isopropanol (Merck) was added per 1 mL TRIzol, followed by a 10 min incubation period at room temperature and centrifugation (13,000 rpm, 10 min). The RNA precipitate was washed with 75% (v/v) ethanol (Merck), air dried and dissolved in MilliQ.

Northern Blotting.

RNA was electrophoresed in a 1.2% agarose-formaldehyde denaturing gel loaded with one mg RNA per lane. RNA was transferred to Hybond-XL nylon membrane (Amersham Pharmacia Biotech, Little Chalfont, UK) and hybridized with 32P-end-labeled (CAG)₉ or mouse skeletal actin-specific (MSA) oligos. Blots were exposed to X-ray film (Kodak, X-OMAT AR). Quantification of signals was done by phospho-imager analysis (GS-505 or Molecular Imager FX, Bio-Rad) and analyzed with Quantity One (Bio-Rad) or ImageJ software. MSA levels were used for normalization.

Semi-Quantitative RT-PCR Analysis.

Approximately 1 μg RNA was used for cDNA synthesis with random hexamers using the SuperScript first-strand synthesis system (Invitrogen) in a total volume of 20 μL. One μl of cDNA preparation was subsequently used in a semi-quantitative PCR analysis according to standard procedures. In RT-control experiments, reverse transcriptase was omitted. Product identity was confirmed by DNA sequencing. PCR products were analyzed on 1.5-2.5% agarose gels, stained by ethidium bromide. Quantification of signals was done using the Labworks 4.0 software (UVP Biolmaging systems, Cambridge, United Kingdom). For analysis of alternative splicing, embryonic (E):adult (A) splice ratio was defined as embryonic form signal divided by adult form signal in each sample. Splice ratio correction illustrates the effect of LGAQSNF-PS58 treatment on alternative splicing (i.e., Sercal, Ttn and Clcn1). The following primers were used:

| (SEQ ID NO: 22) | |
|---|---|
| Sercal-F; | 5′- GCTCATGGTCCTCAAGATCTCAC-3′ |
| (SEQ ID NO: 23) | |
| Sercal-R; | 5′- GGGTCAGTGCCTCAGCTTTG-3′ |
| (SEQ ID NO: 24) | |
| Ttn-F; | 5′- GTGTGAGTCGCTCCAGAAACG-3′ |
| (SEQ ID NO; 25) | |
| Ttn-R; | 5′- CCACCACAGGACCATGTTATTTC-3′ |
| (SEQ ID NO: 26) | |
| Clcn1-F; | 5′- GGAATACCTCACACTCAAGGCC-3′ |
| (SEQ ID NO: 27) | |
| Clcn1-R; | 5′- CACGGAACACAAAGGCACTGAATGT-3′ |

Results

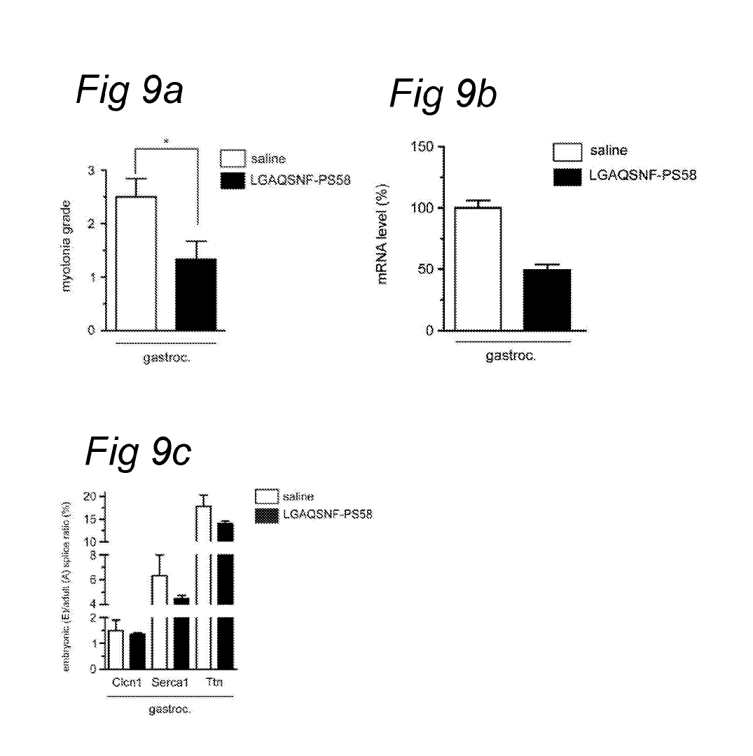

Four weeks after the first injection, EMG measurements in the gastrocnemius muscles revealed a significant, but mild, reduction in myotonia in LGAQSNF-PS58 treated mice when compared to saline-treated mice (FIG. 9A). This reduction in myotonia was paralleled by a ˜50% reduction in toxic (CUG)₂₅₀ transcript levels (FIG. 9B), and a shift in splicing pattern form an embryonic-like (E) to normal-adult (A) mode for Clcn1, Serca 1 and Ttn transcripts (FIG. 9C) in the gastrocnemius muscles. These results indicate that the peptide LGAQSNF indeed promoted delivery and/or activity of PS58 in muscle in vivo, both on molecular and phenotypic level.

### Example 7

Introduction

This example again demonstrates the in vivo efficacy of LGAQSNF-PS58 in HSA^{LR} mice. The mice were here treated for a prolonged period of time. Silencing of toxic (CUG)₂₅₀ transcripts and splicing pattern shifts of downstream genes were monitored and compared to those in saline-treated mice.

Materials and Methods

Animals.

Homozygous HSA^{LR} mice (line HSA^{LR}20b) express 250 CTG repeats within the 3 'UTR of a transgenic human skeletal α-actin gene (Mankodi A. et al.). HSA^{LR} mice develop ribonuclear inclusions, myotonia, myopathic features and histological muscle changes similar to DM1. All animal experiments were approved by the Institutional Animal Care and Use Committees of the Radboud University Nijmegen.

Oligonucleotides.

The peptide LGAQSNF was coupled to the 5′ end of AON PS58 (CAG)₇ (SEQ ID NO:1) as described in example 1.

In Vivo Treatment.

HSA^{LR} mice that received eleven subcutaneous injections of 250 mg/kg LGAQSNF-PS58 in the neck region in a four weeks period were compared to mice that were injected with saline only. Thirty-two days after the first injection all mice were sacrificed and tissue was isolated.

RNA Isolation.

RNA from tissue was isolated using TRIzol reagent (Invitrogen). In brief, tissue samples were homogenized in TRIzol (100 mg tissue/mL TRIzol) using a power homogenizer (ultra TURRAX T-8, IKA labortechnik). Chloroform (Merck) was added (0.2 mL per mL TRIzol), mixed, incubated for 3 minutes at room temperature and centrifuged at 13,000 rpm for 15 minutes. The upper aqueous phase was collected and 0.5 mL isopropanol (Merck) was added per 1 mL TRIzol, followed by a 10 min incubation period at room temperature and centrifugation (13,000 rpm, 10 min). The RNA precipitate was washed with 75% (v/v) ethanol (Merck), air dried and dissolved in MilliQ.

Northern Blotting.

RNA was electrophoresed in a 1.2% agarose-formaldehyde denaturing gel loaded with one mg RNA per lane. RNA was transferred to Hybond-XL nylon membrane (Amersham Pharmacia Biotech, Little Chalfont, UK) and hybridized with 32P-end-labeled (CAG)₉ or mouse skeletal actin-specific (MSA) oligos. Blots were exposed to X-ray film (Kodak, X-OMAT AR). Quantification of signals was done by phospho-imager analysis (GS-505 or Molecular Imager FX, Bio-Rad) and analyzed with Quantity One (Bio-Rad) or ImageJ software. MSA levels were used for normalization.

Semi-Quantitative RT-PCR Analysis.

Approximately 1 μg RNA was used for cDNA synthesis with random hexamers using the SuperScript first-strand synthesis system (Invitrogen) in a total volume of 20 μL. One μl of cDNA preparation was subsequently used in a semi-quantitative PCR analysis according to standard procedures. In RT-control experiments, reverse transcriptase was omitted. Product identity was confirmed by DNA sequencing. PCR products were analyzed on 1.5-2.5% agarose gels, stained by ethidium bromide. Quantification of signals was done using the Labworks 4.0 software (UVP Biolmaging systems, Cambridge, United Kingdom). For analysis of alternative splicing, embryonic (E):adult (A) splice ratio was defined as embryonic form signal divided by adult form signal in each sample. Splice ratio correction illustrates the effect of LGAQSNF-PS58 treatment on alternative splicing (i.e., Serca1, Ttn and Clcn1). The following primers were used:

| (SEQ ID NO: 22) | |
|---|---|
| Sercal-F; | 5′- GCTCATGGTCCTCAAGATCTCAC-3′ |
| (SEQ ID NO: 23) | |
| Sercal-R; | 5′- GGGTCAGTGCCTCAGCTTTG-3′ |
| (SEQ ID NO: 24) | |
| Ttn-F; | 5′- GTGTGAGTCGCTCCAGAAACG-3′ |
| (SEQ ID NO: 25) | |
| Ttn-R; | 5′- CCACCACAGGACCATGTTATTTC-3′ |
| (SEQ ID NO: 26) | |
| Clcn1-F; | 5′- GGAATACCTCACACTCAAGGCC-3′ |
| (SEQ ID NO: 27) | |
| Clcn1-R; | 5′- CACGGAACACAAAGGCACTGAATGT-3′ |

Results

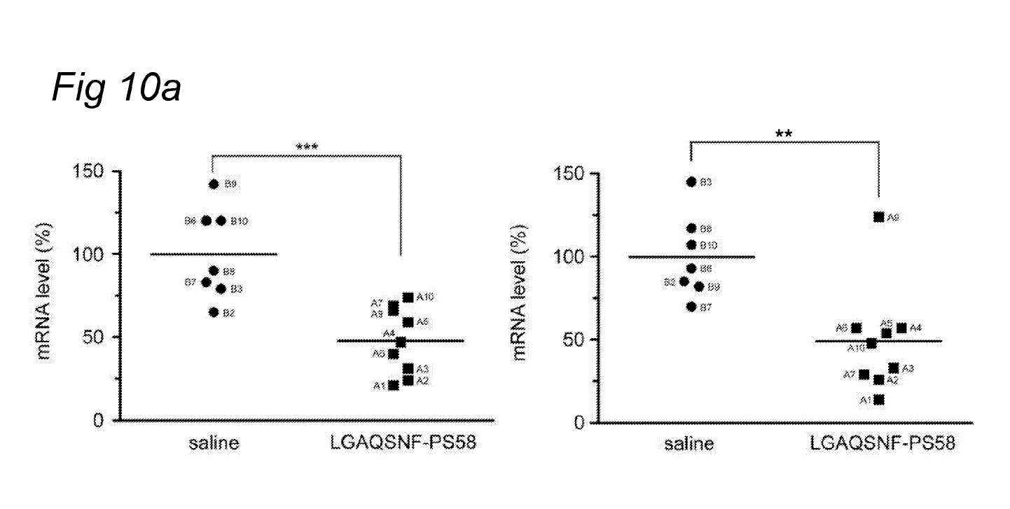

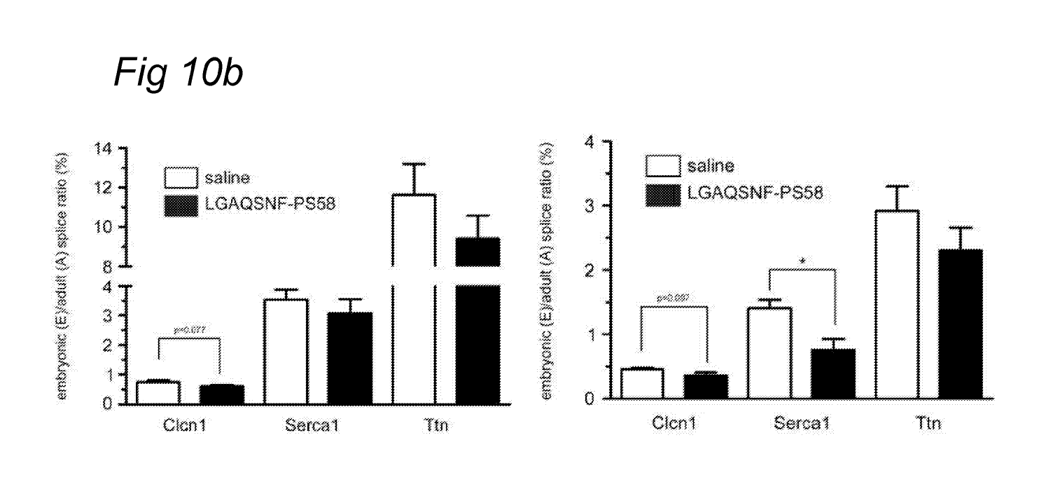

Thirty-two days after the first injection, HSA^{LR} mice were sacrificed and tissue was isolated. Northern blotting showed a significant reduction in toxic (CUG)₂₅₀ levels both in the gastrocnemius (FIG. 10a, left graph) and tibialis anterior (FIG. 10a, right graph) muscles of LGAQSNF-PS58 treated mice when compared to those in saline-treated mice. In both muscle groups an average (CUG)₂₅₀ reduction of ˜50% was found. This reduction was paralleled by a shift from an embryonic-like (E) to normal-adult (A) splicing pattern for Clcn1, Serca 1 and Ttn transcripts both in gastrocnemius (FIG. 10b, left graph) and tibilais anterior (FIG. 10b, right graph) muscles. These results again indicate that the peptide LGAQSNF promotes delivery and/or activity of PS58 in muscle in vivo.

TABLE 1
Oligonucleotides and peptides used in experi-
mental part

| | | SEQ ID |
|---|---|---|
| Name | AON Sequence (5′→3′) | NO |
| PS58 | (CAG)₇ | 1 |
| PP08 | LGAQSNF | 2 |
| “23” | GGCCAAACCUCGGCUUACCU | 3 |
| control | | |
| AON | | |
| PS387 | (NAG)₇ | 16 |
| | N = 5-methylcytosine | |
| PS613 | (NAG)₇XXXX | |
| | N = C | 17 |
| | X = 1,2-dideoxyribose abasic site | |
| PS147 | (NZG)₅ | 18 |
| | N = C and Z = A | |
| PS389 | (NZG)₅ | 19 |
| | N = 5-methylcytosine and Z = A | |
| PS388 | (NZG)₅ | 20 |
| | N = C and Z = 2,6-diaminopurine | |
| scrambled | CAGAGGACCACCAGACCAAGG | 21 |
| PS58 | | |

### REFERENCE LIST

Braida C. et al, Human Molecular Genetics, (2010), vol9: 1399-1412.
Ede, N. J.; Tregear, G. W.; Haralambidis, J. Bioconj. Chem. 1994, 5, 373-378.
Harper PS (1989) Myotonic Dystrophy (Saunders, W. B., Philadelphia).
Hébert et al. BMC Musculoskeletal Disorders 2010, 11:72.
Hongquing D. et al., Nature structural & molecular biology 2010; 17: 141-142
Januario et al, Disability and Rehabilitation, 2010; 32(21): 1775-1779
Jat P S, et al. (1991). Proc Natl Acad Sci USA 88:5096-5100.
Kumar L, Pharm. Technol. 2008, 3, 128.
Mahant et al, Neurology. 2003; 61(8):1085-92
Mankodi A. et al., The journal of general physiology 2007; 129(1):79-94.
Mulders S A, et al. (2009) Proc Natl Acad Sci USA 106:13915-13920.
Nakamura et al, Journal of the Neurological Sciences 278 (2009) 107-111
Remington: The Science and Practice of Pharmacy, 20th Edition. Baltimore, Md.: Lippincott Williams & Wilkins, 2000.
Seznec H, et al. (2000). Hum Mol Genet. 9:1185-1194.
Taneja K L et al., Journal of cell biology 1995; 128: 995-1002
Tones C. et al., Journal of neurological sciences. 1983; 60:157-168
Trouillas P. et al, J. Neurol. Sci., 1997:145:205-211
Walker, 2007 LANCET 369; p. 218-228
Wiles, et al, J Neurol Neurosurg Psychiatry 2006; 77:393-396

1. A compound comprising the oligonucleotide sequence (NAG)ₘ, wherein N is C or 5-methylcytosine and at least one occurrence of N is 5-methylcytosine and/or at least one occurrence of A comprises a 2,6-diaminopurine nucleobase modification, and wherein m is an integer from 4 to 15.

2. A compound according to claim 1, wherein said compound consists of said oligonucleotide sequence.

3. A compound according to claim 1, wherein said compound lacks an inosine nucleotide.

4. A compound according to claim 1, wherein all occurrences of N are 5-methylcytosine.

5. A compound according to claim 1, wherein all occurrences of A comprise a 2,6-diaminopurine nucleobase modification.

6. A compound according to claim 1, comprising SEQ ID NO:16, 17, 19 and/or 20.

7. A compound according to claim 6, wherein said compound consists of SEQ 16, 17, 19, and/or 20.

8. A compound according to claim 6, comprising SEQ ID NO:16 and having a length of 21, 22, 23, 24, 25, 26, 27, 28, 29, 30 nucleotides.

9. A compound according to claim 1, further comprising a peptide comprising LGAQSNF linked to said oligonucleotide comprising (NAG)ₘ in which N is C or 5-methylcytosine, and wherein m is 4, 5, 6, 7, 8, 9, 10, 11, 12, 13, 14, or 15.

10. A compound according to claim 1, wherein the length of the oligonucleotide comprising (NAG)ₘ, in which N is C or 5-methylcytosine, is from 12 till 45 nucleotides.

11. A compound according to claim 1, wherein the oligonucleotide comprises at least one modification, wherein said modification is selected from the group consisting of a backbone modification, a sugar modification and a base modification, when compared to an RNA-based oligonucleotide.

12. A compound according to claim 11, wherein said modification is selected from the group consisting of 2′-O-methyl phosphorothioate, morpholino phosphorodiamidate, locked nucleic acid and peptide nucleic acid.

13. A compound according to claim 12, wherein the oligonucleotide is a 2′-O-methyl phosphorothioate oligonucleotide.

14. A compound according to claim 9, wherein said oligonucleotide comprises at least one 2,6-diaminopurine, 2-thiouracil, 2-thiothymine, 5-methyluracil, 5-methylcytosine, thymine, 8-aza-7-deazaguanosine, and/or hypoxanthine.

15. A compound according to claim 1, wherein 1-10 abasic monomers are present at a free terminus of said oligonucleotide, said abasic monomer preferably chosen from the group consisting of 1-deoxyribose, 1,2-dideoxyribose, and/or 1-deoxy-2-β-methylribose.

16. A compound according to claim 15, wherein 4 monomers of 1-deoxyribose, 1,2-dideoxyribose, and/or 1-deoxy-2-O-methylribose are present at the 3′ terminus of the oligonucleotide part, preferably wherein the oligonucleotide or oligonucleotide part is (NAG)₇, in which N is C or 5-methylcytosine.

17. A compound according to claim 9, wherein the peptide is linked to the oligonucleotide via a linker comprising a thioether moiety.

18. A compound represented by H—(X)ₚ—(NAG)ₘ-(Y)_{q}—H, wherein N is C or 5-methylcytosine and at least one occurrence of N is 5-methylcytosine and/or at least one occurrence of A comprises a 2,6-diaminopurine nucleobase modification;
m is an integer from 4 to 15;
each occurrence of X and Y is, individually, absent an abasic monomer or a nucleotide; and
p and q are each individually an integer from 0 to 10.

19. A pharmaceutically acceptable composition comprising a compound as defined in claim 1.

20. An in vitro method for the reduction of the number of CUG repeats in the transcript of a diseased allele of gene DM1/DMPK, SCA8 or JPH3 in a cell comprising contactin said cell in vitro with a compound as defined in claim 1 or a pharmaceutically acceptable composition thereof, in an amount effective to achieve said reduction.

21. A method for alleviating one or more symptom(s) and/or characteristic(s) and/or for improving a parameter of dystrophy type 1 (DM1), spino-cerebellar ataxia 8 and/or Huntington's disease-like 2 caused by expansion of CUG repeats in the transcripts of DM1/DMPK, SCA8 or JPH3 genes in an individual, the method comprising administering to said individual a compound as defined in claim 1, or a pharmaceutical composition thereof.


SEQUENCE LISTING (27 sequences)

SEQ ID NO: 1 - RNA, 21 nt, Artificial
  oligonucleotide
cagcagcagcagcagcagcag

SEQ ID NO: 2 - protein, 7 aa, Artificial
  peptide
LGAQSNF

SEQ ID NO: 3 - RNA, 20 nt, Artificial
  oligonucleotide
ggccaaaccucggcuuaccu

SEQ ID NO: 4 - DNA, 21 bp, Artificial
  primer
agaactgtcttcgactccggg

SEQ ID NO: 5 - DNA, 19 bp, Artificial
  primer
tcggagcggttgtgaactg

SEQ ID NO: 6 - DNA, 20 bp, Artificial
  primer
gctctggctcctagcaccat

SEQ ID NO: 7 - DNA, 20 bp, Artificial
  primer
gccaccgatccacacagagt

SEQ ID NO: 8 - DNA, 19 bp, Artificial
  primer
gtcggtgtgaacggatttg

SEQ ID NO: 9 - DNA, 22 bp, Artificial
  primer
gaacatgtagaccatgtagttg

SEQ ID NO: 10 - DNA, 12841 bp, Homo sapiens
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaa

SEQ ID NO: 11 - DNA, 32541 bp, Homo sapiens
atccttcacctgttgcctggctagagttgtctggctccactttgagctcttgcagaaccagccctttttcgtgtggtccaggaaagtccatgcctggcaccacctcctcctctagtgactccacgtagaagagagtcctggctggctgctgagtgccctgcccaggagccccttgctgcagcctcgtggcaactggaagcagggtgccattcagcggattgaaggaagaggaggaagaggacggggaggacgatgaagaggaagaggaggaaggcttcttccagaaagtgctcacaccgcttctctcttggcttttgagcaggcgactctggctgggtccccagtgctcaaagctgccactgccgtcctgttgcaggcagcctccccccgccgggccgccggtggaaggagacgggtggctgaagagtttccagcggagtcgcagaatgtgcttcacatcgaagtcttttcgcccagagcctgacatgctttacgcacagaaggcaaaaggctggcagctcacgcaggagtaggctggtcagcaggtgggggaggacagcggggcccgggggcggaggaagaggcgggatgcgccctctgcacccctagagccagaagacgctaggtgggctgcgcgctctgccaggcgaaggctggagcgcagacggcaaagccgcgcgtttcagccgtggtcgggtccgcaggacctgggcgtggggacaccaccaggcaggagcagaggcaggactgggacgccaaaagctgagaatcctcgatgcccgcgcgagagccccgtgttatggcgaggtgggacaacccttaggctggagatgcgcgagggagggaggtctgagcgctccgaagctccggaggcggctgcagctggatacacctcactgggatgcgcttgtggcagagccttagtagggaaggtggtggcgttcttgtccttgcagctcagagttcagtgtctggagagcgcagagagaaagagccccaagtctcgaggaagcgtacccctcgccagatctcttggtgcacctgcgcccctgtccctggccttttcgaggatgccccgatagcctgccgggtggctctgagaaagtcaattgctttctgcaatgccagaagaggtggttttatatagtcagtttgtaaaagagaaaaatagatattctagcgcatatagggaggcaaaagaaaaagcccgcctgtgaagctgtcaaggtcctcacagtacaattttctctctgcctcagcgcctcctcctcccctgtaagtgacgcagatgtgcactggggcctatacggagagatggagggagggagggagggagggaaggcttcaatcttctttatgcaagtgagactgctgctcttattgtccccttgcgtgggtcttgtcaccttttttcaccctccttcctcgctacattactgtgtagcaatacaaggaaagaaaacaggcattatgcttttgcatcagtaaacaccacacatgaatcatggcagtgtaaacttaataggctgccatcagtttgcaggttgcaggttaatggcttgaattatttaatgaaccgcgtcttttaacctttagccttagtttctctgtagtgagacacagaagaaacggatgctggcgtcaaaatgtgacgatgccaccatcctgacacttacattgtgattctgctattaaggcacccaggcactgcttcactcctggggccagaggcgtacggtagaaatttgaagatgggcatctgaacacgggggacgggttttgcgaaatcagtgcatttggtgtgtagcctcggaatgaaagctgttatcagtgtaaatcaagaatggaataaaatgataaaagagaatttcaaggtataaccttctagtacataaatcattgtatactatgcatggaagagtttaggcaataaatgagaatagccgaaggctacttactatcttgcaaataggcgtttggagaagatgtgttttccccttatatctatacttagaatggtgaggattgcgatcactttcagtacatgactacttggaatgttttaacctttcctttaatgagccattaagatagaacccagtagtctttgttgaaggcaaagggaccagaaatgcaatgtgttccctaacttaaccaagaatatggttgttgtccattcatgaaattcacagtttcagtaacccccaacagagcataggaatcaaccactgaagtgcagttaggctactgctcagttacccccaaggttgagctgtaaacgtaaccagaaaccaaagctaaatttggagtccttaggcaaagttggacgctttaataaaataagaaattaagtgcacaaaagacctatttttaggggaataaaaatcactatagtgcttttaaaatattgatttatgccacaacagctggactgcaatcccttgtgccttgatttttctctcagtattcagtgattttttgtttgttttttgtttattaagcaaaaaaccctttaaagccaacaatggtgacagatcatttttatccaacaatggaatcaagtaagcatttcccttagggaagaaaatagtttatgaaattacacatcacaaacaggacagaggaaactttccatcagaaataatgttgaaagttgaagtgcatgtaattaatttatatagaaattagtgcttcatggattatctcacttgatttatttaaaataatataaaataacataagtcaagtctctgtatctatatttttcattcaccattcacaaactgtaatataaaatttatttgttattctaaaatttaattttgcagttcctttttctctgcaatactaataagaatattccacagttaaaagttatatgtttaaaggacaatattttatatgacccaaaggagatataacattaatttgtggtataaaggaacatgaattaaaaatattgtcacatttagcagatggtgaataaagcctatttgttagtcttgtagtaacatttagtatgtttaaagcactgccagagccaattatgtaaatataggtacctttctatatttgtcaatcttaatacaatatataagtttgaaaaaatcactcttcttgggaatatatttaaataaaattcaactttattatcaaatgaaaatatttcgaatttaagaattaaattacgcatgaagttatgttaaactgaaatgactgacaaagatgattatattattttcacttgattgttaccatactacagtgttgtagaaaaggaaggagaaaataaaacaagaaaaaaaaatggaagaaagagaggaaagtgagaaggacgagaagagaaggagaaaaaagaaagagggagagactgcgagagagggaaagacagagacaggaaaggaggaaggaaaataatttggttggatattatggcttaacctggcatattagataaaatccttgaaaaatatactttgtaaaatactgcactaattaatttatctattattcaaaaataaaaaactttgattgatcacttaggtccaatgtaaactagtaacaaatatgaaggaattggtcccatgcagcttgctggaagaacccacaaaagaggactgtttaaccagccataagtaataattgacttgtgagtcaattaaattaagatttaagataaccaagcttataggcaagaagacattgacataataaagaccacttttaacaatcccaacaaacacactttgttttaaagacagaaactatttaattattctaacactattgaattaaattgaactgtcagttgaatcaatcagagtctttcaaatgaatgtccatatggttgccatagtttcctgaaacactaagcaaaaaggttaatcgggatacagaaaatctgtagaatgctcaagtatttactgtaatacacaaatacaaaactcttcgcataagtattaagtcaagcatgggtgtaaaatgtatcacactgaaagtttctacaactcaaatatttaaacaataagcaaaattggctttggatctgttttagtccattatctgttacttgtaatagaaaacctgaaggtgggtaacttataaagataaggaatttatttattatagttatggaggctgagaagtctaagttctagaggctgcatctggtgaaggtcttcttcctggtggggactctctgcagagtcctggagctgcacaggcatcatatggcaatggggttaagcatgctgtctcaggtccctcttccttttcttataaagccactaatcccacttccatgataacccattaatacattaattcttgaggttctgccctcgagacccaatcacctcctaaatgcccccctctcaatattccacaacagagattaaatttcaacatgaattttggaggggacaaaaattcataccatagcagaattcaagaattttctagactgaaagcttctcacttttagtgactattttctaaacaagcacaattaaatgtatacttttccaggaaaagcaattttttttgtatctttttggcaggccttctgcaatgcctagaaatttcttctacatgataatactcaacaaaaacatttgaatggctaattatgctcaaatttgaaagttcatgccttaaaatggctcctgtgattttgtctttgattaattttaataagatgtatttatatacattttattagtcaatttttgcactgctataaagacattcctgagactgggtaatttataaagaaaataagtttaatcagctcacagttctgcagagtgtacagccttctgcttctggggaggaggcctgaggaaacttataatcatggcagaaggcaaaggggaagcaggcacgtcctacgtggctggagcaggaagaagagagagctaatgtaaaggtgccacacactttcaaataaccagatctcatgagagttctatcatgagaacagaaagatggacgcccacccttatgattccatcacctcacactaggatcctactctagcaccggggattataatttgacatgagatttgggtggggacacagagccaaaccatatcatacattaatataaaatattatttaaaattagagaataccatttaaaataattatatagaacttttaatgtatacattaaataaaaattgttttgcagtgactttttttcccaaaatgattgaaacataatatcatatagcactaatgctaagttatcccatgcataatcaagggttcctacctcttaaaacaaatacacataaccattcatggtctattaatcttcattacagtatagttatagtttacttaagtgtcaattaagtgactgcttaataaacatttgtaggtattcattgatccccaggggccattttaatccactcaacctccttggtttatttgaccttttgatttagatctggcaatacatttgtatagttgaggaattcttatataaattgctgagttaatttcattagtatgtgaattcaaaatggcattaaaatagtgaatttctattaatgatgagccaggttttatttgtgtgtctttttatttcccaaaattctgtgcagaatattgacagtggtttatgaacattcattattttcacttttgtgctgggtcctgtctggttaatcacactgctgagggactttatatatataattttttttttttttttttttgagatggagtctcgctctgtcgccgaggctggagtgcagtggcgtgattcctgctcactgcaaccttcacctcctggattcaagtgattctcttgcctcagcctcccaagtagctgggactacaggcaccaccaccacacccagctaattttcgtatttttagtagagacggggtttcaccatattggccaggttggtctcaaactcctgacgctgtgatcctcacatcttggcctcccaaagtgctgggattacaggcataagccacagcgcctggcctatgatgtgatttttaaaacactttctatcccttttatgaaaatatttatagattgaaaacaaatatatgtagacatatagatatagataaatgtaatatacatatacatttaataagtatttcaaggatattttctaatgagtaaaacacatttgatggcatgatatcctattttgtataacactagagtctttaaaaaagaaaactgcttaactaatacttatttacaaagggttaagatttccctagcattaaaaaagtaatttcattattttgtatattgcactgaaatttgcctgtatcactgtggggtaaggtgagtcccctgaaactttaatcaggatgatcaaaataaaatagtaagattatagtttataaaatacagatttggaagttataggagtgtaagatattcatataaatatagataaaatagtataactttgtggatattttattttctgttgtctcttttgagacatgggtttttggagactttaaaatattttctcaaatcaggttactacatttctctttccatcttgagacatatttgccaaaactttatattttcattcacctgcattattaccagatcttttaaaaatctaggacaaaaatgcatggctttaaaagctaagatctagtttgtcttaacataattctaattatggatgtaacgtaaattacaaatagttttcaaatgctctttttcccacaagtttatgtgtaaggaatatgtgaagttataacaccccaaaacaacattttttagttaatctgttttagaaatagtactgtgatttttatttcaaataaattatatattcagagtattgggaagattattctaagaaatcaaattactttgaaacattattactagaagtaaattggcttccaaaatctaatttgaaaatcacaacttgacacttaatatattttcctcagttttacacccagtcaggatgcttttggctcaccagtcccaaagaatattacttgtcagccccatgggaagctaacaagttgaccctttgccttgaagactgtctcctgcaaatgtggtgacctctccctttctgtataactctagtcactggccatcatggcacatgtaaattctctttctttattgctcaaaggaaatatgacatggacttcacacaataatatactaaagtctaaaaaatattaaaacagataaatctgtaaattaaagcatacatataattattgaaatcatagcctacttaagatttcatgtgatgtctaggacaaaattacaagtaaaataatagcggcaaaaggaatgatgagttttcagattatattacattctttaaatttcttcacactagatagattcccagattaaacatagagtattttcttcaacggttatgtatgaaacattttatacatcatattattattttcttcttagttggataatgactcaagaaactaagaaaggaactattattttatgacatcctaccaagtgccagacaatgatttcattctatcttgatacaaatcttatttaggtaggcattataaaacaggcttaatatgaaaagaagtagaatcaactggccactaatattttcttatattaattgttttagatactcctccataagccgtactttttctcctattttaaaattaacgtatgagaactaaaaaaaatgacaaactcattaaaggcagagattctcaatatcatttcctcagctgtgtgagctctgcttgaatagaagagagactcctggtattctcaaaattgagctctctttctaacgtgtgtgtgtgtgtgtgtgtgcatgtgtgggtgtgttagtcccttttgtgctgctataacagaacactacatactgggtaatttataaataacataaatttattctctcatagttctggaggctgggaagttcaagaccaatgcacgagaatttggtctaaagagaatcttcttgctctgaacacacatagtagaaggcagaagggcaagagagagaacaaagtctgtgtctccacatggcagaagagcagaggagacagaacctactcctgtaagtccattttataatgtcattaatccattcacagggagggagtcctcatgacagaaacacctcccaaaaggctcacttcccaacactgtggcattagggattaagtttccaacagaagaatgtgaagaaataagttcagaccacagcagtgtgtattgttttcaagtctatatgtatgtttgtgtatttgtgtagatacatgggtggctatatctctatagaaacaaaagtaatggtttttttgggggggcaacacattttggaaaatgaagatggtcttactagactaggatggttctgcagggcaaagaacatgtcttatttaattttgaattcacagtgtctaactgtctatcatggcatgaaacaccagtaaataatggatggttttgagtttatcataatgagaaatggataagagtaataacactttatagtggaggagaggtcaggaaacatataatactttttttaaaaagtactgtgaaactgtggttctacaattatttagtatgaaaacaaatagaattataaaaatagtggtttcagaatattttggtgccctcatttgtatttctaaagtactataaaatactcaagaaaaattaagcatatattatgtatattcagtttatcaaacaggaaatgcaatgtgcaagttaggaagtgcttttattgtaattctcaaaattgtatttttaaatcaagaaagaaatcttcacatttaaatccataagctttagttacattaaaaattcagtctctgcattatcatcctttcatttattcaaacacattttttattgagcacttactctgtgccaagccatgttcatggtgatgggggtgtatcagttaaaaaaaaaaaaaagtagaccaaaatctgtgccctcaagcaccttacattcttgaggtgagagatagaaactaaaattcaacaaatagtaagtgtatcagataatggagacatcatccagggagataaataaaccatggaagaaatacagagagttttgaaggaaaggggggctgagctttaagtaatctatgtgcttaagctccactgataaggtggtatttctcctgtgtccaaaaataagtgaaagcatggactgttcgtgtatgagcgacagtgccatagcagaaggaatagaaaagcaagggcctggggccatgataaagtttggtgtatttaaggggtaataagtactagaagcgagtgagcagaaacgaaacgagctctattggagttcaaggaggcagcagacaagataccagatcacttacaggatcttttaagccagtgcacaaatattagtttttattctgaatgagatggaaagccattggaggatttggagcattggagtgatagcatctgacttccattttctaagatcactctggcagatgtgataggaataaacagaagaaagcaaggtagagaaaagaaaaacaatggacagactatagaataaagaatctaagagatgatgtaaatgtggaccagacattgttgccaggtctggcattcatagggaatcgattttccctaaagttcatggtctttttcttttcatttttagatgggaagctatgttatagatagtcttgtctatcttttactcccctaaaataactaatttccctatttttttctgttcttcactgatgcagaatgctcacataaacctaaagcatgttcaggtcttcctcggctatatattcctacaagataaagtaatttatattttaccctgatttacaaaagagggtataaacttgtacttatgcaacatgaatatgttttatacctaatttacggcataaggactatagtcaacaatgttctattacactagaaagttgctaagacagcagatttggatgctctcatctctccctgtctctctccttctctctcacacacatacacatatgcacacacaaaagtaaccattttaggtgatgaatatgttgatttgcttaactgtggtaatcagttaactatatatacctatatcaaaacatcatgttgtatactttaaatatatacaattataaaaatgaaaaaaactatgctttttcctggtgcatttcaaatttagctagagggataatcaaagctcttcctcactcttcaaggattgacaaaaatgctatgggcagtgcaagaagtcacagcttgagcagtgcatcctataaagatatttgcataaagcatttaaattataacatttgaaaaacagtatgtgcattattcctcctagttgcctactgactggaaagcaacattgtagaatggaacgtcacaggctttgaaatcaaatcatgacccaaacccttctctgctgcttacaatttgtataaccttgggcaaagtacttaatttctttgagtctcagctttcttatctgcaaaatagaagttaacgtgtaggtcaaataaaataaaacctgttaaacaagtagcacattgttggtgtctaagtaaaggtagatattgatgtttactttgaaaatatttgtatatttaaatgacctttatattgttgtaaaaaaaagtttaaaaataaagctttcaatacttagttggctttgcaactttcaaaggcattttgtaaaatatccagattactattcacttgagtattcacaacatagaatcactgattaatcacaacattgattttttggtgagcttgtagttttgtaagtcaatgacaaaaattgaaaatcaatcttttacattattttgttctaggaatagtcagaattcatagatgtctgcatattagggatattctaaaatcatttcaagtaggagaactgtaataaaatatttgcttcagttgacaaatcacttaagttaattttatcctctcctgtgagttgggagaataatgagaagttaatgaataagtaatttagattacaaaaaaaatcttggcaatagtctgtaaggaacttaacagaaatgacatttcactaagtaaatctctgatagtgtgagtgaaacaataaattttactaggaaaactagaggttattttgtcagttatgaaacctgaccattaatatttattgaactgggtatatggaatttatggatattatctaccagatataatgcattataaagccatcattgtgggattttttgtagaaaaaaaatttattcaaatgacatgctagtcacatttctggttttgagtaagaatgccatatatacatatatgcatttatatatttcatttatacatagagaggaagagagagagagactggaaatttttttaaaaaattaatttgtttcaaagaaacaggctatcagtaagctaacttttctacacttatgttatttttatgactatcacagaatgctagaataatttgtattataaaatagacttaaaatttcctctgattttgtagattcctgcctgtggaaactaaaattggcacttatattcaacagaacaagcttaccagaagattcatttattatttctgatatactcttacagatatatacttttttttttttttgagacagagtttagctcttgttgcccaggctggagtgcagtggcacaatcttggctcagatatatacttttatacgagcattgcaaacccagactatagatttctggcactcaaaaatatgtcaagccatggaaagctaattgaaaccactgaatatttcaataaatcaatcagtcagtattgagaatcagctgtgtgctcagccctgtgcagtcaagtggacagggacatatcttggcagaagacagacacacacaaaagaaggattgtaaagtctaactaaccccacaaaaagatgttttaaacccttactcttataatatacatacagcttatgcatcagtttttcacaattatttctcatgcttgtctctcaaacttacaaaaatgaggtcagagaggcctagttagcaatcacacagttctctagcaatcacacagtcagccctacaggagtccatttttttaacaagagagaattttacaggctatagagagttataaaattttaattttcatatgcaacataataactcttaaagttgcttactttctcttataagattgtctttctctgacccagttctgagccccattttcctctgctctgggttgtgatctctaataaatctttagagtaaagctgttattacagtgtgaactgattgttctctattcaaatgatatcgtgtgtatacatgcacatggagtttgaagggattcaaattacagtgaataataaaaatcaataactttaaagtgaaaaaaatacaggttgaacatctgtaatatgaaaattccaaacccaaacagttctaacatctggaactttttgaatgatgacctgatgacataagtgaaaaatttagcacctgacttcatgtgacaggcagaagtcaaaactcagtcaaacctttttgtcatgctcaaaaaaacttgaatattatgtaaaattatcttcagactatgtgtataaagtcagtgattatgaaacatgaatgaatttcatgttgaaacttgggttccatccacaagatatttcatgatatatatgcaaatatttcacaaaaaaaaatgcaaaatctgaaaacatttatggtcccaagcattttggaatatgtatatttaacctgcagtttacatttacttatttttgacagacagtagtagttcaactcatgtgacacgtaagaattgataggcattaaaaaggtaaataatttaagtggaatcaacttatagcagacattgaactccacatatttgcaattttttcttgttattttatcattaccatggaggttatgggagttttaaggtgtcagtgacccacattacctttaatatgtcccctttcaataggatatcccaaaagaaaataaaaaggaattttaagaatgttaagatatagttgagcaatggtttcaatgatgtcaaaaagattcaggagaactactatacaaaaaatagagaatcagtaaggagaaatctgctgatggcctttaattaagttagtgatttaactaatgtaacatttggttagaacaagaccatctaatatagacagagttaaatttttaataataatttagactattaggaggaggccatcaaatatttgtcattaaattgttggagtctattcagtcatggaacaagtatttactgaacacttgctttaggcatataaccatgcactataaagcaagcttccactatccatacagagcttgcactttatttagtgagaaaaaccgtaaagaactaaacaataaaaatagctcaatgttttggtaagtaaccatttggtgaggtggaaaatgacaatgcactgacagttctctgctttaaataaaatctgcacatatacaccatggaatactatgcagccataaaaaatgatgagttcatgtcctttgtagggacatggatgaagctggaaaccatcattctcagcaaactatcacaaggacaaaaaaccaaacatcacatgttctcactcataggtgggaactgaacaatgagaacacatggacgcaggaaggggaacatcacaaaccggggcctgttgtggggtggggggaggcgggagggatagcatttggagatatacctaatgttaaatgacaagtttctgggtgcagcacaccaacatggcacatgtatacatatgtaactaacctgcacgttgtgcacatgtaacctaaaacttaaagtataataaaaaaaaaagagagaagccatccttgtcaacttgaggttagaactgatctaaaggagagaaaagataaaaaatagaataagagatagaaaagacaaagagaataaataaatggaatctgaaaaatacaaggaaatgatcaacataagagcaggaaacctcacaaattagacagggaatgactactgaaagccactcgggtagcaaagaggatgggatatcaaaggaacataaagatcactgctcatgatagtgtgggaggagagggaagagatagtgttagagagttttatttcatagcatagagtttgattttaattctatctccaatagaaactgagtgaagtctttcaaggagggaaagggtgtataatttaagattttaagaaaggtgacctgtattggattagagaaagtaagagtcaaaagaaagaaatgaatcatccggaaatattttttttctccacccagagactcaataatgtagccaagaatcattcagtaacaaggttcagaaataagaagctggagggtattaatgctttcattcatacagatgagctctcttaatttaggaaatgtagaactagaattgatatttcataaatagatatgatttacagcacaagaaattgtatattctcaagtactttaacatatattatcttatgttataaactgtgatgacttttaaaaggaaaatagcgatctttagaagtgagagtaaaggtaagatcttagagatgagaagcagcacaccatgggaaaagcaagaagaaggaggaatgttatgtgcaattaatcgggtcaggaaagagattggtataatgaatgaattgaaaggattttgtgggatgaagtaaatttgaaagacagcagtggcagttattaatatttttagattattaatctaaagatttaaaaattatataacctgtgcacattttttatttgtgtcttgcatttttagaattatgttctagatatcctgaaagcaccataccacactaatcccttcttgcacaaattgaggaaagatgttcataatccacaatgctttatcccaagaaagaacaatagattttaaacctatattgccaatataggagtgcaactgacatgtgtcccagaaattgtacctgggaaaactcaaaaattttactctaccaccattctagcctcaattcctctgaagattaactcaattctattgtcttttgttcatctagtattacatctttaacttacaatttaaacttcctaaatgcaaagttttctaacactaaagcattagtagtccattttgtcttacgatttaacttgagagttatacagctctaaataaaatgttattgtaaatacttataaatccttgaaaattaactgaataaaacatgcaatgtcattcattatctcctgtccttcttagagagagatctacatcaaaattatctcaaagagaagattcataaaacaaatgtgtacggctgagaaataaataaactgaaactctccatgagtcaaaatgacatttcttctaatgtttataaaagaaacataaatatttgtacatttttatgaaactatggtatgtatatttgaggattctttcaaaacagtttatgtaaaacgtaatagaattcaacaagtccaaagataagaaattgaaataaggaagggttcaaggaaatgactcaaagggagagatacaaagataggcatttgatcttggttaaatgagaggaatattataaaaatctaccaaaatctgaaaatgatacagatagatcttactctgaaagcaaccagacctggattttaattccagctctagaacttactaacttgtgtaaacttgaaataattccaaattctcttagagactttgttttctaatttgtaaaactgaaattataatacttcacaagcttttgtggaaaaataaattaaatatatattttatcataaggttcaacatatagaagttactgagcaaatattaatctaggtcttcctatatcctgtctctaaatcgaaattctccatgataaatacattattaagtaataagtaaattgcaaaactcttagattttgccagatactatagatagaaaatattactgaagtttaaatattgaaataatttactgatgcccataagttgtaactaattgaagttgatgtcaaggaataattgtgcatcttaacaataaactctttaatggcaattttagattattacattgattaactaatagtaaagtacagcaagatttatgttgtcaaaatctttggccatcatctctagtcactagagtgaatattgagagtaaatgtgaagaatgttcaaagttgtatttactattgaaggtattacctatttttagtgtgaacacccctatgttcctacgacatagcctaggtgtaaaggtcaccttgtgaaggcagtggacagttggtgaaggcttgtaccactgtaactctgctaaacttagaaaagatctgaaaatactactgataggtaagatcctatattctgtccacaaaaatcaaatgagaactatttgggtaagggaaaaaaatcccaaatatattatgttaagccaataacaattcaccattaagtaatgaaaaatacatatcaaaaagagaagtctaggaagaaaggagttatttgtacacagaaaattctcataataaatatttaacttagtgcctgaggttgtcaccaaggatctcactttttgaattattgtatttatttaagatcagaatatataaagaatatataacattggtgttatttgtaatttacagttcaatcgctaaaatgtcatttcagagtatgttaaatttaaggtgtaggctttttgaaaataagattttccttgatgaaatacaaacaggattgaatttattttgttaatttaatttgtacataatagatttagagacacgacattaaatctagtgaaaagcatttgttattaaatttcacatgccatttttctttatattatttccttagcatttaaagaaagtataaagaacatacagtcctgtattattttttgcctttgtgtattagcaagtaaccatcatttcccaagcaaatattggtgggcggtctcagctctttccttcccaaaacattaaataattaataatgtcacctgcttcacacttttgtgaaacctattctagatgctttatagcacttacatccttgtgggaaaagatacacaagcaaatatataagtaaataatgtcacgaggttgtgtgcagaaaaatgaagcaaccaaataaatagagaatgctgagtggagctgcaacagctacattagatactatggttagagaaagcctctctgataagatgacaatcaagaaaaagcctgaataaataggtaggagggtggacgggagtttagcagttaccaacaggcatgagggaagcaattgaaaagactttagccatgtgtttggtaccttagaaaaacaataaaagggagggaaagtaattaaatgaatgactcagttaactgtgaactctagtgatagaagatctcagtagcactcaatccccttgttttttgtttgtttggttgttgttattgttttttttccccggaaatctggttttccccaagcctcgccaatcaattcactatttttttcaaccactaattactgaagatatatcgttgtcatctcctttgtacactaatagtctagctttgaaatacagatatataattgcttgcatagccaatcttctatttaattacctttaaaggatttttaatttcttctttattctctttcatttaatgttattctacaatgtgtgtaatttgagggtttttttttttttggcttgcattcattctaatttaactcaatgggctgtgctgaagttcttagtggttgtgtttggtttcagctctttttaaaaaccgcatatattttactccctcctcttcgaatatcaattacacgaagttgtaaattatggaggtttcattcaccactgaatttttagggtatacctatcaattaccctaatttttaaaaatcttttctgtaactcattatctttgtatcctctgctatttctcaatataaactatatgcaaaatacagacaatggtattatgaggtcttatgtacccttcatccaaactttattttctttttatggacaatcttcttaaatttctacttctatccacatcacccaactacttgtcaatatattttgaagaaaatcccagaaggtacgtcattttagatacaaacatatttatgtgcatccaatgaataagagatcttttccttacgaggtaaacgccataccataatcagacaaaaattacaataatttcataacatcatcaagtattcaatgggtactcaaatttccctatctgatatttctccaggtttgtttgttcgaattgacactcacataggatatctacctattgagtttttgttgatgcccatattttaaattcctaaaaccactattttacacatgaactggaatacttgtttttccttgggtgagcttttgttttcttttaggtagtggtgagttttctgaaatgtttgatgatgtttggttgcctcctcatctttgttctgagaatttctgcttgcatctctatcaatcagttgcagatattgatgccagggatgtttcactgatgcacagtcagctggtctgtgctggcttctgaatgtcaggtctccacattcctcctagactttacatataaccgagaatacctgctctgcttctcttactcccaaaccccaccaccgggggttgtgggaggagggcagagggacaggggtacaaatatgcctgtttactttagctaaagacaagcggtgtgatgcaaaggagaaggggccaaatcatctggtgctactaacgcaggctcgcaattgccatcctgactattacaagaaggaccttcctggttttattttatctttagaatctgtgcttgtagcatgcttaggagtgtccccaattccgcaattatatatttgtacaagtgttaacatctgactttctctggccattgtaagctcatcatctttttactttttcaatatttccaaaagtttatttcctgtccatgatacactgcaagcaaagtaatagatatgtatacataaatacaaagatacaagtttcataaatagatacatggacagctaatgttccctcactttctcatggtacaaaaaaagctaaacatatatatttcctctaataccaaggaattcatggtaggatatgaggtgggtaaggtgcttaatctgactgatagattcaatacatggcctatttatttctaactataaattaggctaagttgtgctctctctttctccatacaggtagatgaatagatatagacatatgagtaaaccatatatctctctatacctatctatctgtgtatatattatatacataaatagatcgatgtaaactatttctatctatctagacatattatatatatatatatgaaatcttacatgggcaggtagatagatatatctataggcagatagctatgtctcggtagatagatgtattacagtttagatagatatgtacagtttacacatctataaatagacatctataaatagacataaatggatatctagatctacagatttctatacatatctagatctatatctacaaatagataaagataaccttatatctgtatatccgtatctatatatcaacagatacagagtttacacatttatagagttttctatagataactctgtatctgtaacctctgtatctgtatatctatatgtacctatctatatatacactatatatctatctatagatacatatatcaactataatatgttttggatttatatacagttttgtattatgaatcttaagctctgtcactctatctctctgtctctttctctatgtctttatctatttccatctcagagagacagagacagaaagaaagtttaaaatcaaaaactgtaaataaattcaaattggataatagctcaataatttatctcatttttatgtcacttaattctttctattatttggatatgcatcaatccttaaaatagctatgacctaatgagatggaaattgctcaaggtaggagatctagatcctattaactttgtgattttagtaagagtattgctagctgttctttaataatgaggttaaaactaaacttttaatatgaagatttctaagcataataaaatgttgttatccttgagaattagatatcatcgaaagaatattaagaattatgcacataattaattgaaataaattaacttagtgttctaaaaataaactaacatagagaggtatatatgtaaatgttaagatatatgaccatgatttctataatcatgtatttcataaaaatagaatttcaaagacattgagaatattgactttagaaaccagattgaagaaaaaagccatgatgctacctgcccacaacacgtactttcaccagatatctggaggcctgacatatgaaaacaaacatttttttttttctttgcctcatagagcatagtgaaaattttgtgggtgtagttagagagtacaaacatcaaatcattaatcatgttattaaaaagcaatactgtctaataaaaattaagcagtgcatttcagttttcagaattatccaagcagaggctaacttattcaagcagaggctaaacagatgacaatttattcacctgtttacttaattgtagatagaattcatgatttaagagaatgcgtgataaatgagtgagcgagttttcactaacactccttcctgctttaaggtactttcattttatgttgttatataatgaacaatgttgttacatatttattttactattttattaaattatatataattcatataattacatatgaattttactagggataggttagtatgaaaaacagtttatagaaggtttacattaattgaaaaataaccttttgtcatacttttgtttatttcgccttacaagcttaggcatttaaaacttgatttacctgaaagttgtttcttacattacagacatccactaatggctttattgcctgtctaatggtcattaagtgaaaatgggcatctagaataacacagctggggtccactttaccaagattctttttaacacagttggatatctacatgatgttcatacccactgttctttctgaacattagtgccccataactgtcttttacaatgtaattcattttctttcttcacttaactctgttgaaggaatcaaaaggctgaacccgtagttgtactaagaaggggagggcaaatggtcttgaagagtagaaacaccttactttagaggtagcattaagatgagcttctattgacttgagcttaaaaattaaaaaaaaaaaaattacaatctcagtagtgcatttcaaaggctcagaattattgattcagacataaacccatgtttaaaaatgctaacacatatgcagtgtaactagttttaccaactggtatactgtttcccataatgcaaaatttactcttgtgtttattttttttgtatactaaaatattgagattcttccccgcagtgtcctgaagccctcaatgttcgtatatctctttaaggtatcttatgtgtatgaaatatggctctaaaatgctagatttggccaacaacaacaacaacaaaatccccccacctttttttttttttgagacagagtttttcttttgctcttgttgcccacactggagtgcagtggcgcaatcccagctcactgcaacctccacctcccaggttcaagcgattctcctgcctcagcctcccaagtagctgggattacaggcatgcaccaccacgcccagctaattttttgtatttagtagagacagggtttcaccatgttggtcaggctggtcacaaactcctgacctcagacgatccacctgcctcggcctcccaaagtgctgggagtacaggcgtgagccaccatgccgggcccacaaaaatcaatttttagagctgttcagttcattatgtttagaagaacagataaattgtatacaacaataatatagctatatgttatagttattgtgtttttcaacaaaatagtttatttgaaggattggtatgttgttaacagatttcttaaatataatcatacttggattatgaactgtcagattaaaaataatattagtgcttgtatttttctgtgtgtgtgtgtgtgtgtgtgtgtgtgttatgttttcactaatcgaagaagcttgctggtatttctttcttggtccttggatacactcaggctttctttctaatacaactccctctatgcagatatactcaagatgagtcctccctatcttgtgtttaatacaaggtcatgtaatgcaatatatttagttaattatacctaacttttaaaaagtcctttacgtgaacctataattatatgtatttttatatgttactatgttctaaaggtgtttgatgtttaaagttcatttagaaatgttcttataagtagaattacttcaaccatctgtacagactagcaaaatattattctaagtgtctccttatgttttaacctgaactctctctctgctttaattgcaatagctatggcaaccaccccatcaatgacaaaaatcctagaaggatgtatgtataggaagttgaagtgttgagaagagaatggctcagagtcaagcgggaacaaggtaaaaacatcaaaaggttgtactggtgataatggccaggtgtgtgccattagtgcctccttgatattaaaaaaatatatggtggatcaactacatgttagtcaattccagcctagcaatcaagaggatattgaaagggaacttttcaacaagatagcaaataagttaagtgcatggaccatggactttctctccaatctacttgatatctaagcttcactaagtatccagatgccatcttgtgatgaaagaaaatgccattcttagtaagaggaaagacataattacaccacatatgttgcgatacactaaacacatttctttttttattacatgtaaatttaatttctcctaccttttccagctttgaatcattaaatcctgtgtaggtcagatgcagttaaaaacaaataggtctttactcaaccatgactttaaaattaaggattaatgtgtatatatggaaaaatgtttatctttaaattcacgttttgtagaaatgagcacacgcttactaataacaaaagttcatattaacatactaagaaggcttgaagaatataaaatacatttggagagactaaaatgtaaactttattgtgataagcctattgcaggaagaatgcgtgatacagatatgcataatgtgatgtaggggaagtattttaaaacaaaaagaaacaacttttgagataccaatggtgggtgtgagttgtgggagagatgggtaagtaatgcagagcaacaaaaatgtctaggaatgcacaagtaagcatgctggtttccatttcagattcaaacttcagagagagagggaagaaaaacatttaaatatatctggcataatccaagactatttacgacaagtgttctgtgtttctaataataaaacagacttcacctcggagtacctgcagaactgggaccccaatgaccagggagaatgaagaacaacttgttgaaggtatagagaaggcagcaaatcatacaagaaggtccatcccaaaccagagatagtatagataaatctctcatgttaactatggaaaaaataaaggaagatgacgtattttaatagaagggaaattgcgcttattgggagtaagaacactgaattgactgaatatcttaaagagagttaggaagtgaaattctcaacaggtgattattctttgaccaattttgtcttatctcttatccacattcttccctgctcttcccaatacatgtgtaagaatatccaagggcacacacataccataagcaggaatggtgtcctaaggaaagtcaatatagagagtaagaagtatatctgtcacaatgtgtacatgtatacatcctaaatatttgtacaattaatttacttctcttcatttccaacactcctagtctatgctatgaacatctcttacctaaaatgctacagatgtcccctaaatttttccataaatcagttttttcctctccaagctgtaagcagaaatgcatttttaaattgcatatatgaccgtgacacgtatctgtctcagtttatagaacaggtctctaacagggccagcagcttccagtcacttctgtcccttaccttttctccagtctcattttacatatttttttacatgctctctatattcctgtaattaaatattactttcccctcaatgcctttggactgcttctttcctccacattaaatactttttaccacttccctcagttcagttcattctcctccgtgtttagagctcagctcagatatccttttctcatggagacattctctggccaacccagactagatcattttcccttgctgtatactttcatacctccctctgtctttccttcataacactttggtaagtgtgtacaactgcatccttgtttttatttaatttcctctcactgccatgttctgtaaactcaatgcaagctgagtctgcctatttatttcactatgcgacacctaactcctaacaaagtattaaacaaaataggtgccaaaatatctgttaagccattgtgtgaatgtataaatgaataaagatatgaatacagatacactagtctaaacacgtttgcatggcacatacctatatgtgttccaaatagataacaaggagaatcacaattgatcattgccagaagaaaaatctggtaaaacttttactttccagatatttaaatagcattcatgttaaaatttttagttcacttttataatgcattgaaaatacttagggacattatttacttatcagaagtttattttactctttgaaaatatattgcttactggactattcacaatagcaaagacatggaatcaacctaagtgtccttcagtggggggactaaataaagaaaatgtggcatatatataccatggaatactacacagccataaaaaaacaaaattatgattttttttttttttttggcagcaacatagatgcagctggaggccattatcctaagtgaaataatgcaggaacagaaaaccaaatactgcttataagggagagctaaacattactcatagaaataaagatggcaacaatagacattggagactactagatggggaagggatggattaaaagggctgaaaaactaactcttgggtactacgctcacctcctgggttacagaatcattcttatcctaaaccttagcatacacaatatacccaggtaagaaacttgcacatgtacccccaaatctaaaataaaagttgaaaattaaaaatatatgcatattgctcattgagatagactacatattgagagattagctctatgaagataacatgaaccctaaattgtgtgattgagagaattctacacatttattggccttcattttagataacatgttaaataattcaataatgaacatttgactgtgcaacctcttcatataaatgtgcatattagtcaaggggctgtattattagcttaacaactaaagtctatttttttttttgcatgtgcaaagtccaatgtagaggtgtctggaagatctttcctaaatgtgactctgtcacctaagtggattgcattgtcatggaagccactatttcaagctgtagcctctaaaatctctacagccagggttgggactaatattcaacccttaaactatcattgcattgatcagtaatcaatcacctagttatgcctcacttcgatagagctaggagaggcagtttatggctgtatcagaaataggatgaaggtgcctgggcatggaggctcacgcctgtaatcctagcactttgtggggctgaggcagaaggattgcctgaagccaggatttcgagagcagccttggtaacatggtgagaccttgtgtctacaaaaagtaaaaataataaaatttaaaaaatacccagatgtgatggtgtgcgcctgtagtcctagctacttgagaagctgaagtgggaagatttcttgaacctaggagttcaaagctgcagtgagctatgaccatgccccttaaacagtgaaacagtgaaaaaaatagcataaaggaatatgtactgtaatatttaagtattggatttttagtaatgaaaagtttctaatggttaactgtgtttccccacaaagatacgtttaattcctaatgccatgtacctgtgaatgtaacaatatttggaaattaggtctttgcagatataattatttaagatgaggtcctaataatttgtggtaggtcccaattccatatgactgctattcttataagatgagaaaacagaaacacagaaaaaaggcttgaaaaaatacagacatagaaaggcagagactggagttgggctgctacagccaaggaatgcttggggctcccagccgttgggagagtcagggagatgctcccctacaggattagaaaatatatggtcctgctcacatattcattctggacttgtagcctccacaactacaactatgagacaacagatttatgttgttttaagccacccactttctggtactgtgttacggcagtcctctaagatgagaacaatatagaaagtcagtcatatccttccagaaattaacaagctggaatcacattttattcattggcctagtaaattcctttgccattctcagtatttctagaaaaatatcgggttgttgtgttttatttttattttttattttttgtctgataatgaaaaggataaaaaatcagctaaataacaggatttttgtcatttgaggatatcacttactaacaagagcttacatatttgaatggaccaacaggtaaataacaggtacctgtaggccatccgtattgtcaaactaagtgtaaattacaaaacatagaagacatacttaggattcaaacaataaagattcagagaagagcaattgcaatctctctcctatgcacagaaaactgagtgaagcattttatattttaatttgtgtttagtggtgatgacaagctcatcaaagaatggaacattgatgcctcagcattataggagacataatcttcatagccctaaatgctgcacaggcaggtacagaattgggcgcatgtctcatggtggccagcagcttgccttcatagaaaggatgctcgttgagcagaactccatgcactggttaccatgccagggaaggactccaactacatggaggatagcaaagctcatcccaaagccagccgacaggaagagaaatctaaacatgtctactcagtgcaacttcctatggcaattgccaaggaaaacatgatagaggcaagcactggattcaacaatgtgtttcctgaatagaaaagagacagagctagattagctacagtggcgtgcctcagtccaccatggctagtggctgcccttggaaatcaacacagggcaactggcttacatgtaccagaatagaaggactgcaatctgtcccctatgaagtctgaagtaccaaaatctgagggcatatctgagggccactgacacacatgcttggcctggataaaggaggagcacaccttgagtctgaaacgggaaaagatatccccacacgaggtgataagcccagcacaggctgtgtggactcttgttctcttgggatggaagtgtttcaggcccagtgcccattcctattcttatgtggctgagcagaaatcaaagactgcaaggacgagactacctagcccaacaatcataaacttcagttctgaacagaggctgcaagtccacccaatccacttttcctcaatttccttttctttgcctgactttttctttattatcattttgagtattccagaaacccattataagtcattacttcactgcagtcacaaggtccctaagggaagaacttgcccctccttttttgggcccctatgtgacacgtcagatctttagttcaggtttgtcaatactatttctccacttcttttataagctattaattcagtttccatgacaccaaactccttatcctatttcattgaccacatggttactgttctcattaattattgtgtcttttacttaatggctctctctctctctctctttaattttatcttctcactctacactcccttttttgataaactgtctattcctatggtatacttggccatcgtaattgtggtaatgtccagatttctatttctcatatcaccttcttccttgagtatggactcatgattctgcttcctactgggaagttctgacaacatctataatttacatgggtccatattgaactttttttcttacattcctttcccttaataacagcacttcttatctgcactactatgtacaaatttcatgataggaaatccaaggcagaattatcttggtgacactcatgggtgtaccactattaagattcttctgtcactcattttttgatcatgaatttatagtgactaaccagatatatagtacatgacataaaattgaatcaattatctaaaaaacaaaacaaaactaccagtggtaaatggatgattttcaaatatggcatttcaggctgttataagaaaacttttacacaaattaaattaatgatatttatctgagcaagaacaattgataaattaggccgcactctaaaacagaacagatgcagagagttctgctgaacagtgtgagcagtgggtttttagaggccaaacacagaagcaaattagaaaatcctctgattggctacaggtaggtgtctgacttatttgttggtgcaaagaggcatttcttttgcgggggcaagtctgatcagttgtttgcctgtgattggctgaagctcagttgtatgtaattggtagaaatctggctatttgttatgaaataatattctcctaagttagattttggtttatgtactaatttaggttgcagttcgttatatagcaactcaaagtacagggatagcctcagacattaactgaggtctcctattttttaatttaataatacaagaacagtcttccaagatccaaatacacattttaaaattctgtacatctataacttcaccacatcatatccaaattgtatcctacttttttgccttttatattaatatttataataaaatttgtataatattaagtataattatttcatttaatagtattcacttaatggtactcatgccactcatttgcccaagctaaatcttacagtctttccagttcttacaattttaaaaatctcctcgtattcaggaaatcatcaagaccgctagcaggagaactcccttacagacctagacctcccacctctagactcatacatgagagagagagaaagttttgcttttctcaagacacagttcttttgagtgcttcttttactttcagccaaacttagttccatttaataggttttaatatatacttaaacaaattattatagtaaaagtaatgaagtaaaattatttcaacttacaacaatataaaataaacgtttcgtagggaaatgaacagaactacttaaaacttcagaacacaaggctattttactagattttgttcagtttaatagaagcgtgttccacatcacagacaagattccagtgtttagttgcgatactggccagaaggaggatcccttctgagatgtgatcactgtcaagaaagacttaacacatgagagagatctgcaattccattgccaatcattcaaagttccttgaagatcaacaatttcatcgagaaagacttgttctttgaaatttagaatatccagaacttttcttacataatagcctataaacttcagtataaaacaataatcattaatagtcactggtttatttctttaaaaatcttgtcaaacaattttaaaattcagcggaatcgctccttaatgcaattaagatgtttagcagctttaagtagttatcttaacatttctgtatctccagggtcaaagtgtcatattcatggctagattttgtagcaaaagcagcagtttatctatgtgaaacacctcttattggttcagcttatctcaatatgttatttctatgccatggtgatatttgctatgaaagcactgatatattttagaagatattctccattcatatgggtccttctagtttggcagagctaaatggacttcgttatttatatttcatctgacaaaatggaaaagtttaggtgaactgacatgaccagattttgtttgtagtacactattttgatttgaattattatagataatttacatttctaaatgcatctctaattccaaaatgaagaactactgtacttcagcaatgatactgtttttctggctccagatcattttaaagcaggtttaataaggaccctgagaattatctcaactgtgataactgctgtgatatttgcaataaaatgttataaaataactacttaatacttactgtattagtcaattcttacatggctataaagaacaacctgagactggataatttatgaaaaaaagaggtttaattgactcacagttccatggtctgtacaggaagcatggctggtaagcctcaggaaacttacaaacaagacagaaagtgaaggggaagaaagcacattttaccatggtggagcaggggatagagagagcaaagggggaagtgctacacacttttaaatgaccagaccttgagagaacttattatcacgagaacaacatggcggaaatccgcccacgtgatccaatcactttccaccaaatccctcctccaacattggggcttacaattcaacatgagatttgggtgccgacacagagccagaccatatcgcttgcaatgtttccccctgttgtgctacattttgtttatcaggactattgcaattttagacttggctttaacaatacattaaatttaattcagttacttgtaccttcaagagatttttcaggctggatagctctgatcctgaagatgagaccagatttaaattaaaaaaaaaaaaagttaactttcattaccaagctgcgaaaatctaaattttaaatgttaattttttatcaaatttgttaattttatttcatttaaagctgaaaattttaaaatgcaaaaaataggttctattgtcattgtgttttgatctgggttttataaacctatctcattatatttattcattcagaaaatatttattgagtgtctcagttctaggtgctatagatttattaataaactcaacagacacaaatatttggcagttttgttcacagtcaccaataatattcatcatttcaatatttagctgttaaaaaaattaatcactaatatagtatgcattccaaaatgaaagaggagagaggaagggaacataactaaataccaatctatactttctatatttatacaaattccatacactaaacaattattagtaattagaggtaacattttaacctttaatttaaaataatgtaaaaattcataatgaagtctagtacacgataattctcattaataatttaaataatttataccaagtagttatagtggttgattcaatgaaagacttattggaattcatgcctataatttataagatctgccaccctaccagccttactgtttttctcattggtaatattcatgaagtcactggtaattttacattttaaaatatgcagtatgaattgcatatatagtacttcttaaatgtcaacacatttatcttaaatcatttatcgaagtatgagaagtacctatcatattttggtaaataatacctttaggtttttcctagttcttggctccagactaaccatcttgacctgtcattctagtttttacttctgagacattctatagtctgtgtctgatattctctactatttcctcatttgtccttgcattcagattgccttttctgactcccagcttccacggagagattaactctgttggctgaagccctattcccaattccttggctagaccctgggtccttcatgttagaaaacctggctttactactactactactactactactactactactgctgctgctgctgctgctgctgctgctgctgctgctgctgctgcattttttaaaaatatattatcttattttactatttgatgttataattgttatatatttttccacacttcctcatactgcttatctcttacttaagaatttatgaataaagaattgatttttcaatacatccttccaaaaattatctgatgttgagttagttgctctctcttgtgcattctcagtcctcacaagcctttctcaaacacaatgtttatcaaagaaaattgtagcaaccaatatacttagtggaatttctcacagagtttgagtgtaggaaacagtattcactgtatattagtcattttgctcccaatagaaggtgc

SEQ ID NO: 12 - DNA, 95264 bp, Homo sapiens
gtctccagcgggagcgcgagacgctggtcaggctccgcggcgcagctcgaaaaggaataatcgcccccgattgactgaaattcctccggagccggcgccgcggccgcccgcgcccgagaccgcgctccggggccgcgtcctcctctcctccggaaaacgctcgcgacccagggccgccggcggccgcgactctgctgtgtcgatcgcctgagtccgttttcaccgtttgcgggatctggaaccgagttacatgcatgtccagtgggggcaggtttaattttgacgacggagggtcctactgtggaggctgggaggacggcaaggcgcacggccatggcgtctgcaccggccccaagggccaaggcgaatacaccggctcgtggagccacggcttcgaggtgctgggcgtctacacctggcccagcggcaacacgtaccagggcacctgggcgcagggcaagcgccacggcatcggcctggagagcaaggggaagtgggtgtacaagggcgagtggacgcacggattcaaggggcgctacggggtgcgggagtgcgcgggcaacggggccaaatacgaagggacctggagcaacgggctgcaggacggctacgggaccgagacctactcggacggaggtaggtgccgcgggccgggccgggccggggcgggagggacgtgcttccgatcgcgccccttctgtggatctctggggaagttgagctgcgtcctccggttgggccgtgggcaccaggggcctttcccgggcttccctggaagccacggcggctcctggctacaggttgccccgctgcctggtggggacagtgcccctgtgccagctgaagggtgttgggggctttccaggggcagagccaggcggggcccagcagagcctccgtgcgtcggacacagcaggccggagacctgcgggaagggccaggctgggcccgcggccctggggaacccagcaagggcagagggggctgggagaagggcccctccccttgttggctttgccccccaccctggaggccgcccttcgatcaggccccagcttcgcttctggtgttcctgcggcgcccgagggccttcctcagccccgaatctgcccaagccgagagaaagggagtgatggggagcgctaggggcgggggatgccaacccgaaccaaaacagccaggcagtacacttctaggtggttggacgcggactagcgctgtcgaagcaggctgctggagggaggagggaggcccatctgctcagtgagagcccaggaatctcgtctttcagtggctgcatcgttttcaccattagttgagggaatcgatctgtgccttcattctaagatgccaccgcattcggggcagagccggggccggaagccagggagctgcctgctgctgctgctgctgctgctgctgctgctgctgctgctgtaagatggtttctgtgcagggaaccttggccggctctgcagctgcccgcctgcctggactctccgatatccactcctcagtgcacctgacacgcatggagccggtcctttcctggaagccagaccccaaacaaactggcttccccgaccagtccactcccatgtgggagcttatcctcagaggcactgggtcctctgcctccctcgggcggctcgcctgtttcaggcatggatgcctgggaagggagtgagacgcagcaatgacttgtgcttctgccgagaataaaaatcctgagcgtacgtgtgctctagtcctccccgcccgctcgctgcctccgagtctcatgaagaaagctcggaacacctgctccacggccacatccaggagtggcagggggctggagagtaattattattttggcttatgcaactgaaaagcagggttcgttattacccgaagaggaggaggagtctggggcagttaattatataatgctcctgcctgtcaaagccgtgcccagccccggtctcaggcggccttttctgtgccgttgttgttttcatggagcaggttggagagcttccctgaccctggggcgctgagggccgggcgggagcagacggtgctcccttggttccaggctgtgggcctgtgtgtgtgtgtgcgcgcgcgtgcaggcacagggaggcccgggcagcagatgtgggtggctgagacaggcaggcagcctggcgcttccagctgggctgtttggagcccaggggctgctatcacccccaggctctggccaggaggctctgcagacctgtcctccagaggtgctcctggctgagcctgctccaggaggactgtgcaggccgggctcacgcagggcatggggacgagaggagccagggctttcgttgtctctgaaactcctcagaatatctgggggtgaggctgagcaccccactctgcccacgctcctcagacatctccagagtcctgaggaatcatttctttcattcaacaaatactgatggaatgtttgctatgagccaggtgtgccaggctctgggaaaaccacactgagcaaagcagacagccaggtgtcctaggctttggaaaaaccacactgagcaaagcagacacagatctttgccctcacggagcttacatttgagggagaaggagcggcgattatgttgtgactttttagggaagaaaggatctgattctgtggctggccgtgtagtttaggatctgacgggttaaggggagagatgtttttctctataggtccacttccctgggctctccactccagccagacagctccctcagcatcctgctcccctaagcccttcccctccttgtggtcctgcctgatcatgtccattctacagatgaggccagtaaggcccgggctttgtgattcaggagtcccagcctttggctgcagggatccccgatctgtctgggaccaaacccccctcgttcttgcatcacattgccggaagaagcccagattgtcaacatcctctcaaggggtatgcggttcttagctctcccataagcaaagccaggcagggatgcagatggggagcttcatggccggctctcccttttggttggcaacatggcatgacaggaaatagaattgacccttctggtcttgaggactcgggcctggacatctgggccacttttctggagggctcccagcttcctgttgggaagggggtgattatggcactgagtgtccccaggcagcccatggaaggagcctgcagggtgtggggatcgccagaatggggcagagacctccatgcctccttcccagtcaactggtgggtggtgacagccatgtcgcagtgaggaaggagttaatggccccagctccactctgacttttcctttgcacaagaagtgggggaatattgggcagccatcagcgaagctctggaacctcagcccagctctgggcgctgacttggggaaatggcgattttcatgaatgatgcaccagcggccagcgtattccacatgtagctggaattggtgtggaatttcagctggggacacctgtgccaactcctcctcctccaggtgtgtcctccccttagggtactccctgattgagctgagtagctcctaccgtctgcatggtgaggcctgggggtgtggggtacaggcacacttgcttccagtcacaaccccagcttgcatcctgggcttgaatccctgccctacatgccctgttttgtgaccctggttggattacttaacctctctgagcttgtttcttcatctttacaaaaggttgataacaatacctgtaaggtgggtgaggattaattgagaatggtgtgcgtggctatgccaggaatatagtaagcatacattattatttaccgttagttttttcattacagtttccaggcaatgcatgtcacttacaatgatcattattagaaccaacatctggacaggagactggtgatcatgtaaatgatgttaaatagcaacattagatacttactgtctgccaagcatgtggcaagtgttaaatgaattacagcccttaattcttacacctctagagggtgattttcttgttattctcattttccacactgggaagtggaggctcccagtgggtgtgggttgggaggaaccagcaggcgtcacactcaggacagccatcccagagcctgggcctttgctaggtacgtcgctctctaaatgcccctgtgcaaaaacacctcctgttctttcctctcgtttatatctctcgttctcttgtgcgtacagtggcccagaattgcaggtgaatgcccatgcacagacgggcaaacccatacactgcccaggacccctggccccctgaggcccaccatgcatgccggtgctgcaccgcacaggtatagatctttccttcgggtgctgtgtacacagaacccctcgaatcgaaagttaccccctccaataagccctcctacaccctgaaagatatgagtcatctcacgcccaagcctctccccctcatactcaggtcccgttcaccggcccagattcctcaacccacacaaatcacacacacccacagtcggaattcagatccccccaggcgcctgcgcacacacacgcgcacaaaacacctgccacccggagacattttccaccccgtcccccagaacgcagccactgcgcccttgcggccttgcttgctctccactggccaaggactctgaatctccaccaggcctgtaaacggggagctcagatgacagaggcagtggtgtgcttcgtttccattttaaacacacgtttatgctgttagtgcccttttagggagaaaaatccctcatctcaacaaaataaactaccatggggcgtatgctttgtgcttggctatgactatgactatgcaacaagtaatttctttttcaaatgagtgcccaggttagaaggaatcaggttcattggtgcggagattatgccaacagaaactcatttcagaaacaagaagcaagccctgagcagttggagacgaaagcgttttctccgagattctctttccgctacttcggggggcgggtggtgaaaggctccgtaaggtgaaaagagaaaggaagaagcaaacagctatttttttgttgccagttatagctgagaaacaaatgtggctgaagggcacccctacccacgttggccgtgaggccgtttgatgggctttgtgtgaggaatcctgagatgagctggccggggagtgaggaccggcacccgcctacagcagcgatctggagtagtgagtgagcagccccagctgacacagagaaggagagagagaactctgaccccttctcttctgaggacacccagccggagagagcaagaaggggtggagtgtgggggtgggggcaggagcctctgctgttcctttccaagactttttcttcctgttttccccaagcctccacaactctaaatatgacgagctctcaactgtgagctaataatgttgaatgtaacgccctttgtgtccttagatgacaaaaagacttgtgagtactagaggagagggtgatgttggtgtgaattttccacattggtccgtgtctccgtggactggccgcttcccccgctggtagcttcatggctgtgagaggccatgcggtggatggagccaccgctgcactggggtcctggctcgaacttgagtaaggtgctgtcttccaagtctggagaggagaggaggagagaagagaggcagtcccttcagcaccttggagagcgctcccggctgcctgcctgggttgggtggacggctggactcactttcgctctaggcatcctcttcaagccaaaccatctctccttgccagtgaccgcctggtcccggaccctccagcacactcaaccaccagaagtgtgcctatgcctgtgccagatgtgggggccacgcctgcgatcccaacactttgggaggccgagatgggaggttcacttgagcccaggagtttgagaccagcctgggcaacattgtgagacccccgtctctacaaaaaatacaaaaattagccaggtgtggtgctgcacgcctgtggtcccaccactcaggaggctgaggtgagaggatcgcttgagccccggaggtcaaggctgcagtgagccaagatggtgccactgcactccagcctgctgacagagccagaccccatctcaaaagaaaaagaagaagtgtgcctggtcctactcagtcggtgcatggattccatgctccaggaagggctgccacttggcatgcctgcctatgttttcacccctaccctgtcagtccttcctgaagaagtgagtgtcttctgaggtaacatcctctgctggtcaactgggagagcagctcccagcttgtggaggtgggtttgggggtgagactttatttttttgtttttatttatttatttatctgttttgagatggagtctcaccctgtagcccaaggtggagtgcagtggagtgatcttggctcattgcaacctctgcctcctgggctcaagtcattctcatgcctcagcctcccgagtagctgggattacaggcatacaccaccacacctggctaatgttttgggttttagtagagacagtgttttgccatgttggccaggctggtcttgaactgaactcaggcgatctgcgtgcctcggcctcccaaagtgttgggattacaggtgtgagccactgtgcccggcctagggtgagactttagtaagatgtatagaagttgctctgaagtctaaggtgtattcctgccataggatatgtgtgttcacttctcttggagcataacaggggtcataggtgcacagccgaaggcaggcagaggaatgatttgtctgtaccagtcagtgaaactgtttcaccaccattctctttacatctgcctcaatgggtgagtcctctagaccaatggtccccaacctttttggcaccagggacctgtttcatggaagacaatttttctaccgcccagggttgagggatggttttgggatgattcaggcgcattctatttattgtgtgctttatttctattattattacattgtaatatatagtgaagtaataatacaactcaccatcatgtagaatcagtgggagccctgagtttgttttcctacaactagacggtctcatctgggggtgatgggagatagtgacagatcatcggacattagattatcataaggagtgtgcaaactaggtccctggcatgagcagtttccagtaggctctgcattcctatgagaatctaatgctgctgctgatctcacaggaggcagagctcaggcagtaatgcaagcgttggagagcggctgtaaatacagacgaagcttcacttgcttgcttactcactcgccactcacctcctgctgtgtggctgggttcctaacaggcaacgaaccagtaccagccacatttcacgggctcaacagccacctgggactagtcgccaccatattggacaatgtaaatccagaacattccatcagtgcagaaagctttattgggcaggcagcactggtctggagtgtaagttccatatgggaagcggtttgtgtccatgctggtcactgccatgtgcccagtgactaaaatggtgctggcacgtggtggtcaataaatatttgtggaatgaatgacctgtgcaagtaaatgcggtagtatttggtaattttgtttaccgtcattatttcgtatgcctgtttcattgcaagaccttagaattggacgtagttaaaggaaagtttttccgtgagaccctcatgtccacaggtcagtctccctttacaattagcattaaaacatgcctgttctaattccttgatcagttttgttttgtggttggctttgtcattgctccttaagagactgccagtgtttcatatgagaaagatgatggtgttgaatgcagtgaataatggagcgataccagcagtcatgactgggaggcatttttggaccacgatttaatgagtagcattcattgcaaagtgcaaagaaaggcccatggaaagatgagtaccctccagtctgcttaattcctcactatctttttactgctcccttttccttggcttgattcgttcagtcattggagaagcatttgccaagttcagaccgtgttcaaggcacaaagacagaaaagctcaggtccaacctcagtgaatcagccttaagtatttgctgagcattgagtatgtacgcagtacagagtttgtggtaacgtggaagattggaaaaaaagaaacaaacacataaaaatgaaaatgcaagaaataggatgtcctatgttctaccaggaagaataaggagtctgaacagggatgcaaaaccgagatatatggaataattcagggtttctcatgccacagtgggggtcctgaatgggagcaccacagggcagggacctgaggaggggcagggcaggtgtgtgtgtccctgaaggagcggcagaggcttctgggggtaggcagtgcctgagttgagtctggaagggcagtagggtgagagttgttatcccaagcctgaggctgcagatttgttcgaggccatgtctccctcagcctcagcttccccatctctatgatttagtcatgacatttttttccttctcaacatgtgccatctccctcctacctatgaatgtcacgggtattgttctggacccagggaaggctcgctggtgcaaatgctttccaaaaacaccgagagtcgctctctctgctgctggtggagtctgagttgggcaggacatgggcccgcctctgtctgctgctctatgccagtgcttcccaagcctgagtgttacaagaaccacctggaaggtgtgtcaagacacagatttccagccccacccctgagtctctgggcctgggatgccacccaagaatttatatttcttacaaatttcaggcgatgctgaggctgctgatctggggacctcacattaagaaccacagtcctgggcttgtgcctcctaagcctggctacacattcgagtcactaagaatgtccctgggagacggttagaaatgcagcgtgtcaggcctgcctgacctccagggtcaggatgtgtgtttggagatgctcaggtggctgcagtgcaggagatcctgctttggtgcactcactttaaccctagctgtgcatgagcttcatctaggttgctttagaaaatgcagatgcccgggccccacccacaggtgctgattgaggtcctctgggtacagcctgggcattgggattctttttattttaatttttttcagacgggggctggctccgtctgtcgtcctggcgtgcaatagtgtgtgatcgtggctcactgcaacctctgcctcctggcctcaagccatcttcccacctcagcctcctgagtagctgggactgcaggcatgcacccccatgcccagctaagttttgttatttgttgtagagatggggttttgtcgtgtttcccagactggtctcaaactcctgagctcaagcgatccgcccaccttggcctcctgaagtgcttttacaggtgtgagcaacttttttacaggattacaggtgtgagcaactgcacctggcctgagattattttttaaacattccaggtgattctaacgttcagcgcaggttgaggaccactcatctggagcgtctgtagctggacatcacttgggagctttgatgactgacccatgccggggtccatccaagaccactgacactggaaactttaaggcgggggtctcagcctgagtatctggtcacagcaagcagggcctagagttgcagctgtagataagagtttttcacactctgggtcaagtggttcatgaaattaatatagtacattaaaatagactagaggccgggcgtggtggctcatgcctgtaatcccagcactttgggaggccgaggcgggcggaggccgaggaggtcaggagattgagaccatcctggctaacacagtgaaaccccgtctctactaaaaatacaaaaaaaatattagctgggcttggtggcgggcgggcacctgtagtcccagctactctggaggctgaggcaggagaatggcatgaacctgggaggcggagcttgcagtgagccaagatagcgcccctgcagtccggcctgggtgaaagagtgagactccgtctcaaaaaaaataaagtaaaataaagtagactagaaattttccaagcacaatacctacgtttctttctttctctttttttttttttttttttgagacagagtctcactctgtcgcctaggctggagtgcagtggcgcgatctcggctcactgcaagctccgcctgccggattcacgccattctcctgcctcagcctcccgagtagctgggactacaggtacccaccaccacgtccggctaattttttgtattttgtttagtagagacggggtttcaccgtgttagccaggatggtctcgatctcctgacctcgtgaaccgcccgcctcggcctcccaaagtgctggaattacaggtagcgtgaactcagctcactgcagcctctgcttcccgagttcaagccattctcctgcctctgcctcccgagtagctgggattacaggtgcccacgaccatgcctggctaatttttgtatttttggtagagacggggtttcaccatgttggccagactggtcttgaactcttgacctcaagtgatccgcccacctcagcctctcaaagtgctgggattacaggtgtgagccacctagcccagcctattgtgttgtttttgaatatatttttgatctgcagttggttgaatttgtggacttggaacctgaagctatagagggctgactgtgtatgataacctagaactgcaaagccagggaattcctttgaaagaaaaacactttttctttctaaaacatctattttgttccaagtattgtgctgggcattttatcatagacgttatatcttacattccttaagataatgccttgagataaggcattccgctcccatttaaaagacaggaaaggtgaggctcacagggtcacactcgctgagatcacagcgctgcaaacttgtttctggccgcagaattgtttccggcgctccgagatgcctttcttgatggagccagcattccaggggcccacttgctttacagatgaggaaactgaggcccaccaagggaggggccctgccccaggtctcacagtgaagcaggggcagagaggtttcactcccagcaatttgtaagcttgggggactgccagcgtggtgtggacacagagctgcgtgacccctcccaggataaaagcaggtccctgcagatacgaggagccgcgtgtcccctcccaggataaaggcaggtccctgcacacaggagctacacgtcccctcccaggataaacacaggtccctgtacacaggaggagctgtgcgtcccctccaaggataaaggcaggtccctgtacacaggaggagctgtgcgtcccctccaaggataaaggcaggtccctgtacacaggaggagctgtgcgtcccctccaaggataaaggcaggtccctgttcacaggaggagctgtgcgtcccctcccaggataagtgctggtccctgcacacacgagggtccgcgcgtcccccgcaggataaacgcaggtccatgcacacaggaggagctgcgtgtcctttcccaggataaaggcaggtctctgtacacgtgtggagctgtctgttccctcccaggataaacgctggtccctgcacacaagggggtctgcgcctccccgtcccaggataaaggcaggtccctgcacacgtgaggagccgcgcgtcccctcccgggataaactctggtccctgcacacacgaggagccgcgcgtcccctcccgggataaacgctggtccctgcacacacgaggagccgcgtgtcctctcccaggataaacgctggtccctgcacacgtgaggagctgtgcgtcccttcccgggataaacgctggtccctgcacacacgaggagccgcgcgtcccctcccgggataaacgctggtccctgcacacacgaggagccgcgcgtcccctcccgggataaacgctggtccctgcacacacgaggagccgcgcgtcccctcccgggataaacgctggtccctgcacacacgaggagccgcgcgtcccctcccgggataaacgctggtccctgcacacgtgaggagctgtgcgtcccttcccagaataaaggaaggtccctgcacacatgagggttggtggctctcctgagggttcacaggttcccggggacccatccacagatccccagataagaacctgtccatcctccccggggcctaagcgtgcccagattgatctcagtttgcccagtgagtagaaaaataggccgggatcagcagattcctatagacctggtggaggcctgggctggagtagacccaggcagagttggtctttcacctcgcccccttccggaggccccgtacccttggggtcagcatccaggcaacatgggatacccagaaccttctttcttgtcctgagcccttctctcccagctcggcatggacttgagtgacagctgctgtgtttctttcctgcccagtgagtggggccgggccctggagctttccctgttcatttgtttcgtctccttgtggggaaagcatggctcccccattttccagatgggaagcaggctcagagatgcaacagaagtgggaagtgatggagcagggactccatagctccagtcacgagtctcaaactctgtgcgcatctgggtcgcctgacccggcctccctgacccgggctgcccagaagggagcccaggaatgccggggagggtcacacagccagggccattcaatgcaggtggtgctgggaccacgccttgagaagctccagtcgccttccctcctctgcaatgaggcattgctagggggaggcgagtgggggaagagaccgggtctgttggggtcagccccgggcacagcacccgacagcagaggagacctccggaaatcatggctggatgggtcaatgagtggtcaatcaggcgagctctgagcgacaggaggggccggtggtgtggagagagggaagaagagcctgcaggagggagacggtgcgtgcaggggcaccacggccagagcctgcttggtgttcagagagccctgtggctgccgcagttgggagaagggcacggggtctgagcacagacagcttccaggccccagggttgggggactttgaattttctccagggcccgggaacgacattgtctgatttaagcagaacagcaacgcaatctggttttgtgtgtgtgtgtgtgtgtgtgtgtgtgtgtgtgtgtgtgtgtgtgtatttttttttttttgagacagagtcttgttctattgcccaggcgggagagcagtggcgcgatctcagctcactacaggctccacctcctgggttcacgccgttctcctgcctcagcctcccgagtagctgggactacaggcgcccgccaccacgcctggttaagtttttgtattttttttttttttgtatttttagtaaagacagggtttcaccgtgttagccaggatggtcttgatctcctgacctcgtgatccgcccacctcggccttccaaagtgctgggattacaggtgtgagccactgtgcccagccaggttttgtatttttgtaattacactttttattttgagatcaccgtagattcccaggcagtggtgaggaatagatcagagatccggtgtccccctcatgtggtctctcccgaggtggcatcctgtgcaactgatgacatgtcggaaccggatattgacgtggacacagtcaggatggggagcacccgcgccccggggtcccttgtgttgcccttttgtagccatacccgctgccctggcagcctctgagctgttctccattccttcagtttcgtcgtttccaaaatgctctgtaaacggaaaagtacagttcgtcattttggggctggctttcgtcactcagcctcatttgctggagattcttaggtgttgcctatatcaatagttcgttccaggctgggctccgtggctcacgcctgtaatcccagcactttgagaggcctaggcgggtgggtcacctgaggtcaggagttcgagaccagcctggccaacatggtgaaaccccgtctttactaaaaatgtaaaattagccgggtgcggtggtgcgtacctgtaatcacagctactcggaggctgaggcaggagaatcacttgagcctgggaggcggaggttgcagtgagccgagattgcgccgttgcactccagcctgggtgacagagtgagactctgtctcaaaaaacaaacaaacaacaaaccaaaaaaccaaataaaatagtcctttctttcttattgctgggcggtgtcccacggtgtggatgggccgtttgtttaaccatctgtcccttgaagggcatctggggtgtctccagcgtgggatgtcgtgaatttaaaaagctgctgccaactctcaggtccaggttttcctgtgcacagatgtcttcgctttcccccagtggatgccaggagagctggtgctgggccgtgtggaagttgtatggctgagttcacgtggtcactctggtgctggcagggcagggaaatggggggccagagaggaagctgggggtcatgggagggatgccacctctagggaggggtgcctgtgtccaaggagtggagatagtggctgggtctggtgggggctgtggaggtaggggggctgcatggtagggcagaggccaggagtctgggactgggtggtcctgtctttgagggaatttggagaaggagcaggtgcaggtgcaggagctgagagtcacctgtgaaacatgctcagcagagaagctgtgcgcagccggggcaggggtgcgtccatggtggggatggtgtgtctgggtggaaggggcggggcatctcccactcccggtggcattgaatagtgcgaggggagtggctgggcgcggtggctcatgcctgtaatcccagcactttgggagactgaggtgggcggatcacttgagttcaggagtttgagaccagcctgggcatcataacgaagccccgtctctacagaaaatacaaaaattagctggggcatggcagcctgcgcctgcagtcccagccactccggaggctgaggtgggagaatcacttcagcccgggaggtgcaggttgcagtgagttgagattgggccactgcactctagcctgggcgacataatgagacgctgtctcaaaaaaataaataaataaataataaaacttggaaataaggcaaaaaatattatttaaagaaaacagaaagtgggaggggagaaatgcacaggatggagcctaggggagccccaggactcgagaagtgggcagaggaagaggaggcaggaggagggagggaggccgggcagcagctgggggaggagggcgtggacaggccgggaaaaggagcaaaggcaggatgaggacacaacactggcctgtgcctggccttgtggcaaacacctcatcccttaggccattggccatcgtccctgctgtcctacagccccgagaggccaggtacgtccatgtgtcgtgcccattttacagacggtggagctcaggcctgggagacagactgacagtgttcggcactaactccaccccagcctttttccacctggccacactgtttactcgttcccgccagcagcatgggggctctggtgtttctgcgtcctcatccgcacctgtcatcgcctgtctttttggttgcagccacttctgggatggatttcctggaggctggtgctgagtctctctaggtgcttgtcggcccattggtacatctttggagcaatgcctgttcagatccttgcccatttaaaaattgggttatttcaggccgggcgcagtgctcacacctgtaattccagcactttgggaggccgaggtgggcagatcacctgaggccaggagtttgagaccagcctggccaacatggcaaaactgcatctctactaaaaatgggaaaatgagctgggcgtgatggcaggcgcctgtaatcccagctactcggaagctgaggcaggagaatcactcgaacctgggaggcagaggttgcagtgagccaggattgcaccactgcactctagcctgggcaacagagtaagactctgtctcaaaaaaaaaaaaaagggggggcactgcgcaccagcctgggcaacatggcaaaaccccatttctataaaaaatgcaaaaaattagctgggcatggtggtgcctgtagtcccagctatgaggaaggctgaagcaggagtatcacttgagctcaggaaatcaaggctgccgtgagctatgatcacgccactgcattccagcctgggtgacagagcgagaccctgtcttaagaaaaaaaaaaaaaagaaggattatttgtcttcttactgttgagttgtgacattttcgtaggctgtagacaccactgtgagccaccggctcccctgcaccccagcccacctggctgcaggctgcctgctccgttggaggttggacttctgagataaactgcgctgggtgtctgtctcttggctccagagtaccaggcacagtgccatgcacacagtaggtgctcagtaagcggcgacaggacaaagccaggatctcagtctgcggcttggaggggtctggggtccggctgtcactatctggctctgcttaccagctccggtcctcggacaccacgacggctgcaggcagagggaggagccacggagtctggggggatgttgggccacacaagtatctactgagcgccgactgtgtgctaggcaacaagctggccctggggacagtggtgaacaaagagccctaggacttcttggttgggggaataggacccgtcagtcagccctgcacagcttcctgcggggcacggatggggcgcaaggaaggggctggagatgccttctagggagtgaggctggtgagcgggttggggaggaaccatggggcctcccagacttcaggaggcgcatctgtgagcaccagggacagaaggaacagggccaccctgggacccagtgcgcaaggagcccaagggccctcacacgtcacccttgggtgtgccagagccagtccagttgttaagaaaaacaagagccggaggtaaaccccttgggaaacagaggtcccagcccacagtactgcgtgggagggagaagtgtgggagtcacctgggctctggggtccctcaggccagcaatggggactgggatcccagcgcaggctgagcctgggtggctatgtccacactgcagagacctatcgtgagcaagtttatggtgcattcctgtgtggaagagtacagcagaggccaggggggtggggctgcagcgacagtgaccaggagtcacccataggggcagggtcctggtgtcatgggccagagcgggccagagcggggacggggggcaaagggaaggagcatttgagcctggatgggggtgtggttagcagagaggtgggaggggagggagagagagagagaaggagagagagggagagaaggagagaagagagggagagggggaggggaagaaggagaaaagggagaggggtgggctgtgaggagagggaggggaagggctgcagagggggaacgtcagtcctgccatgggggggacgtgcgttgttcctcgcccctcccttccaggttttgatggtgctgtttcctgcgtttacaccacagctctttttgcgtctttccttggattccacacagcagccctgtgaggcgctgtcctcctgctgcccggaggaggagactgaggccagagatgcggcaaatttcccaaggccacacggttctgctgttggggcggcactgggcctccctgcccgcctgcctcagtctggccctgtgaagaggtctcttggaactgttaggagcattcgaaagaaactgggcccctgggctgggtacggtggctcgcgcctataatcccagcactttgggaggctgaggcctgagcacttgaggtcaggagtttgagaccagcttggccaacatggtggaaccccatctctaccaaaaaatacaaaaatcagccaggcatggtggcgggcgcctgtagtcccagctactcaggaggctgagacaggagaattgcttgagcccgggaggcggaggttgcagtgagccgaaatcgtgccactgcactccagcctgggcaacagagcgagactctgtctcaaaaggaaaaagaaaaagaaactggtcccctggagcgcaggggtcagcagctaacttctcgccctggcagctggacccgacttggcccagagtgccagggccagtgcccccatctgctctgtgtcgggaggggctgctggcaggaggaggtggccactccaaaatcctatagacggagtccgggggagggggttcgggacaggaacagcccagacctgggatccgcggctgcgcaaggggcttcagggtggtctgggcctggttctgagccctgtgaatccaaggccacaggactgggcctgggggcttctgaatccccaccagtgccttcttgctctcaggagcgttggccaaccccccaggccccagggcttcccctcgtgattctcctctgagagaaacacacccacctacagccgccagccaggagcaattgctgtgcaaacacccgggactgtctcagacggtggatgcaggcagccaagcccccggaaggctccggttgaagggccgggtttattcctccctgcaatgagggtggacactcccccaggaaccacggggcaagagggtgttgggaagagccccactagggtttggggtttgggtgggtggtggggaggggacagtctagggacgtagggctcactagaccaagtgctgttggaaagggggcgattctcctgtgggtgcgttggtcactgtcgcctgtagacatgggagacctgcaccaaaataatgcggagatgggtcaagaagcagccgcccctccccttagtcgagaggggggatggttggtgatcgtgggtggcacagtgacctgtttctatctcactctactgtgatctcagagtgaccttgtctggggtcactggggtgttctgggagatggtttatatccaacagaagaacgacgcagcctggcaggagctccaggtgtcaggggggggcccctttggggtggaggggatgcctcagagccagccgcatgccaccccacaagctacctctctgccgccgtctgccccagagctgcctgccttccgaggcctcagctggtcctgtgcgtccctgcgtctccaggactcttgggtgatggctcaggggaagtttccagaaagcccagccatttcctcctctcctcctgggcttgggtctgaggtcagcttcccagggggcacccaagcctccgtgagcaaggacagagagtggctggagtgagaacgggtgctgctgacctttggcggtgggagggtgtctccagctgtgttcccatttctcagtgactaccaggtgctgtggcccttggctgcagaggggcgtgcaggcagcctctccttcctgtgaagcagcagctgtggcagggccgggaagtagagctcccctctgtgaggctgtggctggggtaggaggagagctctcccttctgcgaggctgtggctgggatggaagtctgccagaggcgggctcagaagaagttccatgaggaactgagggctgggacctgcaggaggagctgagggtgcatggggtctgggggagccccagagatggtcagggtctcctcctgataaatcagacccccccccgccccaccctgccgctgagaggcctggtcctgctctaaacctggagtggatggggagcgtgggctggggctggggggctggggaggtggtgtagccagtgccctgccccaactcagcctctggtcacccttccagggctggcaaggcattgggggtatgagagggccctggggtcccagggtctcagaggacttgccttctctgcgatggccgctactacctgggtcccacaggggggtaagttcagagggggacctggtggaggtcggctgtgggcaggggagccacaggagtgtccaggcccctcccaggccagccccacagattctgaggcagcagtgggggcctcaatggaggagtccactctgcccaccccggatactgcccagccagcctgggttccagcgtgggccaagctctcgtcctggtctcctgggagctgtgtggtcctgcaaggacctcagcaccttccttgctttgagcatccctcccccaaggcctggctgacactcggagccagtatccatagagggctgcaggggcaggcgagggtggacactgatgtccttggctcattcctccctggctggctgctctgatgtccagcagggtgggcgtgaggctcatggggggctccccagccagggcaggacctggggtaaccctatcctgggatgtttgctcccgctgtgtgggggacatgccgtttccatgagtgacagagtcaccgatgtggccagtaccactgtgggcttgaggcatcttctagtttaaggaaatgttaaatttcaagtaaagcttagtgaaaataaggatgcaatttcattccccaacaaaacaacctctatttccttctgtgaaccccttgggagctgcagaccccgagttccggaccccctcacctcactctcctccttcggagccaaagtggtgcctgaaatctgaggccccatctctgagtcctgcactggtcctggcccgaacactcccccacaacgcatgggccacaaggaccctctgggcagattttggggtgtctttggaggtttaggggcaaagcttgaaaactgtggggtcctccctggggaccacatgcccagctgtggggtcccccgaggccgcagagcaagctttctggaagatcctcctcctttgagagccaccatggagcccccataatgggaatggagggtgttgccattaaacatggccttggcgttgcccagaagtctaggtgtgtccctgtcagctgggaggggacagggtaactgtgcggccctccccagggctgaggctggggcagcctccagaggccaggttgtcgctgtcactccccaaatggagggtccccctcattccctgattgcagaaaaagggctccagtaagtctggcttttaaaaaatacagctttgctgacatatgtttcacacaccgtacaattcacccatttaaactgtacagtccagtggtttctagaatattcagagttgtgtcactctcaccataatcaattttagaacattttcatcgtctcaagaagaaaccctctagctagctaccccttccccattcccacggctccccacccctggtgaccgagactccattttctgtctttgtgggtttgctgctcctgacgtttcgcatgcgtggagtcatacgctgagaggccttttgtgtgtcgcttctgttgcccagcgttgttttcagggttcacccgcgtgggagctgcatgagtccacttcttcgtatcgctgagtaatattccactgcggggcaccgcgttttgtgtgaatctgccaccactcaggatggctcatgggtggaaacagcctaaagctggcagaggtttgggctgtttgccagccctgggtcacttttgggctgtttccacctgggggctatcctgagtcgtgctgctgtgaccatttgcgtcctttcgtttcctcctgggtggagattgtggggtggcctggctgggtcgctctgggcccaactctctgagaagagctgttgaacacataccccacagctttttttagtttttatttatttcattttattttttgagacagagtttcactcttgttgcccaggctggagtgcagtagcgtgatcttggctcactgcaacctccacctctcgggttcaagggattctcctgcctcagcctccgaagtagctgggattataggcatgcgccaccatgcccggctaattttgtatttttagtagagacagggtatcaacatgttggccaggctggtctcgaactcctgacctcaagtgatccacctgccttggcctcccaaagtgctgggattacaggcgtgagctgcctcacctggccttattttatatactttttaagacggagtctcactctgtgagccagcacacctggcccatggcttcttttaaaaagagacttctggaagctgagaagtcttttccagggctccacctggcgactgaccactgggcatggtgaggtctgcgtttggaggaagtggggtgacagtgacagctgtgactattggagcacacagtcggtgctgggcaggtgggtcattcctgaaccctcagtgtttccctcctcgggggtcctggccagtcactctcctcctccacaccagcccccagcagctcgcaggctgcctgggcccagccccatcatcagccgctgggcacccacctgagaggtctgaaccctgcccttggtgctgcacggtcacttgccattttcgcagaggggccccctgagcaggaggagcagggccagcatgcacactgcagccaaggtcccaagaaggaagtggcgaggaggaggaggaggactggccccacctgttgccctatgtctcacagcgcagactgggggagacgtgaggatctaacaggggttcctggggggccttcatcaagctgtaatgaatgttgtgccaaacccctgtcaacaccagtgggtacagcacagggttcaagggctgaagaagtgacccagggctagcaaaggagacccggggtttactgcgagccgtggggtggggagggaagtgcagggacgggagaaccgcagcggcttgtagacagcgcaaagttcctatggcatgttcaccccataccctcccctgatgacctccacctgccaaccttcattcaacccaaaactcagggcctcaagcccctgcacagcgcacgttccacgggacaggccaggggctcagaagttcctcatagccaaggaggcaatctccagattggccactcctggattccttagctcaggttgttcttggagtatgcgtaagttactgctatcgggggtgtttaccctccaacaggtgtttggtgggggaggagcctccttgggggtgccaagagcaacagcggccggggtctcatgtctgtggggccactcccattgctttgtgcttagtaactgggtcttcaaggcagccctgtggggtgggaatcatttttatcctgtgatatttcattgtgcacacgtgcagactgaggttgcttaactaccccaggccgcacagttatcaccatggagctgggatttgtaccctggtggcttagcacagagcctgagcacacaactcccacccgcctgcctctctccgaccgcgagtcctccaggaggactggggctctcctcccagcaccaccacggccattgagcccagtgcccaggagaccgaggcctgtgaggtttcacctggggcccggagacaggctccaagtggtgtgggggcccctgtgcccaggttttccccgaggtgccggctcatagctacacatgcccatgggcctgcctgagcccggtgctccaagccacaccccgagtgggcggcctccctgccacgcagtcctcggcctgaggccgccacactcagagccactcagcctatggggtggtgcatgtccagaaccctgacccttgggaaggttgggatggttggagatgcagatgttccaaggggaagtggcgggagcgggtgctggctgggaccaagggattctcaagagaagacgtgagcttcactcaggcacagctcttcagcagcaagagtgttggtcctaaaagatcatgtaaatagaggagggcatggtggctcatgcctgtaatcccagcactttggggagggcaaggtggaaggatctcttgaggccaggagttcaagaccagcctggacaacatagtgaagccccatctctatttattaaaaaaaagttttttaaaagataattttaaaaggtggttgattctgaaataagaatcacctgggaagctcttaaaagcctcaagcccgggcctgccccagccccgctgaatcagtgtctctgggccggggggcgaggccagtcattagttgccaaagttctccaggtgattccaactcaagcctctttgccctagagccgtggctcagcttgaggagcattagggcccctggaggttgttaaacccacgttgtggggccccagcccccagttcctgatgcagctgttggggtgagctgaaaatctgctgcagagcctgcatttcagggttcccaggtggcgccaccagcttgagggggtcttggcgctattgtaacgataacagcagcggtctccagaaccttctgtaggcctctgcatgctcacaggttagaccactgagaccacccatggattttatgtagtagacacacagggtgggcctcgtgcccagtgttgtctaacatgctttgtaagtgcagacccgttcctcccagtgcctttggaggtggagattgtggtccttgttttacaggcagggacagtgaagggactgtgaccgctgttggtgccaggcatctggctgcagctcccaggctctcatccctttgctgtgctcctttgcagcttggagggaatgattttttatccccatcttacagtatggggaaggtgaggctcagagaagtcaaggacttctgtctggggtcacgtggctaggaagctaacagccaggatccgagctcagatttgcccgacttccacccatgccggtgggcgggttttctccttggagaggggccagtcctgccccgaggtgccatctctgcacaggtaacagcatcagccaggcaccctgcaggccaccccgcccctcgcaggggtttccactaaccctgctgtctgccaggccagcctgtgggccccaccacctcctgggtgagcgttcccaacatcagagctatgcagcaggcagtgaggggatcatgggtctggcaggggcaggactgggagggacacccttatgtgctgtgtcctgttcctgcctcccctgttcacgccccacagaggagggaggaagctggggttaagggcaagggacagttcctgctctcccaggccccacaaggcatgcaagaggctaaaaatcagaagccagcaggacgcccaggtctcctgagcctgtgcgtctgatctgctcacctgggagacgatggtgctggcatgggcgggtcagaaggagcagcaggacccttccacggcccaggacaagccagccctcaggggccagagcccccatgctgggcacagggctgccaccttgggcggtgccctccctagctgccgcacttcagagtgagcccccatccctggcgtctgccctcctgagtgagtgagggaggggccgggtggggccggatcgtgatcagagccggagctggccttcgtgagtgatggccttgcctgctctgttgcccggtgacagtaaagaatattcctacagagtgtgggttttggaagcagacatgcctgtaggggtcctggctcagacgcttacctgacgttagcacctgggcaagtggcttaacctcatcactcggtgttggcatctgtaaaatgggcacaataacatgctaatgtctccctcgcggggctgtgcgagaatcgattcagtggcccgtggaaacccctagcgtggcccggatgtgtggtgagtggccttggaggcgtcagccttcatgggaggtgggagagctttgccgtcctgggtgaggtcggggtaagagaaagcagagtcggtccccagcaggtgtggctgggaggatgctcagtcactcccaatgttcattgggagtcactgtgctcccagtggacagagtgtcaggtagggtctacagatgagccccacttcttagccttgccctgggtccacaaatcagccagagttactgggtgtggggcttcctcaatgtcagggatgggggtgaacagaagggctgggtccggagtctccaaggctccttccaggctccagggctaacgctgtggggggggcccaggtctccacctcttgttgctgtgtggccttgggcaagtgactcacttctctgagcctctgtgtgtcatggggaagtgactccatgcctgctgctgtctcggtatttcacaagtgctcagcaaatgccagcagaccgtgtccttcagacgcggcagctggggttcctgactgctgggctactgctgcggtggctcatgaaggtttttctgtccccactacaaaacaaaaggcagctgatgcaggagttttagtaaagataatgtatattttagtaaagataacgtatatttgatttaaaagaatgctctttattctgagatttggtccatcccatctttgtgggtttttttttttttaatttggatgtgaaaaagacattccctttatttgtaattgcccaaagtcggaagcagcgaagatgccattaaatagggaagtggataagcaaacagcggtgacgacgcggagcagccttcaatgcctggtgctcagcgggataagcccgtgtgaagaggccacatcccgtggggttccaactagatggcgttctggaagaggtggaactctggagacagtgacaagatcattggttgcccagggttgggggggagggagggacagacaggcggagcccgggggatttgtagggcagtcatactactctgagatactctaatggcgagtatatgtccggatgcctttgtgcacacccatggaaggcgtggaccctgatgaaaaccatggctctggttaataatgtatctgtcttggcccatcaattgcgacagatgtaccacaagatgctaatatcaggggaagtggggggcgaggggatacagtatatgggaactctctgtactttccacttatttttctctaaagctaaaactgctctaaagtattttttaaaaagaaaaagttaaaaaaagaccacatgaacccatgtggagagtggaagaagcaaagtcaaaacattgcccagagtccatgctggaagccagttggggcgtggctgctccactcagttgactgtctgagtcttgtccccacagcacagattggtggaccttcagcccccacactggaggcactggccttccgaatcccttctcacctgagtggggtcaagacgcacctgggtcctatttgctgcccacctctggggccagctgctgttcttggcacctcccaccctagggggatggtgtctgcaaacggtgcttttggcctctggcatgtccaccctgccacaatcatcttcctcaccctcatctgcagaccttctgacaccacctctgctgacccctctccccttgtccttctggggagatcacgaacctgccccttcctggcattccccagccagtcctccctggggagcaggactgtgcatcgaggccaggctcaggtcttcacccacacccacgttgacaacgtacgagtcccctctgagccttagtttctgtggctggaaaaatgaggttaatatcactattgtgagaatcttctcataaacgcatctttccttcccctcctccttaaaatttttaatccttattgagctattatgtgcccatagtttaaggtgacatttggtgctaaaaacaaaatagagcttggagcaaaaatcatgtgtgtcttcagagactgaccaccatgtttttaagtgggtgtgtttgccgcttcctgagctcatggtcacttttagccattatctgttgactcctactgtagtagatggagattttcaatcccttagacccccattctccccactcccagctatctcaatattgctatattgcattttttgttgttaaatccatactcagtgtttttcattatgactatgtaagtattgcttactgctgagccaggcaatggagtctgattctgtacacttctcatgcataacttgagttagtaattgtctcgctttttttcagttgcaattttctttaaatcactggttcttttttcaccttccaactgctcagtgaaacacctctcaaatgcagcttgcacttgagcaaacctgccagattcagtaatacacacaaatgtacaaatacgtttgtttattgattgattgccttcttcccaattcttcccccattttcctgggtatttccttcacttctcctgtgtttgatcttctcatgtcttctttcatggtttcctccctcatttgggtggagcacatcctggagtagcttcctaagaaaggatgcatggaagatatagtttctgtggcttgattatttgataaattggtatgcatagtatgctaggttgaaaatcatttctgctcataatttggggcggtgggggtgcagtttcccactgacttctaggttcagtgttgctcttgagaagcccatgctgttctgattctcagtccttcatttgtgatctgtttttatgtttctctggaagcatttagggtctttgtcatttgagttctgaatttcatgccactgtatcttggttgggtgtctctcccctcattgtgtggtatctgtgagccctccagtctggacgctcacacccttccattccaggagactttcctgtttaaatctttgcccattttccttccctgctttctagaactcctgctgtttagagttcgggtgttctggatgagtcctttaattttcttactgttttgcctacctacctctttttttgatctagtttctgaaagattttcttgatttttatcttccagcttatttaccaaaatatttcttttagctattatacttttaatttccagaagctctttatccctggtctttcctttttaattgcattttattctcattttgtattttatctctgaggatttaaattctggtccctcgatgtcttctcccacctgctgcattttctgattccccctcttttttcccacttatttgtaagttttggtcttttttgtccctgccaaggcttctccttgtgtctgaggaccgttgactgttcatatattaaagtgaggtacgaaaaagctgagtggaaacttgtgtgtgtggcagccttcgcttctggctacagtctgttttttcagaggaattttccacctctctcctggataaagtaatcctccctgtttgggtggggtagcaagcagggtttttggttcagaatgagcactttcccagtgcctgctgaagcattggcgaaaagacgaaaaaaagagcacttccctggcagtcctcccccaccctcctctgcctggtgggcttgtctgattgttggcctagaaacttttcttcctgggttctgcagaaggagagaaaactctcttcctttcattcattatttatatttctcagcaatctggctttttatcctttttcttggtttgtcctaactttaagagatggagtcttgctctgccacccaggctggagtgcagtggcacgatcaccgcagcctcaatctcctgggctccagcccctcctgggctgaagcagctggactacaggtgtgcaccactaaacacactaatttcaacaaaattttggctgggtgcagcgactcacgcctctaatcccagcactttgggaggccaagacgggtggatcacttgaggtcaggaattcgacaccagcctagccaacatggtgaaactttgtctctattaaaaacacaaaaattagccgggcatggaggtgtgcacttgtaatcccagctactccagaggctgaaacagcagaattgcttgaacctgggaggtggaagttgcagtgagctgagatcatgccactgcactccagcctgcacgacagagctggactctgtctcaaaaaaaaaaaaaaaattttggtagagacagggtcttgctgtgttgcccaggctggtctggaactctggggctcaagcagtccttctgcctcagcctcccaaagggctgggattataggtgtgagcctccacgcttctcctgtccttttacttcattatttatatagttaatcacctcgctagagactgagaattctgtgtcctcaggctgccatcacaaaataccacagacagcatggcttaaaccacagtttatttccttgcagttctaaggctggaaggtcagggtcaaggtgctgggagagttgattgttctttgttttcgtgggcttgtccctctgtatgcctctgagtcccttcatagaaagacaccaggcctattggatgaggacccccagtgccttcatttttaactcagccacctctttaaagaccttatctatctccaaacaaggtgacattctaaggtggtagaggatgccgggggtgggggtgggtaggactgcagcatatgcattttggagaaacgcaattaagcccatagtgaattccggcagacataactattcagaactccactaagattaaccaaaaaaaaaaatttatatatatatatattaaatgatgggcggggcgcggtggctcacacctgtaattccagtgctttgggaggccgaggcaggcggagtacctgagggcagtttgagaccagcctggccaacatggtgaaaccctgtctctagtaaaaatacaaagattagctgggtgtggtggcgggcgcctgtagtaccagctactcgggagtctgaggcaggagaatcgcttaaacctgggaggcggaggttgcagggagccaagatcacacactgcactccagcctgggcgacagaatgagactccattgcgaaaaataaaaaaaatctggtgttggtagtaacgatgtaggggacatggtctctcttatagtcttttctgagggtaatttctgaaaaaccataaccccaaacccttgcttttgaccaagcaatttcattctaggaatttatcctaaggataaattttaatgtcatatgaagattagctgtgaaaacggaggagcgacccgaataccttgcggcataggattagcttgtaatttatgtgcttgtgaaacggccgtgcaatgaatggggctccgtgcagccggggacaaagtgggtgcagagaggcaggagggacaggcacggggcatgggggccacccgtggagaaagccctggagctttgtgaatgccgtctgttggccagcgcagggctctcactgtcacgttggacctgtgcccagggtctggtccctgatctgtgagggtcccagagggggcacaacaggcgaagaccccaaagacaggggtctgttccggggcatcctgggcagagagtgggtataggggacagaaacccagccaaagcggctcaagtggaagatgggtgctggtcgatatgggccagcctgaggggtgtcagagccgggaatggggaggccatcggaagcccacaggagccacttctgagttccctcagggtggaggcctggccgccttcccagtggtgcctgtttcttcctttctctgcgggttggcttctctgcgtccctgcctccaggtgggaaggggtccctgcaggcttcgtctcctcccagccgcagagccaccaccttgggctcccctgccccaggcccaggctggtggatctaattgggatttggtaagctgtgacgggaggggggctctggcccgtgcccacccatgggaaacgggccctataggaggcaggtgaggcgggccagagtgaatcagtggtccagtccagaacggaggctgtgacccgccagggtgggagaggacgggctttggggttgggcctcagccccgagtgggcaggcacagcatgggactggctgagagacgctctgtgtgggcttcggatcagctcagcgtaggtttcagtcccgctctagctgggtgacactggggttattttacctgtctctgcctcagtttccttctttaagtggacacagtaagagtgctgagctcccggggtagtgtgtggatgaaaggtgacctccgcctgcatggagccatggaaatgtttccccttttccctccactggactctgggacagtagtgggagcctgggtgggggcttttgcctgccagaactcatgggtcggggccgcacatgagtgagtgtctcctgggcagggctgtggccatgagctcaggtctttagtgggctccttgctcctcccagcaaagcgttcaccttgccccaccctgggacctctcaggtgctctttggaggtgttgacctggggcatctgtccatggccctgaggaggtggagcacctggtgagggctgcgctctctccagctgggaccccgggctccgaaactaaagttttgggaagagtgtcagctgacgggtggggcttcagaggaggaaaagaaacaagggtgtgagatgtgcaggccctggtgagagacaggcagcagcaagagcctgccagccatacttacccacaaggccggctgaggggctgtgctggcccagggctctggctttcggacccagacaatgctgctgaccaagcaggccaggcgctgagtgactgtcccagcggatctccgagcagggtcccttgagcattaggaatgcagcttctcaggccatgccagcctggcagaacaggaagggaggggtacagccctgtggggacgcagctgtggctcgagtgacacctgctggcggcctcatctcatccgctctctgcgagtctgtggtggttctagtcggcgtttcacgggcaaggtttagggggtttgttcctcagctggggtccacccacgtcttccgacttcctctctcagtgtttgggccatcggtgggtgaggtggctgcagcagagccgggggctcctgtgtgcggaggcagcaccgacatccactgcagccacaccctcctgagagggtctggaaggaagcagggcaccctgcgaacccaccttcctctggggtgtcttccggggccccagggccagggcaactggagcagacaggacattatctagctctgggggccgtgctgaccgagccagaccctcgcggcggcccacagagatgggcagcttgcggggcagctttgtccccccgacactgggtcctgtcttcatgcacacaagccgcctgttctgcaggtgaccagttaggttcgatgggacaggctttgggaatcagcggcttgggtttgccccccacagccttggatcttgaccttgggagaggcctccaccacgtgccctggggtggggtgggggcgggggcgtcctgcatctgcgccgttcagctcagttgccctcagccgcggttggccgccaagcactagggtgttgctggcacagccaaggaactgagcccagactttttgtttacttaatcaagcataaatgcaaagggctgggggtggccgagctgatggtgcaggcgtgtgcgctcaatagctccgtcacagtggctgtcgctttattgagcaaccgagggagcgagggagaggagcaactgcggcgagcgtggaggcccagcccgcagagctcccgtctgggggaagccctgtccctcactgcctgttcccggcgctttggtgccctctgctgtggggtgatgaggtcaggctacgcaggcagtcacggcagaactgaaggccgtgtgctttcgtgtagccaaaccccttttcttactgatatcagttataaaatcagagctatgttcccaccaggggcaaaaatccaacaccgtgcagttgcaaacacggggcaggaaagactccggcgaccctcattacactgtgccgggaggcatggccagagctccgtcagttcatgcgtcgcttaaacctagaggtgggtgcgattgtcaccctatcttacaggtgtggaaactaactcagctccgagatgtgactgaccttccccggccatagtacgcgcagcgcagtggggcctggacgcggctgcctggtttcaggtccacacgctccccagcactgccggccaggctccagcatgacgggggtcaggcctccacaaggccctgtcggccacgggagtttgctgcgagatcctgggtccctgactgtgttctcaaagcccctaggatttgaccagccgttcttttatcttaggagtcggaaatcttacatctgtatttttatttcgctgtgcttccgccccctgctctgctaccctcacctcactacgttgttttattggcatagtataaaattcacattttacaatatgcaattcagtggcactagcacattcacggggttctgatcatcagctctatcaggttgcaggccactttcatcaccccaaaaccccagccctgtgagcagccattcctgctcctccccggccatcatccgtctgtctctggatttacctgttcatgttatgggggagagagagagagagagagagtgtgtgtgtttgtgtgtgtgtgtgtgtgtgttttaaatgaaggcttgtggtacggggtctgttctcattttgcgccggtgttgggcctgaagtgtgtcctgtgtgcatgtctctgacccccggagtcatgggacccagtcacgtgctgcgctgacgtggagtccgaagttgtaaagacaaagctgggtgagaccctagtgactcacaggcccttctgctctcctttccagatctgggactttctcggggacaaccctgggataacagccaccactggagtctatgcttgaaggccattccgcctcgtggtgccctgggcgaatgtcgagggaaagcagactccctgtgcccccggggttggcttcttgctcaaggattccagatgctggcctcctgggtggtgttcccatgccgagctccagttgggggtgtgaccagcaccccgtgcgggggctgctggtgttctgtgttgtcagagcagtgctgttttgtgtttttctttaaaaaaaaaatttttttttgagatggggtttcactcttgttgcccagactggagtgcagtggcacaatcttggctcactgcagcagcctctgcctcctgggttcaagcaattctcctgcctctgcctccccagtagctgggactacaggtgcccgccaccatgccctgctaatttttatagttttagtgaagatgaggtttcaccatattggccaggctggtctcgaactcctgacctcaggtgatctacctgcctggcctcccaaagtgctgagattataggcatgagccaccacacccggcctccttttaaaattttaatttttaatgagatggggtctcactatgttgcccaggctggctttgaactcctggcctcaaatgattctcctgtctcagctgtgttttgtgtttgctccagccctttgctggtgaacagacacgttcactggacacctaagtgcctggaccgtggagcacagggtgggggtgctgttcatggccggaacgccagaggctactggggagacacaggagcgaccaccatgatccacagaccaggactgtacagagcacttgaaggggcggcctcaagaattgtgccggaacagtgaacatgagggctcctggctcccctctgaggacaagggttaggtaggaggggacaggggcagggctgaggtgggtcggcttggttcatggcagggcttggggagtgaggagagtgaggagtaccgggacccggggtccctgctctgctcacctgctgagaatgagccagtgttggcctctggtctccatgtctgtagagaggcttctctgcttcccctcctgtagaatgggaacgacaacatccagccatagagtgctgagaattcagggacaggacacagcgaaggtgccccagtcccccttcctgaccccagcctggcgtcaccctctatccaagggaagaagcagggatctgcctacgaatctctcaggatgtgaagtgtggcccccagcctggtgcctttgcagtgggcccaggacggagggtagccttggcacctggggaggacaggtgaacctggatccagactctgcctcaggtgacagccctgggttctaacgctgccgcttccgcttcctggctgtgtgttcctagggcggccacagcccctctccagcctcagttgcctcacctgggaagagaggtgcagggcttttgcgatgatcataatagttaccatcaatcgagcattagcgactttccagcaccttaaggcagttaccctgtgatctgcacagccgccctcccctcgttgcagatgaggagactgagtcagagagaggttaacccagacattcccaaggttgtagattgagaaagtgttgagttgggattcgaactcaagtctgtctgaggcctcctagccccttcctgaccccagagccttccgcacctcatggctccaccagaccctgcatgaagctcctggtgggcaaactgggcaccttcactttaaacgttgagggaaaaatacacataacctacagtgtcccgtctcagtcatcttcacgtgtgccgttctgtggcattaagcacattcacgctgttcaaccgtcaccagaacttttccatctccagaactttctcatcttcccaaaccgaaactctgtcccactaaatgctcactgcctgtcctccctcctgtcctccctcctggcctccctcctggcctcccgcctgtcctcccgcctgtcctccctcctgtcctccctcctggcccctcgcaccctccgttctactttctgtgtctatgagtctgatgactccagggaccccaggagagtagaatcccacaggatttgtcccttccagcctggcttattccactgcatgacggcctcaaggctcatccaggaagcagcaggtgcccggctttccttcctttttaaggctgaggaatattccactgcatggggggaccacgtcattttatccccgatgccttcacttttcagctcttgttcccatctgtatctgtaagctggagcatcccttgatggaggagggatctcagggagtgggtgagcctctcctctttccagggtcatcctccactttgcgggggcagcccaccttcctggagttccagctgagggggggcccaatttaacaagcactttgggaggttgaggtgggcggatcatgaggtccagagattgagaccatcctggccaacatggtgaaaccctgtctctactaaaaacagaaaaaaattagctgggtgtggtggtgcatgcctgtagtcccagctactcaggaggctgaggcaggagaatcgattgaacctgggaggcaaaggttacagtgagctgagatcgcgctactgcactctagcctggcgacagagcaagactccatctcaaaaaaaaaagggttgtccggttacctgtgcatgccaggtaatcgacaaatgctttttatttttatttatgtatttgtttagagacagagtcttgctctgtcgcccaggctggagtgcagtggcatgatctgtgctcactgtaacctccacctcccaggttcaagtgatcctcctgcctcagcctcttgagtacctgggattacaggcacccatcaccatgcctagctagtttttttgtatttttagtagagacaggatttcactatgttggccagcgtggtctctaactcctgacttcaggtgatctgctcgcctcggcctcccaaagtgctgggattacaagcatgagccaccccacccagccaacaaacgctttttaaatataagtatgtcccatgcagcgtgtcccctaaagaacgatgtgttgttcatctgaaactcagatgttaccaggcgtcctgtgtttgatgtgacaaccctgtccacagccctggcacggaaggggcagtcgctgagggtgcagtcttggggtgagggtccctgtgatggcagcctgtgtccagcttggactggccatgggaaggccactgtgtgggcgggtctggccttggtaaagggccaccccctgagacccaccgagcctagttccagtttcctcctcacaaccccagcctggaagggcgttcgggtgtggagcaagccacagggtttccccccagtgtggttcgcattggggtgccgggtggttccgagtcagctgccccctctgcctcctccccgtgcccatcacaactgcccccagtgctgggacctctgggcaagaggctgaggctgacactctggaaaggtgtgcagtggtgttgtggggtccctggggtccggtccacatacatccctgcttgcccggctcttggcatcctgtaggagaaggtgccacagcctagtgggtgggagggcaggagccccatcactggagtcggaggttgcttttgatgttgatggcagtgaggttgcttctgatgttgatggcagtgaggttgtttccgacgttgatggcagtggacttgcctccaggttagctgcctccccaagcagctttgggggtacagaatgaccgaccagtgtgggatcccaaacatcctatccccttgcctgaaagtgggacgaggctgaagctgacctctcgctgagaccacctcattcggccccgtcctctgctgccacgtccttcttcctggtaccccccttctccagccccaaataagtcacgttcacaggatccccagctcaggctctgctcctggagaagctgccctcagaggctgcgttgccgattcctttttcaacggagaggctgcagcgtgtggcaggtgccaccatctcttggagtgaggcagcctcaggggctctgtccggggctgcagcctgattggtgatctttgcacgccccttctctgccctggctgggccatatgccacatggggcgtgaggggcctgcaacacaagtccctctgggggctcgtgatgtggttgagctggaagatacattctggaacatctaagaccccagccactgtcctgagtctggatgccgcgggtgccgggcatgggggctctgggcccgggccctggggtgcttggaggtgctgtggaggaggggtggggctggcttggagcccatgggattcgattggagggtgggagtgtgagagaggagagagcgtgaacagagccttggacagaaaagcctggaaggatcaggaccctttggggggtggcgggtgaaggtggcggggtgtggggataccctggggacggacaggtcacttgaggctacaggactttgaattccacacttagtttggatcccaccctctggtgtggggcgtgtgagtggagggggcctggctagggaaagggggggggcagtggggagtggagggctgcttcgctccgagagatggcggcacctgccacccgctgcagcctctccgtttaaaaagtgactggcagaacaggagcagctgctgccagctcctgtccattttgtcctctgaaaagagaggtcggggtggccaggtcctcatttttcaggagacagaagagagctgaatttttatgtcaaatctcttggttttgagttgttagcaggtaattccaatgttttatgaaaacagcggtgggacgaataaaacctacgtatcagtaagtctcggcctccaggcctctggtctgcagcctgtgccaccgtgagttggaagagtggtggacggactctgttctccccgcaaaagccctttccagggtggcacatccacgccgttagtgcccggctgcagggagaagctcaaatgacaaatgaggcttgaggtttgccgcgttgggcgtcgtgctgaggcccagccgtcctcacggtgacctttggcatgaggagtcctggttccactctacagacgaggacactgagggccagcggggcccagaaacttccctgagtcacagctcacagagcaagacatgggcccaggtctgctgaccgggtttcctttttattttattgtggtaagagaaatacagaatgtaccacgttagctacttttaagtatacaattcagtggtattaattaccttcacggtgttaggtagctgtcatcactgtttacctccaaggctttcccatcatcctactgcaaaactctgtccctattcaacactgaccccacaaccctccccagcccctgcacccaccgcgctttctgcatctctgagcgtgactactctaaggaccccgtgtacatggaatcatccgatatgtggccttttgtgcccagctcagttcactctgtgtaatgtcttcagcgttcatccacattgcagcctgtgtgagaatgttctaccttttcaaggctgaatgatgttccatcgtatggacggaccgcatggcgtctgtccatttaccaggtgatggacttgtgggttgcctttccctttccagccatggaaacagtgctgctgagaacccaggtgcacaggcagctgctgcagtccccaccctcccttcttctggggatatcaggtctgctgattttatgtctgaaggtcatgggccactgtcccttccctatgcccttgaatctcttccatggtgtgtccactgtgtggtcttcctggatacctccagcaatggggagctcactgtctcccagagcagcccattcagggcttagtgtgtagcccacacctgtgtccctgtagctgccactcattagcctcctaacccaggacgcgtttccttgtgcccttggcaggggctccgtattcactctgggttctgtggtcctcactctgcaccacagactctgatgcctcccttctccctccccgatttccaagagagaacaagatctagccatgagaagcaggatggaggtgtcttcaaaaacagccactggtgatagaagttgtaggaaggtaccatggtctgtctgcctccacgcaaacctcaccaggtttcatcaagatagcacaccaggagtggtgtggctcatgactgtaatcacagcacttctggaggctgaggcaggatcacttgagcccatgagttagaaaccagcctggccaacataatgagactgccatctctacaaataatttacaaagtagtggagcatggtggtgcacgcctgtagtcccagctgcgcaggaggctgaggcaggaggatcgcctgagcccaggggttcctggctgtcgtgagctgtgatggtctcactgcactccagcctgggcaacacagcgagaacctgtctcaaaaaacacaaaacaacaggaagctcagacaggactgtggctgcttcaaccctgtccgtggccaggctgtgccccctcctctgtcgctgggcactcacccctctctcattttctccccagggacctaccagggccagtgggtcggtggcatgcgccagggctacggcgtccggcagagcgtcccgtatggcatggccgcggtcatccgctcacccctgaggacgtccatcaactccctgcgcagcgagcacaccaacggcacggcgctgcatcccgacgcctctccggcggtggccggcagcccggccgtgtcccgcgggggcttcgtgctcgtggcccacagtgactccgagatcctcaagagcaagaagaaggggctgtttcggcgctcgctgctgagtgggctgaagctgcgcaagtcggagtccaagagcagcctggccagccaacgcagcaagcagagctcctttcgcagcgaggcgggcatgagcaccgtcagctccacggccagcgacatccactccaccatcagcctgggcgaggctgaggccgagctggcggtcatcgaggacgacatcgacgccaccaccaccgagacctacgtgggcgagtggaagaacgacaaacgctccggcttcggcgtgagccagcgctcggacgggctcaagtacgagggcgagtgggccagcaaccggcgccatggctacggctgcatgaccttcccggacggcaccaaggaggagggcaagtacaagcagaacatcctcgtcggcggcaagcgcaagaacctcatccccctgcgggccagcaagatccgcgagaaggtggaccgcgccgttgaggccgctgagcgggccgccaccatcgccaagcagaaggctgagatcgcggcttccaggtaggagggcgagggggcggggggcccttcttggtgcccagaaggtgtttgtgagcccagtctgttcagtcctgctgtcgctcaaggccttgggctaggcccagtttgaggcctacactggtgtttctcaaactatgcccccatggaaccctagggttccctggaggttctcagaggtgactgtggtcaggattggtgcagaggaatggttgatttgtgggggtgtagaggcctacagcctccttaaactgagcagtgtctctttgttctatatgctgatgtttagtgtaaaatattggtgaataaagaaggtggtctgttccgttagtacaatgcattggagtgctgctgtgctagctagtaggggtgacaccaccaggtgggggtggcaggtccagcactgcagggctgtgggaggtggctaggagctgcctcagctacccagacctgcgatctagattcttccgggaccctgggagatggcagcagttgggaggccaattccgagtgcctggcaaggggctcatgcaggcctggtgctttgggggcagccgctgttctgaatcggctgcatgacttacggaatttggggtccagtggggcctctaggccctaaggaatgggtccagaggtgactgtgggtgggatcgacgtggcttttgggtacctttaggggtgggtgcaggatggggcctttagggctggaggaaggtgcccagggtctggctcctgtggaggtgagtgtgtgagccaggggctggtccccgccaagctgtggctggcaggagccacctgctgcaggttggaggaagttcccctgtggaaagccacgggccccacaggtaggcatttgggttctaaagtaactgaggttactaaagggctttgtcaagacttgggaaggcatttcagaaaacccagagcctttgctttccaccggcatttgctttcctgggccagacgagggcccttggggtagcccggagagtcttggggcagggatctgggagctgcgggggagtccatgggctgcccaggcaggaggcttcccagggcacgttcccctgcgtctttctggatggctttgtgttctcagccaggggcagacctggagttcctccaggaaagctcccagcctgggtgcggctcagcccttcccgacctgtcgctaattaaagttcagcccatttggttctgattttcagtgacctttgatctatagtgaaaaacaaaaatggtggctgcggtttgttggcagaatatgttgctcgcgacttctattgttttagagggaatcaagatcgattccaagaagctgaaagaaacatggaattactacctgacaccgaggtccaaactcgctgtttgcccctccctgctccctcagaaatgttcaggcagggaaataaacagcagcccacgtgtgactactcatctggggttttaagaacaaattagaatcagtagttattttttccatcataaatgtaatacttgttccacaggaaaataaataacactgagaagggagaaagcagcattagctctggtctgatggatcggccgagcgctctccttggcattttggtgaatttctttcctctgtgttttgtttttcgaccctttctattttgtatagttgcttttttgaccctagtggacaagaagcagttttgggggctgggggacagcggcgtcacggaattgaggaagctcgtggcagcttccgtgtgacctgcagattggttcatgagttcattggttcatgagttctcagcagctcgaggacttagtggacagtgccctgcaggactgagcttatttcacagccttggcactgggtggatgtctgtaggactggggggacctgcgacctgagatagttttcagacgaattcccctagacattaagagaagcgggttcccattaaggagcccaggtggcatggacagggctgggggcagcacaggtgaggagctgctccaaggcaggttgtcagcctctctggacctcaacttctaatctctgaaggcctgtgaagcttaggtggtaacagctcccttcctggggcatccaacctcttcctggggttttggggggcccaggggctctgtggagcattgctgggggagatgcctacgtataagtgggcatcatgctggggtgaaagcccaggggctgggccgggagggcgagacctgagacccatgcaaaggagccggggccagggtggggtgggcacggctcctactgagtccccagcccttgggacgctggccagccagccttggcatggccagcatccctgtgtctcctggggaggcattttacttaatggacagaaggacagacagacgggtggagggagtgagagggaaaggatgggtttgcaggctggtgcagctgtgaaactgacggagcccaccccctgccaaccccccgacagcctttgaggcccagcagcccatgctccctggcaggttgcggcttgccttctgcactggcggagtccgagctgactccgtcccttcgcccgagcgggcgtcgcggggagggaggtggttcctgcttccgcatgcgcacaactggggtcttggatcgtagctcctttgtgagaaagggactggctcacccgccatgtcctgctgtccacacacacggctctgcacaccactcacggcctcctcaccgctgtgcacagccagcacgtccatgcgcacctgtggagccagggtgtacggctgcacaggtgcgcctaggtggaggtctccaggtcactggaacttaaagtgtccaagagaggaggtgtcaggcctcaaaggggcactaggaactggcgtctcacccttgcctctgagacccgcagacccacatccagcctgggctgtcgtggctgagtgccccaggccggtcgcctcccctctctgggctgggttccctgctgaaggggggcggggctccctgctccctgtagtggcctcggcgtggggccgggcctgtggttgcaccatctccattggtgtcatcctcccccagccctgctgtgacaggcatgggaggcaggtgtgtttgtgagctcactggtgcgacccttggaggtgggaggctacacagtatttttggtcatcagaggatggaggatgagcctgcattctgggctgcagggcctggagctgtgctttctcaatggacttagaatttggggaaaaaaaaaaggaaaaaaaggagggaagagagtctgccgccctggagagtgaagctgatgaatggctggctcaggcgcccacagccccagggccaaccctccactggagcatgagagcggccgcaccacgtgggctgaggagtgactggccccgggcgggctcctgccactatcacagtcgagagatgcgagggagttgtagggcagctggcactgtcctcacccgccccggccggcagggcttgtcaggccgcgtgaagtgagaaacggtttgcggtgtcctccgcggagtgccgggcccttccccggagagcagctctgcagggaagggaagtgggttttcctggaaaacggggcgggaggtgtggagctcggagagactgaggtcctgcactgctaaggggactggccccggccctgggctgggagagggggagctgggactcagcccagggtggagtcaccatgggttctaaggcacaggatggataccctcctcggccatgtgacatcattgtttgggatgcgcccagtgttcggagccttcgaggctctccaggtgcctggagggcttgtagaaatggtggtcaggccccacgccccagactttgtctcatcatgtctaggtgggggccccagcacctgcatgctgacctggtccccaggacgcagatgctgctggcctgagcgtccccggctgtctcctgtctggtgtgggtgctgccgccgggaggccttggccccacggggagatgaggcatcacacacgcagccacaacacgggtcggaggcagagtccagcccagcaagggtgtggcagtggagacgagggaggggtgaagctggtggttccaggaggttccgtctgggtgagaccctctgctgcagagaggggcgggagccagtggccccgcggagctgaggtcatgtgagaaggggctgcagtctcttctgtgtcattctcctgtggggtcagcctcaccccagtgggaactcccactagtgaaagtctgagatggccccatcctgccgtgtcgcctagcagtgcaccacagggcattcggcccctcctgccttcacacactcaagacgttttcatgactcatctcctgccaggagggcacacacagggcacctttgcacggcgctgcctgtcctgaatgcctcgctgtccccgggtggacataggaattcatcacagtcctcctcgaagcccgggttgctctagaagatgaaactcaattgctgtccttgatctgctgctcactagaggccctgggttgggaatcggaatgtaaatgtgtcacccatcctagagaagaaagtcctgcagtgtgacatggcttcattttcaacagctgagaccctctgccacaacaactctcacagccccctcatccccacccgccgtggaaaccaaataaaagcagaaagctctggtggaagccgagctgggggcgatggggcagattgtagcccgtttctccttcccgtccccctccccactttggcccctgggacctgctcagaaccccagggtgaaggagacatctcaagtgtgacccattggctgcttctgctgcgggagagggagagacggggtggcccatgtgaaagctatgcctcctgcagccccgcaaagcctgcaaaagctcagccaggatttgatgggattgtttctgctgggaccccctctctgctgcgtctcgtgcaaacgttgctgtgaagggatgccaaggggttttgcaaagcagaaacccaagctgagaggaggagaagggattgttctgggggcaggcccaccttgattcagggggtttcgcttcattgtgctttgcagacgctgtgtttttacacattggaggtttgtggcacccgtgcatggggcaagcctcttggtgccgttttcccaacagcacgtgctcacatcgtgtgtccgtgccacactttggtaattctcacggtatttcaagctttttcatgattatatctgttcatggtgatctgtgatcagtggtctttgaggttactagtgtggttattttgggatgccacacatcgcacccatataagacagtgaacttaataaatgctctgtgttctgaccaatcctccaaccagccgctcccccatccctctctctcctctggtctccctattccctgagacacaacagtattaaaattaggccagttaataacactccaatggtctctaagtgttcatgtgaaagaagagtctcacatctctcactttaaatcaaaagctggaaatgataaagcgtagagaggaaggtgtgtcaaaagccgagacaggcctcatgcaccaggcagcctagttttgaatgtaaaggaaaagtaattgaagaaaattaaagtgttactccagtgaatacatgaatgataagggagtgaaacaatgttattgctgatatggagaaaattttagggtctgaagagaagatcaaactaaccacaagcctgatccagagcaaggctcaactctaatgctgtgaaggctgagagaggtgaggaagctgcagaagagctggaaacgagcagaggctggttcatcaggtttaaggaaagaagccgcttccagaacataaagtgcaaggtgagggagcaggttacccagaagatctggctaagattgttgatgaaggtggctacactaaacagattttcagtgtcgatggaacagctttctattggaaggagattccatctaggactttcatgctaggaagaagctaatggctggatttgaaggacaggctgactctcttgttaggggccagcacagctggaaatgttaacttgaagccagtgttcattgaacattccaaaaatcctagggcccataagaattgtgtaaaatccactctgtgctctagaaatggaacaacaaagacctggatggctgcacggcttacagcatggtttccagaatagttttaagcccagtgttgtgatctactgctcagaaaaagattccttttaaaatattactactcaccccggagcgcccatggagatgtacaggaagatgaatgttgtttgcgtgcctgctaacataacatccagcatgcagctgtggatcaaggactaattttgactttgaggtcttattatttaagaaatacatttagtaaggctgccatagatagtgatttctctgatggatctgggcaaagtccattgaaaaccttctggaaaggattcaccattctagatcccattaagaacattcgtgattcaaggttggaggtcaaaacatccacattaacaagaatttagaagaagttgattccagccctcatggacggctttaggggctcaagacttcagtggaggaaggactgcagatgtggtggaaatagcaagagaactggaattagaagcggagcctgccgatgggactgcattgctgcaagctcacgaccacactcgaaagactgaggagttgctgcttaggggtgaaacgagaaaatggtttcttgcgatggaatctactcctggcgaagatgctgcagacattgttgaagtgacagcaaagatttagaatgttccttcaatttagttgataaaacagcagcagagtttgaggggattgactccagtttccaatgaagctctaccgtgagtcaaatgctatcaaatagcatcacatgctacagagaaatcttttgtgaaaggaagagttcattgatgcagccaacattgttgtcgttttttttttgttttgttttgagatggagtctcgctctgttgcccaggctggagtgcagtggcacgatcttggctcactgcaagctccgcctcccaggttcacgccattctcctgcctcagcctcccaagtagctgggactacaggcgcccaccaccgtgcccagctaatttttttgtatttttagtagagatggggtttcaccgtattagctaggatggtctcgatctcctgacctcgagatccgcccacctcggcctcccaaaatgctgggattacaggcgtgagccaccgcgcccagcctgttagaagttgccgcctcagccttcggcactcaccacgctgatcacccacagccaagccaagatcctccattgaaggctcagattattgttcacattttttagcaacaaagtattttaaaattaagatttgcaattttttaagatataatgctatagcacgcttaatagactacaatatagtagaaacataacttttttctttttcgtgacagggtcttactctgccgtccaggccggagcgcagtggtgccatcacgactcactgcaaccttcacgtcccgggctcaagtgatcctcctgcttccgccttccgagtagcgtgtggggagggtgcacgcagctgggctaggatggcctggctggggtcagcccccgtctgtcctggaggccaggcaggtgcggagcttacggaggtggccacctagatgatggtgcctactctcgtgagttgaggcccttggatctctggcagtcagcaggactgtggtggcagccggtgtgctggttgtcagctgatgtccctgtctgtgatttttccgtgtgaatgcagccttgactgggcatgtagatcagaggtgacaagtcctgggcacctctgccactcttcttcctgccctgcacccactacagacacagctaattggtggctgcgttgagaatcttgtcaccaggcaggctcttctgtcggctcaggactgtcctgcgggcatagcaggaagtgaactgcagggggaagttggggcctggttgctgaggagggcagtgcgtggtctccaagccagtcagtgcctgcgggggggactagaattctcacccaggcctctgctctcagtcctgttcctcctctcacattcttcacacccaggctgggctcagggatccttgttgatgatggccttgaaatgtcctctgaacagtgtggctctgggggtgtgtaaaacccagggtgatgcctgctctgaaaggggcttccagatccctttgtgtctccagcacctgccgtgtgcctgccccaagctggggtctgtgagctgtgccttggggcccctgcctgtccccgctctgctctctgctgtggcttgctggctgcagggttctccagtgagtgagggatccctgctggccatgcctggagctgagggtggagggaggcccagcaggaagccccacaggtaggccccagtgagggtgcattatctacaggaaagttgcctgggacactctagtgggccgtctaggaaatgcagcctttaaggacctgaaatggagggggatatttttagtttttaaaatttatttctcattaaaattataacacagcctcgtaaaaatattaaaacagcgagacagatgtgtcaggggttatgtatatgaaggaggtagcacagttctcatcacgggtgtgcttagtgtccccctaaagttcatgccagttaatgtgatcgtgactgtaattggaaatagggtcgttgcagatataattaagatgaggtccttctggagtcgagtgggactggtgtccttagaggagaagagacacggacacacagaatgccacgtgggacagaggcctgctgtctacacctgtgcagaggagacaacaaggagagtggggcatgacccctgcccacgacactcgcctccaggaaggcagccagctgcccatctttggtgcggtcatctctgcaggcaacagaatggcagagtggcagtctgatcactctactccattgtgtgagagtctccaggatgaagaccaggctccccagcaggacgtgcggccctcatggagttctggccttgttttcggctgttgcctggctgatattctgcacatctgcccaccgtcctctgggcggtccacgaatacattgtgccttcctctttgcatatgctggtccctctgctggcaatgcccttccttcttggtgagctccaatgcatccctcaagactcagttcagatgcgcctctctggactcatccccaactcccagacggggttggctgctcccttttttcagatcctgaaacacttacttgtccccctttatgtgggcatttatctgatagtagagtttttctgtactgttggaatggtgagttccctgaagctaggtcagcttccttctcagcattcagcaggcggtgggggaggagggaaggagaggaacttcctaataaagatgaacacctctttgtggttaggtcagaccagcctgtgcctgtgcgacctggtgcagaccttcacctctcagagcctcggtttcctcatctgagcctcaggcacgacaatctgctagactctgcttcccggtggcagctggggaatgggatgggaaggagccctgggatgctcaggtcatccctgccaccagaatgtccccagagtcatgaccatcagcacctcagcctcgctgctttttccttggggtctgcaggggcaggacgcaaaccccacacgctcgctgcgcagtaagctgcaggggcttctccagcagcacctctgaggcctgcctaccacgtggagatgctgctcccgtgggctctgaccaccgctctgccctcttctcacaggaaccagcgaccactcggcccggcgtaaccacgcatgtccactggtcacctggtggccttcgacaagaacgtgtgataaatccctgccggagtgcggctccgggaaaacgctgtcaccgcaaaccagctcctctttgttgcagtcccccgggggcatggcccggcaagtttccttagccaggggccagtttgcctggggtcagtgggcagcgcctgggagtcgagtgtctcgagtggggctctgcttggtttctccttgaaggtctggcagggtcagaccctgcatcggcagtaaccgcggagatgggagagccgagtctgccgctgtgggcttcctgggccttcccgccacccccgccagggcccttgggctgcatattcgaggcgcgcagcctgccaggccctccggcctccctgcgtggccaccagggaaaatggcttcactttgctgagcctctgtctccctggttacaaaacaaataattttattttttgtgacagggtgttattttttgtgacagggtgttgttctgctctgtcccccgggctggagtgcagtggtacgatcatagcccactgcagcctcgatctcctgagctcaaacgatcctcccacctcagcctcctgagtagctgggaccgcagccgtgcaccaccacgcccagctaacatttttatgtcttgtagagatggggtctcactgtgttgaccaggctggtctccaactcctggcctcaaaaattctcctgcctcagcctcccaaagtgctgggattacaggcgtgagccactatgcctggccaataatgttctattttaatttcctgtattccctaaatagtcataaccgtttccaagggcgatattcagaacacgtaatgagtgcagtgcaggcaccagcccacgtctcggccccccgtgtggggaaccagtctggccatctcatggtccctgcgtctgctctgcccacctcgtaggaggctctggattgaatgaggttcgacagtgctgactgtgagcctggccctgagtccgctcaggggtctcacttgccactctgctgctgtcactgcgccgttccctttgcccacatgccctcctggtctctgcatagatgtcctgagtgggactgctgggagtggggcactgctgcgtggcagtggagcctcgatagtgggtctcttgggggattattggcaagctgcaccccttgaaagttaaccgatcggggtgcatggcgtgaacttcccttcctgagctggtcagcccagtgtatgctgtgagccaggagccctgagggcgtctctgcccgcctatctcagagcacactctgtgttgcaggtgccgtgacacctattttacagcccaggaaacagagtctttcatgctgttccttccagccgaaccctcagctggagtcactccagaagccgcaggcaggagagttcccacaaaggactggatgcacaccttcctgagacggggggtgggttaggattccagcaaaggaagctctaaacaccagagtggccagtccatggcatttgttgggggacttacagagggggcttcagcttcctgtgggaatggggagatgagccacgttctgccccggtatgtctgcaacgacgggatcagattacggagttcatatgagggtgaaggaactcggctcagggccgggcccagtttccaggcttgatcttgcagtgtttgggggcaacaccctgaataaatgaatcagtcagtgcctgggaacattcgaggccctggcttgggttcaagcctgcaggaaaaacgtgcagctggcgggtcaggggcagttaggacagaggacgaggaggaacgggggcccctacgtgccatgggcatgtgctcgtctctctcctggtgtccgcctttctcctctgccttgtggctcagcttggactgccataacaaaatacgatagagtgggtggcagaaatgcattttctccccgttctgaaaactggacagtccaaatgcatggtgcctgtggtgttccgacaggggatcactcctcgggctccttctcgctctgttcacggggctttccttggtgcatggagaggatctccctctcctcctcttcttataaggtcaccagtcccatcagattagggccccacccatatggcctcatttaaccttaattacctccttaaaggccctatctccacatataggcccttgtcatagcggaggcttcacataggaacctgagggggactcagcctggagcacctggtgtgttaggaggaggccaggccagcgctgagcaccctgcctgtgggtgagccctgcttctcaagcaacagtaataaataaaaaggaggaggccaaactgaccagacctaaaaaaacagtatcttaaggtctgacacctgccacggcatgaatgagccagagaaagtgatgctgagtgaaggaagcctgtcacaaaaggacaaatgtggcatgtgaaatctctagaacagggagattcatggggacagggagtggactgggttaccaggggctggggtgggaacaacggggagtggcttcttcatgggtgtagggtttctgttgagggcgacgacagtaccctgcaagtggaccggtgaccgatgcacagcgttgaatgtactgaatgccactgaactgtgaactttaaaactgttaaagtggcaagttttatgttatattaaaaaaatcatatatagaaaggatgttgtattaaacgagaaaaactcatttctctcaagtaccagtcactcattgtggagactgctcttgaggtcactgtgcccaggcctcttctgtcttgagccagtccatctctggggtgtgaccctagcttacccccatgaccacaggaagaccatgagctggtcctttaaggatgcaacctgggagttgccccgaggactgtgctcacattctgttggccaaaactcggttacgtgaccacacctagctgcaagggaggctgggaaatgtagtccttagtctgggggcatgtgcctggctgacaatggggcttcagtgctgtgggagaggaggagctggtggctgccaggggatggacagctgtctctaccaacctggcctcacagtggtcacgctccaggacataaatgtcccctctgcagcaaagacttcctgggtgtggaggggactgggggcccacaccctggtcttcacaagatggtctcctaccccggaaggcaccatgcggcccacgcttcagtgtgaagacaccacctgtgtgttaatgggcccaacaccaacctgggtgcagggcaggtattcagcaaacagtgaatggacaccagggagctggctactatagcgacctcctgaggtcgcagggctaccaaacagtgcacggcatttgcaccccagctcaggcgtcccggcagggattctgcattgtctgcctttgaaaaaagggtgggagagttaggacaggaattttctctgtttccctctctctctccttctccccgcttctccccctctctttttccctttctcttttcctccctctctgtccctccctctcttttcctctctatctccctatctgctttgctctctgtctctctgtcctatttttctccatcactctttgtccttgtctctttttctcgttctctgtcactccgtttctgcttttgttcccccttctctctctctccccaacccccctgtttctctatctcttcacctgtgaattcctgaaacagacccagggcatgatgtcgtggagggcaggccccagaggcgtccaggtgacaccgtgtgggcatttgtaaagcaggaggccccagcaggtggagcaggaggactcacccccctgcagtgggtggtcaagaagagctgcctttcctaagcccctctctcctccagccacccctcacctggggcctgctgagagggacaaggtggattggggcttgctgtggggctggtgctgaggcaggggtgggtggccccccagcccctatttcctcctctccaaacccagccatcccagttaattatttgcccagcagggcagcttgactggctggtgtcttgctgagcaataagcagctgaataagggtgcaattgctggaggcggggtcgcagctgcagacctgggcggtcacttagtctgggacagctgcggcggccacagcgaaagccatgcagcctgccccatgcctggctttgtcgcgacggctgctggaacagacgggtgtggtggctcatgcttgtaatctcagccctttgggaggccaaagcaggcggatcacttgaggtcaggagttagagaccagcctggccaacatggcaagatcccctctctactgaaaatacaaaaaaattcagccgggtatggtggcaggcacctgtaatcccagctactcaggaagctgaggcatgagaatcacttgaacccaggaggcggaggttgcactgagccaagatcaagccactgcactccagcctgggtgacagagtaagactgtctcaaaaaaaaaaaagaaaaaaaaaaagaaaactacacacacatacacacaacataaaatgtatgattttggccaggcacagtggctcacgcctataatcccagcactgggaggttgaggtgggtgaatcacttgaggtcaggagttctggaccagtctgggccaacatggtgaaattccatctctactaaaaatacaaaaattagtagtgtattagtgggtgtggtgacaggtgcctgtaatcccagttactcaggaggttgaggcaggagaattgcttgaacgcaggagacgaaggttgcagtaagccgagatagcaccactgcactccagcctgggcaacaaagcaagactccatttcaaaaaaaaaaaggatgatttttaaccatttttaagtgtaccgtaagtggcattaagcatgtggtgcaaccatgaccaccatctatctccagaactcctttcaccttgcagaaatgacaccctggacccttgaaacactaactccccctcccaggttccctcccccagcctggggattaggcatctagcagctgctgtctggagagggatagcatggccctgctccgtcccttctgggcccgttgagctctgctgtgaaggggattcctttccttgtccctgtcactcctagtgtggaggtcgggggtaccaagccccagccctgccacctggaaaccctgggaaagcagcgtcacctccctgtgcctcagtttcctcatctgtaaaggggagctgaggacaggacctgcctccgagcctggggagagacggcagtgcacagggactagctgtgcgtctcagtaatacggagagaactttgcaggccaatggcgtccctctgagaaagactctccctggattctgtttcagagagttgagcttttccccagagcagatgctttgaaactgtctcaggtggacagagggggctgcagggagggcacagaggacttcctgggagtgaggtttcctgggattgagtcccagccctgctgtagggctggagtgaaaggggcctccccagagagcctctcccagggctctgtccccaagtggccaagctaattcggttccgtgctccccttttgcccacccccaccagaggccacaggctggactgggcgccactgctcatgcttctgtcctgggatagcacagaccagtacaccagggtctggcacatgtccgttgtagtcactgcagccctcccagtctccccgttttgtagaggaagccacagctcggggcaaagccgcacctcgcagtggacaggtgagtctctgcctccccaccagctgcctcttccacagatggacccccaatcacactgtcatatcgcctgtctgagcctcactgtgctagtctgtaggagggggcacctcctcttgggccatccatataaggctggagacaaacagctccacccagttatgggagtcccatgccatccaggacacatccaagtggatctctcatagcctatcatggttcctccaagactcagttttctcatctgtccagtggaatgacaatggtacaggctgtgagaatcccttatctgaaatgactgggatcagaagtgtttcggatcttggctttaagttgtagaatatttgtatgcacataatgagatatcgtgaggatgggacccaagtctaaacacgaaatttataatacataagtttctggaggctccagggagaatgtgttccttgcctttcccagcttccagaggccgcctgcgttccttggctcgaggtccctcctccaccttcagagccagcggggcagcctctcctctcctctccgacctcctgcctccctcttacaaggacccttgtggttcggggtcatctgattatccacccagataatccagggtcatctctctctttcctgatccttaactcagtcatatctgcaaagtcccttttgccacataaggggacacattcacagacgggttctggggatcaaaacagggacatctttgagggctgtgtttctgctgaccacgctctcctctgggcctgccctttgcatctgcagatagcagtggactctctttagggggagcaaagcctcccgctctgagcatgggctggactttgggggtccaggactggggaggggtcgacattgtaggtcaccaccatgtgtgactgtccagggaggctgggggatgaaacagccgagggtctgactgggggagagccgagaacacaccccactttatccggaaggccagccatgtctgctcagagacgaagagtcacgggacagccctgtccaagagcaaagacccgtccaagagcagggtcagcacctcggagagttccccgcaggccgccacggggcggggagagaattcatggggcttcgacttagaaggggaacatacaatgctgtggttttcactgcctctgggtgcaactcagcatttccccaattacgaagccagggccttggcccccagcaggaagcctggggtatttcattgcctcacactcacgtcccatctgctgaggggcacgtacgccaccactactcctgagtggcagttgattttacgctcacagattcgtcctgttatttagtatgttaattcaggcggctatgcatggttgtctcacaaatttgatttttaaggaatttaacatttcaaaatacttggtttcccttgtaacccctcaccttttattttctgtacctacggtcatccctcaagagggatccgtaggcctcccctgcctgccctgggcacagggacatcccgaagcgccggtcccagagcctgggctctgctgctgtttctctgtgacttgaggcaagatatcctacgctgccgagatagcagcccctctgcctggggaggtgagtgtgaggaacccggtgtactctgtcgatgtttaagggtcagtgggcgccgaatgaatggtttctgtcagcagcacattctgcatctcccaaaaccaaaaccaaaaggacagccggattttcccctctgcctgacttcactaggctcgcgtctgattttttttttttagcaccgtcagcataagggcctgggtttattttgtggttgtttatagtggaattgggtgtagtgtcctgatttgttttccaggatgttcgttttggggtcaagggtctttggggtggatgaggcgggagaaccaggcttgagtgaggctctgccctcctttcctggccccctggtggcccctccagagtctgtcccggccatggaccccagtccccgctgagccatgcggagagtttgggagaagtgcatacctgccttgggggcttgactggcaggctgctttcctctggctcatcttgaagttaaaatgtagctcagaccctcctcagcccccacgcagggttgcaacctcttccgtctgtctccctgtgaaatccaccttccagggggcagcctgggacctgggatggccctccctacctgaaacctttcaggccttccactgcctgcagggaaaagcaccggctcctttcacggacttccagcaccccttcggggctgctgctgcctggttggtaagccctgagctcactaatctcagccgtcacatgctgttgggcaccagcctgtctctttggaccataaaaacgccatgttgggttggcaggataagtcggcaagttttgatccagggctacagatttcctggttgtattgtttcaaggttaatttctttctttttttttttgagatggagtcgcgccctgtcacccaggctggagtgcagtggcacaatctcggctcactgcaacttccgcctcccaggttcaagcaattctcctgcctcagcctcccaagtagttgggactacaggcctgtgccacgtgtccagctaatttttgtatctttagtagagacaaggcttcaccacgttggtcaggctggtctcaaactcctgacctcaagtgctccacctgccttggcctcccaaactactgggattataggcatgtgccactgtgcctggccaaggttaatttcttgatagagttggacagtcggttttgctacaaagcttgttttgaaaatgagagttggttccagcatgattgatatatcagggaacagtttcaagataacacaagtttcccgttttcctttgtgagatttcacccaccagaagcactgggtgaacacagccactgcccccagctggcctgatgcacctgtgttcacataggaacacataggatgcatacgcacctcacacagccccagccgccccagggaccccatcctcgtcagggcacaacttccccgtctccactgccccgtctctgccactctgcaccagctctccagcccacctccctcggtaaacttacagcgttttcaagcccaagtgccgtgttaatgtgtctctgaaccccttgacatgtgaagctgtgctgccgtttttattagggtttctgtctgttttgagtgccctttccctaaccccattccccaacaagccctgtggttttgactgtgcggttttgcatgcagtgatttctaggaatgcagacgtcgagttgtggtggaatcgactcttctgtgctcatgtgagggaatggccttgttttgaggaaatatgcgctgaagtactcaggggttaaaaatcatatggccagctgggcgcggtggctcacgcctgtaatcccagcactttgggaggccgaggcgggtggatcacgaggtcagcagattgatagcatcctggctaacacggtgaaacctcgtctctactaaaaatataaaaaattagctgggcgtggtggaaggcgcctgtagtcccagctacatgggaggctgaggcaggagaatggcgtgaacccgggaggcggagcttgcagtgagccgagattgcgccactgcacctccagcctgggcgacagaatgagactctgtctgaaaaaaaaaaaaaatcatatggcccgtcaggcgcggtggctcacgcagtttgggaggctgaggtgggtggatcacttgaggtcaggagttcgagaccagcctggccaacatggtgaaaccctgtctctactaaagatacaaaaattagccgggcgtggtggcatgcgcctgtaatcccagctactgaggagactgaggcaggagaattgcttgaacctgggaggcggaggttgcagtgagccgagatcgcgccattgcactccagcctgggcaacaagagtgaaactccgtcgcaaaaaatagtaataattcgtccgtctcctcggcgagtggaggctggggccagggtggaacaggagctccacggtgagccaccccggagtgaaggccactggccctccttatccttgctgcggtgtgggggtgtgggtgtatgacccacagctcacatgtggaaacatggaggccccggggcctgtgtgacttgcccagggtcccagctagcaggtggcagagcaagacctgtatgcagaggcccaaggaagcgtcatgaggacccaggtcaccagtgcatctccagggtgaacctggcccctgcccagcgcatttgcctgtcccctgaaaccgccaccacacaaaaccatgtgcatcttcagagcaggaggcgtctccaccatctgtttgctcccgtttgacaaatggtcggtgctccttgaacggataaacaggaaccatccggccctcttccttgccggagctcatccctttgactccagggagggaggtcatgagcaccacagtctttatgggggagatcactggcctgcccctgggcggagaagccaaactagaaccttctccccagggtgagctcacaggcttttcccatccgggacccctgagccgattggctgcccgggacctgctgttatatcgggggctcccattgcaaagcccctgcgtgggctggcttccccgggtttggtttctccccattgtgcagggtcttggggtagggtctgagccaggtcaccctggagaaccgacagcaccggcccagccccgctgtgacctgctgaccccggaccccgcactttgcttagtgctttaggtctgtaggagtaatctttggagaatcgtgattctgggacccgagctctgagggaacaacctcctctgagcctcccaaggccccccaacattgtggggtgagccccacctcacagatgctgagacaggcctagaggcgtggcgctcccagccccatgccccacatagctctaaggcctggcctcacttgcaggtgagggtcctaggaagctgggggtgggagcaggcctaggagaggcactcaacaaccacggctcagcatggtctagaactgccccctcttcctcccggctgccccgagtctggcttccgtcagacccagcctccttccccagcccatccctctccctctttgatgaggccgatggggcaaagacctttgcttcccagaatgttctgtccctggttggaacactggtccctgggacacagccctgtcctcagctttgaagtcagttcccatcgctgccccccaggtagctggctggcttctgccaacctcagcctggtcgcagctggcacctcctcaccaggtactaaaccagggtgtgcagaggcccgctcgatccatggggcagatcagcaaatggtagtgcaggatgccccatcatatgtgaatatcaggtaaataacaggtacttctttttttctcttttttttttttttgagacagagtttcgctcattgcccaggctggagtgcagcggcatggtcttggttcactgtagcctctgcctccctgatttaagcagttctcctgcctcaggctcccaagtatctgggattacaggcacctgccaccatgcccagctaatttttatatttttagtagagacagggtttcatcatgttggtcaggctggtctagaactcctgacctcaggtgatccgctcccctcgacctctcaaagtgcttatattacaggcatgagccaccgcgcctggcctatttttttttttttcccttggcaacagggtcttgctcttttgcccaggctggagtacagtggcgcagtcatggctcacttcagcctcaaattcctgggctcaagtgagcctctcaccttagcctcctaagcagctgcaactacaggtgtgcaccaccatgccttactaattttctttaaaatttttttgtagagataggggtctccctatgttggtcaggctggtctcaaactcctgggctcaagccagagaggggctggcctggtgctctgggttctccagaccccctcctgccttggtctcccacagtgctgggattacaggatgagccacggtgcatggccacctgatggataatgtctaatataagagaacccctaatttagctgtccctgtacccttgtgaggtgagcagtattgttgcctacattctatagatggggaaacagaggccacagaggcctagcagcctgcctcgtcaccgagtgggagcttggcagggctgggatctgaacccaggccgtcggctcctgggcctgtgtgccccctccatgaccatatcagcttcctacagcttccatgacaaagcaccacaaaaccggaggcttaaacacagaaacgcagtcccccagctccgggcagaagctcgagacgagggtgtggcagggccgtgccccctctgcagcctctggggagggtcccttccggcttctgggggctgccggcctccctggagtggccacgtcacttgttgctgctgtctccctcgtcacgtggcctctccttctctgtctcttagctccctctgccccgtcctgagcacagctgtgattgcattagggcccacctgtataaccgaggatgccctcttctcaagagccgtaccttgatctcatctgcagagatgcttttcccgacgtgaatggaatattctgaggattaggacgtggacctaccttgttaggggccaccattcagctgactgtctttgccaagcacaggcagtggagactgtgtcatgctctgatccgaacaatcgtccttctgtctgccgcccgacttccccagccctgttcagaaagacagaaggaactcctgccccaccctaagctgaagtcgggagcgtgaggaccctttatctggtctttgggggccaggtgggttcacgggcacaggacagcatgacaggcagggcctggcattgggacacctgggtcttagtcctcacctgtgacagcggggggtcttgggcaacgtcccacccctctctgggccttgaagatgaaggggttagatcctggcctcccggagcttcccctggtgtaggatcaacacactttttctgtcaaggactagagagtattttaggctttgctggccctgtagtctaagtcacatactcttctctgtctgtttttgtttgttttgttttgttttgttttgtttttgtttttcctacaacccctttaaaatgcaaaacccgttcctagctccaggccatggctgccattgggctgtgttgagcctgtggcgcgtgcacaggcccctgcccttggtggacactctgattccacctgcaaatcctgagcgcacaccggctctgaacaggctctgtggcttcagaaacttatcttctggcttcgtcgatctggctggtttgccctggggatcccgcatcctctgactaccgtgatgagcagagcgcagcgcccggctccgtgggccaacttttggtgtcttgccctgcctgagcctgttcctccttcctctccctgcccctccagccgctccgggtcctctgggaagctctggcctcttcaccagccctgcgcggctgctggatctggaggccggagccggcacatgcgtctgacagcagcgctgctgtctcttgtttttcgtggtccatgtttgggggttacatctgggcctgtcccttctctgtgtagcctgaaggcagttcccaggcttgtgtccctcacctctccgcagacgccctttccagatcctgttcaaatcctggccctactcacgggaacttgggtgagtcagttcccttcactgagccttcatgcctccctgttaaggagaagcttcactcggcatggcgtggcccgtcacactcatcctgcatctgttacaccaaatgcagtttcctcttctataaatgggagaaaatcacctgtccttgccaagggtccggtgggaattaaatgagctgagccagggcacgggtggagcttgctgagtgttaggtcgtgtgcctgctcttcttggcggagtctggccagatacctcatctggttccattgctgtcagtctctaaatggggcctttcctgcccagctcccagccctggtgctcacaccatcacaggccaaagaactggattccagacactgcctcctccactgtccccacagtgaaaatggggagggatgtccctgtggagccctaactgggacccagacagccctacccccagctcccaccccctccggctgtcccggaggccgtgctccgtggatctcctctccctcgcctccacgtgggctccaggtagctgcacacgcattacaaacatctcgttaatcttctcggaggtcctgcgaggcgaggcctgtggtagccctgcccttggagcactgagaattagtaaggaaatcgccgctgcatctcccggtggcacaggggcgtggaggcgtggcgggccttccctcttgcaccactttggatcctgggtcctcacagcagtgttctctgcaggggaggtgcaggcattggtctgctctagggccaggtggtctaaaaagtccctggtcagccccgagtcctgcaggaatctgcatgttagtgggatttggggttcaggatgcttgcctgctgggtaggatggggttgctgagtgaggctgaaatcccagatgggatgtctgggctcctccatgcctgacttgggaggggtgagaaaaggaaggagacttggccatctttaggctaaaagcctagtgctgtctcacaggagggaagagcaggtctctgcaacacacgcctggggttccgtcccctctggccacttgtctgtcaccttggacacgttgcctggcagcctctgagcctcagtgtgcttctgtgaattggggtgaccctagcacggtgctcacgacagaaagcatgggagtgcagacgtgggtcagtggcccgcggtgagagctccctgtctgccccatggtgagtttccacagcagtgagagaggcctggcgtgtggggtggaggccgggacagtgtgagggccgcctggtcagagcgggaggcaggaccccgtagggagatgaaagccaagctggacccaggcagaaaaagaggaacgaaggacaggaagtccaagagcagtcactgctgacgtgagtgcggcctgcaggcagccagtgtggatggactctcaggccctccgttgtgtgttaggggaccggaggagcagagaggttgcagcagccggccggcctctctcggtgagggccggactgcacatggggctcagaggccgtgggtgtgcctgcccgcctgtgtcagctccagccgtgggggcgggggtaatccgaggctgcgtcccgaggctgtgggagctctgccttcatccttccattcacaggtagggacacaggcgtggggattccacggtgcccttgaggcagctgtttcctagacccccaaggccctgacggagactcccccacccctcaggagcagagggtgaggcggggtctgcaccggccgatgtgggcaactgttgcaccctcggagcccagcagcgagggtgggggtctgtggggagtcagccgcagccaccagcattgctggaatgtgtcagaatggtggtgcctcctgcaaacaaccttagcctctgttcatcctgtcaccagggtcctgggagaagcaaacccacgtgtggcctctgcagggtgggtggggaggttctcccggccaggcctgactgacgcccctgctgctccctgtggtccagcctggacttgccaccgattattctatcaatggcactgcactgggcaccaggatccaagcagtggactgcgggacaggacctgtccctgtgagcggcctgtccctctccccaggcccagcctcctgttggttatccttccaagcccatgcctgggctgtgcgatccacctttcctccaggcactcttcaggcaaatctctggagcacccactgggtgccacacactgcggatgggacgcagggtaccgcacagccccggtcctcagggacccggcggggacagcactggtgaaaaaccacggaagtacattgatgtgtcacttcaacaggggcctacgcaggggcctgccctgtgccaggccctgtcgttggtttgtggggtgaagaccctgttcctatggtgtggacgtggtagagatccaggggctctggcagcttgtatgggggaatggggacgggaggggggtcaggaatgggcttctcgagagaagtgacatttggtcagaggcccaagagcgggaattgatcctggccctccctgaatcagtgtggtgtgacccctcccgccaaggtgagggtagcaaggtgcctggggctggaatcctggggctgtggcccgtacagtcgcccgggacccatgcccagcagggccctgcacccggagtttaacactgaggtccccaccttgaaattcctaatcattgagtctttgtaacttgtgttagggagtccagcgagacaatggggtatgtgcggggtctttggagcttcagcccatgcacagacacgtctctctgttcctcgactcctagcatgctctcacctgcctgccccagagaccaagccccacctggccccttcgtcaccccgaggtggcgactgggttgtgctgaaggagggaggcccacacgccaccactgccctctgacccgtcaggggccccgagggcctggttggtcagggctgggcactccacccaccccggatgacagcaccaggctcatgtgccaggtgactcagccagggccactcaccagcgccacctggtatgtcctgggtggcggccgctgtgcctgctcctatccaccatggttgggcactggctgggaagagaggtgaccaccaggccctaatagcaggagtcacccacacaacccttgtacccagccctggggtccaggccgcctgcaccggctcagttctgcgcctgagctcccagctctggggtccaggccacctgcaccggctcagccctgcacccgggctcccagtgctggggtcccccaccccttggtgttgccgcatcctccctgggcccaagattcctggagctggagaacccccttcctccctgaggcatggagactgggctggtcctgagcccactccaggcacaggggacacacctgcttccttcttaactgagtagagcagagttattttgaagatcggttttcagcaggacagtaaaaaggcgaactgggagattgttcatgcagcagcttctaaaaccagtttttagataaatgcaccttcgtctgtggggggtggccgcacccagcctcgcttcaggtgcctgacggggtacgtgctgcgttcagtcccaggcaggtgtagggctgggcaggtggagatgcggtgccctcgctcaaccggggtctaggctcctcgccgggcggggttcacctgctatccccagtgtgtgggggcgggggtgtggggtgagggagaagagtggggcactgagtctaaacataggctgtggcctgtaggtgaggggaggcggcagcaaggagggggagatcaactcctgaagagactccgtttctaacacgccgggagccctgcgctttgtcatggaaactggcaagagtccctctccatccctccctactcccagaaacaagatggtcccaggctaacgggcttctctctggcctggccctgctcccagacccccgctgagggtggtaaacaccccagccgcgcccttggcaggggttccgtcccaggggcctccctgttctcactgtgagggtgggcatgatgggcagctttgcggagcctcctcagcacagagccgcacatgacgggggtggacagactgtggccccacagccgtccatcactttcatcctgtattgggggagaacggggcttgcttgaggtctgtcctggatattcgtaatgctggcattgacggggccagtgaaggctctgattggtgcgggtgacgaggacacagtcgcagcaggcgtagaccaccagagcagggtgtccacgtggctggccacgtttagttgataaaagtggcatctataaatgggtggtggtgcggaatgggtggttgattaaatggtattgggccgctcaggtggccattggggaaaggtaaagctggatccctacctcataccaaaattattctaggtggataaaaagtttaaatgtaggccaggcagtggcttgtgcctataatctcagcactttgggaggccgacatgggcacatcacctaaggtcaggagttcaagaccagcttggccaacatggtgaaatgccatctctactaaaaatacaaaaaattacctttgcgtggtagcacgtgcacgtaatcccggctacttgggaggttgaggcaggagaatcgcttaaacccaggaggcggaggttgcagtgggccgagatcgcaccgttgcactccagactgggcgacacagtgcgactctgtcattaaaaaaaaaaaaagtttaaatgtaaaacataataaaagctggtttccttactgcataaaacacttctacaaatcgacaagaaaaaaaaatcaaccgcactcttaataacagaaatgtaaattaaaactacaaggagataatgctgggtttcacttgtcttgggagagatttaaaaaaaaaaactgatctactgaattacagaagatgcgaggaaacagaccccctcgtgctttgctcgtgggggtgtaaatgtcctccctggagagaaaattgtgaatctctagcaaagttcagaccccaggaacatttaaactgcagctttaattcaacagcggcacttctggaagtatcttctgaagatggatttgtgtgtgaaatatcaaatatacaaggttgttattagactgtgaattctaataggagaacatggaagacagccttcatgtccatcagcatgggcggacttcatgtgttctggaacggtcattaaaacaagaggctgggcacggtggctcacacctgtaatcccagtactttgggaggctgaggcgagaggatcacctgaggtcaggagttcgagaccagcctgaccaacatggcgaaaccctgtctctactaaaaatataaaaatgagctgggtgtgatggcgggcacctgtaatcccagctacttgggaggctgaggcagaagaatcgcttgaacctgggaggcagaggttgcagtgagctgagattgtgccactgtactccagtctgggtgacacagcgagactctgtctcaaaaaaataaaatataaataaataatttaaaaattatttaataaataaatagaataagaaagctgtgtttgctgatatggaacaatctccaagatacagtgttaagttaaaaaaaaaaacaaaaacaggccaggcgcagtggctcacgcctgtaatcccaacactttcggaggccgaggcaggtggatcacgaggtcaggagatcgagaccatcctggctaacacggtgaaaccccgtctctactaaaaatacaaaaaattagccaggcgtggtggcgggcgcctgtagtcccagctactcgggagtgtgaggcaggagaatggcgtgaacccgggaggtggagcttgcagtgagccgagatcgcgccactgactccagcctggacgacagagcgagactccgtctcaaaaaaaaacaaaaacaaaaaaaagcaggggcagaactgtgtgtggtgggccaacacttgtgtccaggtttctagcatgtgagagtgtttgaaaactggagcctgtcctgtgacggagtcttgtgcagacatgaaaaacaagagatatcctagtgagctcccaaacagggacacccatgacagagtaaaagcagaaacaagtcacaggaaaaaatatgtagagcataaccccatttgtgcggcaaaaataaggtgtgtatgtcgatcactgcatgggagaggaagcgaaagagaaccactcagaatgaagagtcattttctgtgggaagctggacacatgtggaagggtttgaaggaggagttttactttcattctgtacattgtaaaattttttctaaagaacttgtgttttacttttgtatatttttatttatttatttattttgagatggagtcttgctctgtcgcccaggctggaatgcagtggcgcgatcttgtctcgctgcaacctctgcctcttgggttcaagccattctcctgcctcagcctcctgagtagctgggactacaggcgcctgccaccatgcccggctaatttttgtatttttagtagagatgggggtttcaccatgttggccaggctggtcttgaactcctgacctcaggtgatccaccagcctcggcctcccaaagtgctgggattacacgtgtgagccacagcgcctggccaacttttgtatattttaaaaataaaatatccagccacaatccctgagaatagctaggcagtggaaggatgatttctaccaccccatgccccaccccccagctggcagaagctttcaccccccgcccctcccccgccgtttccccgactcgctgacccctcctctgcccctgggccttttgcatcctggagctctcttccccttgccaactcctcctgcctcccaggcctcagccacgcctgcttcctttgggctgatgctgagcaccaacccagagtgtcggtgctgctcattgctccagtgaccccgtccagaccagcctccccgtccagccttcctcttgctcctgggcaggacctgccctgtgctttgaggccctggggcctgcacatatccagcatgtgggtgtgccctgtctgtgtgtaccaggtaagcgggcagatgagcccagcagacactgctgcagccggaggctgccggcagcccgcagtggcacagccgtgctgtgggagggcctttctgtgtctggggctgtgttttccacactccccctctggcttttactgttttgctccgggagtgtggctttctagagttttgtctgattttatagcagagtttctgttaactcaaaaagggactccacagtgaaggtttaaaggtggtcccagctgcagcaggaagcaggtgtggggtgcctgctgcccaggccctgttccccagcactgcttctgacccaggaggggctggggagaggaccactattcctcacctttcatacacccactcccatcctatttcgcaccccttcctccaggctgcctgcacctctgccctgctggcctttcgctctgtaattcctttctgaaagaaatgttttccatgaaacaacaacagcaagaagaaagcagatcagctgttggcaggagctgggaggagggatggggagggcagctgatggatatggggtttctgtcgcaggcgacgaaagtgctgtaaagttagatcacgatggtggtgacccgcctcagtaaatgtgctaaaaaccagtgaattatactctcaacaggagtgtaattgctgtcttcttagaacgtgtggccatggaaatgtgctctttttatgtgtttacctttgtatcagccgtcttctcaatgagaccagaatccctatcagagcaaggaccttgtttgtgtctcccttttcctctatgtcctaatatacaacagtgtgtttagcttgtagcagacactcaatttaacaacatctttttgaacgagtggatccataaatgcattaagtggctctgtgacttcagactcttcttcaggtttacggtatataaatatatcatttacggtaccttcatttatggtatataggtgatattttagccggggtgcagtggttcacacctgtaatctcagcaccttgggaggcagacgcaggtggatcacgaggtcaagagatcgagatcatcttggccaacgtggtgaaaccccgtctctcctaaaaatacaaaaattagtcaggcatggtggtgcgcgcctgtaatcccagctacatgggtgcctgaggcagcagaatcccttgaacctgggaggcggagctcgcagtgagctgagatcacaccactgtactccagcttgggtgacagagcaagactccatctcaaaaaaaaaattgttatctcagccaggcacagtggctcacacctgtaatctcagcactttgggaggctgaggcaagtggatcacatgaggtcaggagttcaagtccagcctgaccaacatggcgaaaccccgtctctactaaaaatacaaaaattaggccaggcgcggtggcgcacgcctgtaatcccagcacttggggaggctgaagtgggcggatcacgaggtcaggagatcgaaaccatggtgaaaccccgtctctactaagaatacaaaaaattagccgggcgtggtggcgggcgcctgtagtcccagctactcgggaggctgaggcaggagaatggcgtgaacccgggaggtggagcttacagtgagctgagatcgtgccactgcaatccagcctaggcgacagagtgggactccattatacacacacacacgcactatatatatatacacacacacacacacacacacacacacacacacacacacaaaaaaaaaaattagccgggtgtggtgggcgcctgtaatcccagccacgtgggaggctgaggcagaagaattgcctgaacctgggaggcagagtttgcagtgagccgtgatcatgccactgtactgcagcctgggtgacagagcaaaattctgtctcaaaaaacaaacaaaataaatgatatctcaataaagctgctaaaaaaaactcgcccaccaggtacccatcaggtatcttatccagcataagaacagctgggctgtgtgctgtctgcacctcatcccatgaatctttgcaggggttctacaagggtcctccttactaaaccccctgcacaggtgaggaaactgaggcccagagattatgcagcctgcccaggtaaagcagctggtgaacgtggacagcgtggcccccgtcataccttctcgccagctgacggtgctgtccagggagtctggaaagcagagatccaaaggaattggctgggctggaaggctccttacagaagagctcgcttgcccattcactcattcatttgttcagctgaacacccacctacgagtgtgtaccaggcagacccgctcctgtctccacgaaccacaatcacagaattgtgtaaacgaggtaaagccggaggcctgagattgtggggatggggattgaggtcaggactgtcttaccaaggagtgacatttggcctgggattgaaaagtcacgaaggaactgaacattaagaccctgtatgtaaggctgggtgtggtggctcccactgtaatcccagcactttgggaggccaaggtggaggaatgcttgaggccatgagttcggaatcagcctgggcaacatagtgagaccccatctctacaaaaataaacaaaattagccaggcgtagtgtcttcctgcccacttgggtggctgaagtggaaggactgcttgagcccaggagttcaagactgtggtgagcagtgattgtgccactgcactccagcctgggtgacaaagggagaccctgtctctaaaaaataagattataaaccagccaggcgtggtggctcacatctgtaatcccagcactttgggaggctgaggcggttggatcacctgagggttttgagaccagcctggccaacatgacaaaaccctgtctctactaaaagtagaaaaattagccgggcttggtggcacatacctgtagtcccagctacttgggaggctgaggaaggagaatttcttgaacctgtgagaaagaggttgcagtgagccgagatcatgccactgcactccagtgtggacagcagagccagactccatcacacacacacacacaatatatatatatgtgtgtgtctgtgtatacacactcacatactcacactctctgtagtgggctgaatggtgtccccaaaagctaggtctgcctggcaactcacttccaacttcttggaataagggtctctgcaaatggaattagttaaggatctgcagatgagattctcctggatcaggctgggccccaatccaacgaagagtgccctcgtgaggcagaagaggagagagacacgtgccggagaaggccacatgagggcagaggccgagaggcgggagtaaggccaccacgagcctgggaacgccgagggcgccagcagccgccagcagctggagagaggatggatgggaggattttctaaagccttccgataggggtgtgacttcttgattttggactccgggactgcaggaccgtgagaaaaggagtgagtgttaagtccacctagttagtggtcattcattaccgtagcccgggacacttacgtgaacttcagacctcaaatcctccccatcttgcagccccaggcgcagacctgggcagtggggcccccgtgtgcccatcccccagctggccaggctcagctgagagctcctcgacctcctgcctgtccctgccacccttaggagtccgagaaactggacctgcccgtggcctggacaccacttcctttattgcttagggtttctctctttctttctttaaaaattttttaaatgaaaaatggggctgagggaagactcggtctaaagataagagaggggtcacctggatctggaatgttctctggctttccaggctgctctgatcgcagacctcctgtctttaacaacccagcctgggtcaggtctgatgaaatgcactttccagggaatggggctggtttgggctctgcagaagcttccctcttccatcctggcaccggcagaaaagcccattaaaataattgcagggagagactgttacctaagtttcctccccttgagctgttggagctcttctttgacctttatggtttttaaaaccctgccaatccttgccttgtcccccggacgcattcatccagtgggttgggaagctggtgttcgctgtccgggctgggtcgtgccggctctccgtgtgtgcacgtgtgggtctgggctgggtgttgacctgctcctgggccagggcccaccagagccttagtccgggtggccaccatctgggaggcccggccgtgtcctctcttaccccagttctccaggcactggccaggggccccctcagcacacatgtcatagttggtccctccctgcccatggcccttcagaggcccccagctggtcttgggaggagggtgcctgtgtgggccagctttcagcacagcccccgccctctgctccagccttgggtgcaccccaaccccacccagctgaccagcctgttggggggcctgtggaccccgcctctaagcctgggtcttcctctgggcacccgttggggacagggcagccacacacccagcccagggtgtggtctccccacccagctgcctcccttgcaaaggcagggttgccttggcaatgacacctgaggaggagacgcttgccctgggtgcgctgtttccctgtcttcagtaagggaggaggctggcttcctctggcggagggaggcagcagctctcctttgccttggaacccgctttgctccagtgatccctgagccaccacattggtaagactggctggagctgaaggtccgagtgtggcttcaggactggtgacgatggccagggatgaagggctgcggcgtgtggaggggctgcgcccggaaggaagcggtgtggacccagaaggaagcggagccctggtgggcagcctggctgtgctctggcccaggaaccgggccttttcttgcctcttgcctctgactcagagcggcttgtgggagcctcgcatgcagggccaggcagggcaagaacgatcgcgaccttttcctccagggaaggttgcaagaaaggcgggatctgtaacgtgactaggtggaggcactacggagccatggtgcaggggtggggggccccaggtgagctcagatgccctccctgcctgtgccaccgtggcctgaccctccacaggtgggagccataggcgtcaggagccattaccccttggagaactgggtgccaggccgagacctggtgtctcatttatgacaaaccgctgcagtgcgtttgggagtctcctcctccatgacgtgcttgtctggattggttttattttagtgcaggcaccacgtcgtctttttatatacgtgtgaacgtatatatgtaaacacacgtatgatgttggtgagttcatgttggtggtatttttactaagcaaagatgtgggaaccaaatgggcccatccggggagtttcccatttggtccctcgtgctgtgtgtggtggggggcattgtggtcgaggtctgaacagccggggaagtgaaagaccccgtcctctccgggagcttacgggagtgggagagacattgacgaaatattacccatcgcaggagtcaggtgcacacagtgatgatagttccgggaggtcaggtgcgcgcggtgatgacagttccggaaggtcaggtgcacgcggtgatgacagttccgggaggtcaggtgcacgcggtgatgacagttccgggaggtcaggtgcgcgcggtgatgacagttccagaaggtcacgtgcgcgcggtcgtgacagttccagaaggtcaggtgcgcacggtgatgacagttccgggaggtcaggtgcgcgcggtcgtgacagttccgggaggtcaggtgcgcgcggtggtgacagttccgggaggtcaggtgcgcgcggtcgtgacagtgccgggaggtcaggtgcgcgcggtggtgacagtgccgggaggtcaggtgcgcgcggtggtgacagtgccgggaggtcaggtgcgcgcggtggtgacagtgccgggaggtcaggtgcgcgcggtcgtgacagtgccgggaggtcaggtgcgcctggtgatgacagttccgggaggtcaggtgcgcgcggtgatgacagtgccgggaggtcaggtgcgcgcggtcgtgacagtgccgggaggtcaggtgcgcgcggtggtgacagttccgggaggtcaggtgcatgcggtgattgttccggaaggtcaagtgcatgtggtcatagttccagaaggtgtccccggggcaggatatgttttcctattttccttggcttccggggagcttggagcctgctttcctttgggtcctgggctgattttcgggcaggtgctgggatgcagtgtcctgggtgctgcagcatggctgagcctcggattctctgtccagggctagctgtgttcaggccgcgcatctttagttttggggcatcgcaaatcctcctggaatctgatgaaagctgtggaccctctgcttgtggcggggggtcatgcagaaaactttgagtacagtttcaggagatgcacggaccacccaagggcctggaatcggagcccccacaagacagggggtgtgctctctgggccagcgtttggagcactctctggctcttggagccctggtggggcattcccagcagcttgcttcatttcctggacacgtgttcttggctgtggtttatattactctctgtatgtaaatgtcataggttttttttcttctagtaaaattgttcctagttagcacagtggaatcttgaaacaagaaagccatctttcatgaacagtgtgtggtttccaggcttagcccatggagttggagtgacaggtatggcagagatggggggtcaggctgcgtagaaggtgtttgcgccccctaggtgcaggtgttaatcctcaccaccagggcgatggtgctaggatgtggggcctttgggaggtgatggggctgaggactgagcctcatagtggggattagtgccctgatgaaagaggcctcagagagcaccctcgcttcttctaccatgtgagcacccagcaggaaggtgctgtccaggacccagaaagtgggtcctctgcagacaccaaatctgctggcaccttgatcttggacttccagcctccggaactgggggaaagcaatgtctgctattgatgagccgcccaagctgcggtgctttgttgcagccccaacaggctaagtcaggaaccgtgaacaggagacctcccagggagtctgctcggaggggggatttgtcgtgggggaattggtcattgtgcctcagtttccccattgttggggaagtggtgcccatggccactggcaggcccccccgggggccagcacaccaggtaggaagaaatgcaagccccaggctgctgagatgcacagagccccacattctgccttcgggacgtctgtctagtcccttgtcttacaaagggtgggcaggtcccctgcaggactcagcagagggagagacacagtcagggagcggccagaggggtgacgtgcccggctgcccacaggtacaatggctccttcgtgtcacacatgggtgtcggcgtcatctttcggctgggtgagggaactgcccagctgcccaagccctgggttcccaagtcagagttgctgggattactgggcagggaggtggtcccgcaacccacaccggggcagtggcaggaagcacagacacacacagcacagcgggccggcaccgccggggtgggttgtctgggcttggactctttctgtgcacatgctgatgtttgttgataataagaacagaaacattttctttttttttttttgagatggagtcttgctctgtcacccaggctggagtgcaatggcgtgatctccgctcactgcaacttccgcctcccaggttcaagcaattctccttcctcagcctcctgagtagctgggattacaggcacgtgccacaacacccggctaatttttgtaatttttagtagagacggagtttcaccatgttgtgcaggctggtcttgaactcctgacctcatgatctgcccgcctcagcctcccaaagtgctgggattacaggcgtgagccactgtgcccagcccctccccttttttttttccttgagacagagtcttgctctattacccaagctggagtgcagtgacgcgatctcagctcactgcaacctccacctcccagattcaagcaatcctcctgcctcagtgtcccgagtagctgggaccacagatgcgtgccatcacacccagctaattttgtatttttggcagagacaggcttttgccatgttgcccaggctggtcgtgaacgcctgagctcaagcgatcttcctgccttggcctcccaaagtgctgggattacaggcgtgagccaccatgccctgcccagaggtgttttcaagcatgtcctgtgcactgggcctggttggttgatgctcagtgatgtttattgaatgaatgaatgaatgaacgaacaagatccagaagtctgagggtaaacatggggtgccagcatcttgtcttagcccagcccgttgttggggggttggcagagtacctcaacccagacccctgtcacctgtgccccctgccccccctcacgctcctccctgtctcccccaggacctcccactctcgggcaaaggccgaggcagccctcacagcagctcagaaagcccaggaggaggcgcggatcgccaggatcactgccaaagagttctccccttccttccagcaccgggaaaacggtgagtctcgccgggcctgatactggcatcgtggggagggggtgcgtggatggctgggcagtcctggcagcagatgtgtcctccagagcgggtaggcttagatgggctcagccccagctgctcccctgcccggtgtcttcctctcccgccgaaggtgcttgtttgtgccaaggcctggcctggctccccgggacacaggacatgtgtcttggcaggtctctcccattcccacagtccttggggcaccgcgtgttgcttgccggctgtccctgacggtgaattcccacctgcccagcctgtgctgtctgctgcccgccctcccccagtgttgttctagaaccttctgggatgatggaactatctgctgggtccagcgtggcagccattagctgggtgtgtctgcggtcctggctcttgacctgcctctgatgtgaactaacttaccattaaacacacccagtggctgctgggttggacagcgtcacaaatggcagctcccgctgtgcccctcggaaagagctggaagctgctgcctgatttaggaacctcgtcctcccttcgagggggggatcatgggtgatacggcccagtgacagtggcgggggcgtggagggtgtggggtgggtggcggagaggccgtggccagctgcctcccatctcctacctcatcttggaggaagcttcctctgcaccaggaggccccagagaatggggttaggagcgcgccccctcaacccgagctctgaccggtgagcggtattcggatgaggctctgcgcgctggaggtccgcacctgacaccacagccctgaacttgctcgcaacgcctgccactctggccaccaggacaggatgaggagcccagcctgggggctgcggggctgggcgctcccctgactggaacgcgagaccacatcatctctagaggtgaccgtccgccaccactccaagaccttgccttcactgggcgagtgccgggccctgcagagcctgcagctgcagcctggaggtcaggaggacgagcccccgcacccccaccgccttgggcagcttctattccactgcgtcccctccactctcctggcccctccagacacacgcaccccaaagtgtccaagtgggagggcaatgggccacgtgcccctgtggccattggcatcctccatggtctcgccctcaggccctgagtttccttctccctggggcccctccttgcctcgcccacgaccagctgcctgggctgggaggcggtggggcgcacacttgggggcacacgtgtagcgtgcacgtgcatgtggggagtgggtgcagtgctgcgggatggacgaacggaacagggatgctggagaggaagccagtgccgtcgggggcacctgccagaaccctggctgggccactccacgcagaatgcacgtaggacggcatgggattccacgtaccaaccctggcccatggtcgcctcagctccagccctgcctgcgactcacattccactgcctgcggcctgttttgtttcctctcagagcctcagttccatgtgtgaaaggaaggggaccagacagggaacattgaagcctccatcctgtgccaaatcccccaagagacgcatccaccggaagctcccaacgtgacttatttgggaggagggtctgtgcaggtgtcattatggaacagactttgagacaagatcagcctggattggggtgagtcctagatccacgcggcgtccttagaagagacagaagaggaggagacaggggcggatggagtgatgtcagagatcactggagactccgtggctggagggtggctggagcggagaccctggaaggaagcagcactcccgacacctggatcacggcttctaacttccagaaacacgagtttgccttttgggtgtttctacccccacccccgcgtggtgctttactgctccagtcccaagaaatgggtgcaggctcccccaccctgagaattccagttaagactctggagtcggagatgcttcctggagggctcagtcctggagtcagaggagatgcttcctggagggctcagtcctggagtcagagatacttcctggagggctcagttctggattcagaggagatgcttcccagagggctcagtcctggagtcagagatgcttcctggagggctcagttctggattcagaggagatgcttcccagagggctcagtcctggagtcagagatgcttcctggagggctcaatcctggagtcagagatgtttcccggagggctcagtcctggagtcagagatgcttcctcgagggctcagtcctggagtcagagatgcttcctggagggctcagtcctggacttggagatacttcctggagggctcagtcctggattcagaggagatgcttcccagagggctcagtcctggagtcagagatgcttcctggagggctcagtcctggattcagaggagatgcttcctggagggctcaggtccctgcaggaggggccttgggctgaccttttaagtcctgaagctgcctcctccgacagttcttgtcttcatgatacagatgcgggcccctgtgggggccgctgtgatggagagacggggccgctgctgcctcactggtgggagtccagtcctcgagggtctggccaagttggcctcacctcctgctgctgcaccttggagcctggctcactctgagcacctcggtccgatctgccacctctttgggtggcgccccggagccttctctgagcagcatcttccctggagggggcgttgggaccgtgtgttttttacaagcgctgggactggcatgagatttgatccaagcatttcttgtttccgccttggatgcattcaggatcagtgttcccagaccaggcaccccggggccttctcggctgctggggaggggtggggctgggggaagctgtccaggtccttcctcagtcgtcctcacagccaggagctgaggtcaccaatgacagatgacagaacagagcctccaagaggggaacagggttgccagagcgctcaggcaggatgtggtggaatcaggatttaaaacgaggctctgaagcctgggccatgccggcttctagagaagcaaaccatccagcgcttcggctgcagagagtggctggcgccagcctcttgctgcctgtctgggaggtccctggcagcctcccagcccgagaactctgaccgtcagggttctgcccaaggccacgtgaaggcaggaccatgaaggagcctggccatgcggtgtccagtgcccaggccacttccggtgccatccgcccccgccccagaggcaggaggactgagcgtcggaggctgcaggacgcaggtggcttgaaggcacaggaagaagctgcaggggcttcacgaggacccgccctgcatcctcaccacgaggcttctaggaacgttacctgctcagcagctcattctagctgagggtgccctcctgctggtggtgctgggagtcggggcccctctagcgtggggtctcctgggccagccaggcctgatctcggcagcagtgggtggacgacctggtgggaaaggccggggcgccatcgggggatggggggggccttgatcccagcctgctaggtccatagcgtctaaggcacaggaatgccagccccttggaagtcccatcctggccaaggaggaggtacgatgtgggtgatccgtgggcatgattgggggcctctgatggggccagggctgccctccacacaggaccctcacggggagagaggtggtggggctgggcgtcaggtgggtacgtggatgtcagcacagttctacacagaccttctctggcccattccctgctctgctcgggggacacccaaccagcccagccctgtttctggggagtgaccacagagaagcagtcattgagacctcagagtggcccacccacacccccgtgtgacaggaactcagacttgggagggctggccacccacccagagctacccagctgtgccccaagccccaggccagggccatggcctcgctctgctcagcactgcctcttagaccagcaaggaggaggacacagcagcccagcctggtggcgtcgcagcgtgccccgaatctgcttgctaacgtctctttacaaacggagggactatctggtgccacaccgagggtagccatgtctcccagaccttctcctgccaaaaggtcagactggggtacaggcgtctcccagaccttctcctgccaaaaggtcagactggggtacaggccaggagaatgtccgagcgtgagagactctaagatgggcagtggctcttggtgtccgagctggtctcatgaggtggccgccccgggtagagctctgagacctccgtcctcagagggaagactccccaaattcccacagagctctgcaccattttggctcgggcaggacggggatgctgggggcgggggtcccactgttctgtgctgagcctaggacgctacttgagatgaacgtgtaactgttccccggagggtaagaagcgccgcgttcttgggtttctcctaatggaataaagtcgggcatggctcccgtgtccctgcgttggcaggaaatgagatcccagtcaatgttcttggttctcacctcctgctgggggctcacggagctccggagaagtctcggggctagtcctacaatgctgcccctccctatccaggcctccgggtgggggctgatggggactgagccaccggcagacgacagggactctgctggctgcagagctggcccctgggtcctgtggtgacctccccttccggggagccccgccctaggacttgaagccccagcagcctcgaaaggtggggagggaagtcggctcctgggctctcacgctggccccggcactggctgtttctcagcaagactgtcccagcaccctgagacacagtcccaggcgggtaggggtgttggcggccttgctgtgacagctcggcctccccagacaggtggtcagctagggtgaggggcttcctcttaccctcaggacaggcgagtcagggttggtgggacctgagtccacaccccagcgtcctcacgcactttccgagccttgggtctcccttgtagcctgggagctgctgtccgcccttcctgtggtgcctgggcctagccagtctccctccaagtgtctgtggggtggggaccacgggggtcccacagagggctttgggagccacggtccccactcccctcccttgcctgcagcctttcggtgagtgggaagcgcctctgctgccctgaggttccctctggcgccctggcccggggaccgcggcctcgctgtggaatgtgctgggtaacgccgtctggcgtcgtcttgtgtccccatacagggctggagtaccagaggccgaagcgtcagacctcctgtgacgacatcgaggtgctgtccaccgggacacccctgcagcaggagagccccgagctgtaccgcaagggcaccactccctccgacctgacccccgacgacagccccctgcagagcttccccaccagccccgcggccaccccgccgcccgcgcccgccgccaggaacaaggtcgcccacttctcgaggcaggtgtcggtggacgaggagcggggcggggacatccagatgctcctggagggccgggccggggactgcgcccgcagcagctggggcgaggagcaggccgggggctccaggggtgtccgcagcggtgccctgcgcggcggcctgctcgtggatgacttccgcacccgaggttcgggccgcaagcagcccgggaaccccaagccgcgggagcggcggacggagtcaccccccgtgttcacgtggacttcccaccaccgggccagcaaccacagccccggaggctccaggctgctggagctgcaggaggagaagctgagcaactaccggatggagatgaaacccttgctgaggatggagacgcatccccagaaaagacgctacagcaagggcggcgcctgccggggcttgggggacgaccaccgccccgaggaccggggcttcggggtgcagagactgcggtccaaggcccagaacaaggagaacttcaggccggcctcctccgcggagcccgccgtgcagaaactggcgagcctgcggctgggcggggccgagccccggttgctgcgttgggacttgaccttctccccgccccagaaatccttgcctgtcgctctagagtccgacgaggagaatggggatgagctcaagtccagtacggtgagtgggcggccaccaggctggtcccagtggaggcaacatccacctctctgctgacctggtgtttcctgagggtttccagcaaggtcactgctcccctgttcctctccaggggtggagtagggtgggggtacagccgggagtggtggccctcaggtcgctgggaccctcccctaggaagccaggtggggcaggtgtcatacctctttccgtgagaggctgcccccaggttgtccagaaagcacacgaaactcctggtttccaggcagcatgggatccctgcgctccccgcaacgtgctcaaccccagctgctccaagagtctgtgcccctggtcagtagccttgggccgtgagatgaccgcttgagtgtcctgtgtcctggataggaacccagaggcgaaacagctgggggcaaggctggccctggggctgggagtcaggtctggggttggggggagctgccagtccaggtttctctcacccatggggggtgttggggttggagccggctcccacgtgaggcccgctgtgtgcaactcaccgtcagcctcacccagcaccccccccccaaattcgttcctccagggcaggcagcttggagggaagaacagcgtccccctgggctagactcggcgcgaggctgggggtcccaggctagactcggtgtgcgaggctgggggtcctggtgcctcacacagggctggcctccccttgaagggaccttcactctgcaaatcccaccctgctcccttgaccagtgctgcaggaatgaagggctttgggaatgaagggctgtgggacggtggctacataggatctgcgcacggccaggccctcaccccagcagggctctgctcgctcgtggctgccaggcagtgcgtggtgctgaacagaagtgctgcaagccagtctgcccctcgggtgtgaggacgcaggggggtgtccgcagggggcgctcagggcgtgtgggccgggcaggggggaccattggtaggtttattgtcgacgctgcggcaggggtcggtgtctgtccctgcccctgacctaggggagctgtggttttctcctcctgccagccccacccccagaggggtcaccgcgtgacacccctttgaaggggcacgtcctgccgggcggcaatgcctgtttctgcctctccttggaaccactgcggacgtctgtctgtcctgttgggttcctaagatgcctccccgttccccaaagcccacagagaacagcccctatgctcctgctgggaccaaaataccagctcacacttacaacttgattttccccatcgcagccggagatgtgtgcgtggctgtgcgcgcgcggttattttaggaacagccaagcagtgggcagggtgggaggtgtagggacgggttctatttttagcctgcttaatgcactgacacttttgggtgcttttaaatgaacctccctgggggtcaatgcacaagctggggacaagtgccaagctggcctctgggatgagttagcgacacgcagggtgctacatgctgccctggtcctgcccggtctccagctggggctaatctggaggtcccgtcagctgcagcatcatgtcccacccaccctggaggatgccagctgagctccttggaagtggccaccctggggtacctggtgctggtgcaaacaaggtttccctccctgtctccctccccgtctccctccccacagctttggagctgtgttttctgccaggtctgtgaggggagatggtgtgtagaggcgcggtcaatcctaagcactgttccaggccctgttggtgggaagcaggaggggcccgtgccagccggcatccatgtcaggggacaggtgtcagggcagtcgggtgccaggctgggctgagtgccttcagtgccaccccctcacaggggtacagctctctgcagtgtctcccctgggccaccagcagggtctggggtgctgtcttctggccatccccctccaccattcccaacatgctgcagagctcacacgccagaggccctgggtcccactggccacagatgggcctgggattctagcccacatagggtcccactggccacagatgggcctgggattctggcccgtgtagggccctggcaattcagggtaaagtcccttaggatggaagcacaggttctccctaccgctctactggtgaaggaactctggccctggctgtttccctcttggtgaaatgatggatatgacctgatggtggggtccagggttggaggatggctggggagacagaagcttccctcctgcgggcagccacgccctcgcccacagcgtccgtacctgcttcccggcccagagcacgggctggactgtggcagaagaggtgccactgaccaccccactctgtcctgctaccccactacaggctgactagcatccggcccttcctcctcgctggggtgggggaactttgcaccaggggtggcatcagtgggcagggctgggcccttggctgttctctgccccctgcaggctggcccggcccaaccaagttgtcatggggcccaagactcacaatacacctgcctgaggccacgatgccacggaaagctgggctcagggcagcttgctttctctctggctgagtggaggcccttgctgtgtccacaactccacttccatctgcgcctcaggtccctgataggctgggaggttgcggagggtgtgggcctggtgctgctgtggctgcaggtgccccctaggcagcacctcaccctctgaggaccctcctctctgagctgcatcccaaggacagtttacaaagccctttgctgactggggttcagcttatctgttgagcaaactttttctttaaaaagtttcttggccaggcacggtggctcacacctataatcccagcactttgagaggccgaggcaggtggatcacctgaggttatcagttcgagaccagcctggccaccagggtgaaatcctgtctccacttaaaaatacaaaaattagccgggcatggtggcacacgcctgtaattccagctactcaggaggctgaggcacaagaatttccagaacccgcaggaggcagaggctgctgtgaactgagatcatgccactgcactccagcctgggcaacagagcgagactccatctcaaaagtttctcaagccagtgtctctagggcctgagggctccaatccccagtgtgaggtgaggcgctcgggcctgggagctgatgcccagcatacagtcgggggtgcccctgccaggtgtggtgcagagcacggcgggggtgtgtgagacctgaggatagcgtctggatgtttctctcacgttaacgggaatgagataggctcattggctgctcatccccaaatccaaatggaaaaccgccattcgggccaggcagaggcaagtactcccacccgttgccgctgccgtgttgagcccaccgtgccgagtggtgttggcaggagcctcccgaggaagctgtgccaacgcaggagtggtcagagcctgtccaggccacagacggggctacgatgagcactgctgcccagggcatgttgggggcagctcctggagaccacagggaggggaggtgggcacccaggtgtgggcagaactggaccaggaggaggaggggcagcagcttgctcaaggcacagggcctcagccacagggtggcgggctgggaaggggcgagggtgtggtgggttctgagtgccccatgcaacggctgttttgggccacgtgctcaagcaggcacccagggaagaccagcctaggtgctggcgtgaggcccttcaacatcagctacgatgctgcttcatcaggagcacagcctggggaaagaatgaaactgacccaggagacaaaagggtccccgggccagacggtcaagcaggaggctgcttcgctgccgcgggatgtggcatccaggctaggcaaggggacggcaggctgccatccccagagggagttctccgggcaaggccgtgaccccactggggctctgagtttgcatccttcctgtgtgtcaagagcagagccggccactgccagcccatagctcccatggtgcggccctggagagctgacttcatgcgggatgtctgctgttctttgcgtagctttactgagacagaaagcgtgtactgttcacttcaggtggccacagtgtacaagtcagggattttagtgtattcccagttatacagccatcacccccaattctatctagatcagctccatcaccttaaacctcagggccattgagcaggcacttgctgtcccctcctcccccagctcccggactctgctttctgcctggtgcccagcttctcacgcagcatgcactgctcctgcgctttctctgtggcagccgcggttgcacggcccatgtcttccccgctgtgtgttcatcctccactggacagctggctctgcttccactccgtggctgttgtgaatcctgctgccgggagtgtgtgcacacacaaggttttgagtggaggcttgactttgccctcttagatttaggcctgggagtggaactgctgggtccccggtgttctcatcaattctcttccagtctctgggagaggagccagcagcagctgcttcgttgtgggtggggggaagggggaggcccgttgttcgggctcccctccttaatgcaggcctgagcagccctcacctggtgccacatgagctactcagtcttaacagaccaccccatcatgtctttgaaatttgaccagaggccagacttgggggcttaaaggagtccatggaaaaccagtctgttaacagggagggggaggagagtagccatccctgaggaatgtgcatcctgggaccttcaggacaagccctggtccatctgtgtcgcacagcagcaggatgtctgcgtggtaggaagtggagggctccgggggctctgaacagctccttaccacagtggggtctacatctccttccaggtggctgcgagatgcagggcgtggtgggggtggggagaggcaggggcaagtgtcctctggaagcagactgtgggcaagactggagtgggtgtcccagccattcctgggctggggccaccgtccaggaggcgtccattccctgtgcagcaggtactgtatctgtagggcagcacccacccctgccgtcctgggagcttcccagaggggccacgctccctctccccctgcctggtgtgagcagaacacagcagagtgcaggactgcaagaatcactgcagaagggcgcccccggaaaaggctctgttgtgagacgtgtcctacctggccccggcaagcacagggaaggtggcagcttggtccgagtgcccaggctgtgcggctgaagggggagtctggcacttggggctccggcggaggctgaggacttgtgagccacttcagggaggtggtggcagcagcattcaggaccataacctaaatccaaggcagctcgcaattggacacagtcctccagagccagttggcgtgaggggtttgttgaggaaggtgtggtgagggggggttttgttgaggacactgtgctcacggactttgggagtcctctgagttccagggcctctctgctgggctgcatctgacactggccatggcagccatgcgggcccttctgagattctgaggtcccagttccacagggcaggagaagcctgctgactggcacaaaggccacacgcaccgagacgtttgcttgcagggaggcctgggtggttctctccaaggggaccctgggagagctggtactcagggtgtcggggccacaagggaattccaagggaattccaggaccagcaacctcagacgtacaggcagctggggcttctctcccgcgtctggacttcacggagctcaggttctggtgggcctttggtgtccaagcgtttctaacatcctcccgtacgcttccacccccgagggtctgctagcttggtgaaaagacgtgggtgggaggcgtggtggcccaggcagagccccccgcagctgtcgctcacctcttcccctcgctctcttccagggctcagcgcctatcctggtggtcatggtgatcttgctcaacatcggagtcgccattctgtttattaactttttcatctgatgagatgtcgcggtagcaaaaatagagaaagggtagaaaaaagggacattaaaattaaaagcaaaaccacaagaagggaaagaccgcaactcggacagcccagcgacttccaagtcctctcacagaagaaccacacgattgggtatcactcacagtttgcctttttttctgggtaatgttttttggattttagccaaaattctttgcttgtataacactctgctgtgtggcatggcagaaggaggccagcacgcagcccctccagctccacgtggagacagaagggatcccggcacatcagtggtaacagcggacgttgtcctcgtggtcacacgtcccgtcttgggtgtggatggagggcagcccggggcagagcctcagccccgcggcccctgagtggcagggctgactcccgtcgacacgagcttagaaagtggattcactgctttctctgtctagaacagacgggtgacaagtatgggcaggaggcatggggcagggtggcccaccccagtgggcagtagcctggcctttttctgtgtgagatctgtgctgcacacctgagggagggggagggatcggccacctcctccctgtgagacggatgcaggtccttccctcttctcggcactgcccccggccttccatgagaagccgactccccacaccgagttttaaagcaaagcccttttcttctgctgcccactcactgtgggtcccattcggctgtttcccccaccagaccccagggaagccggggcccactccgatccgcctgggctcagctaagcacggaagccaagggggctgtgccgtggagctgggctcgcgccggggctctgggtgtgtgcgcttggcgtgcagggtggacgcgtggggttccgtgtccccagcagtgagggccctagaggacgccttctcccatggttactgatctccacgggttttcacatctctgtactgtgcctgcctcaacttcccctaacagatatgcatattccttccagatgcctcagtgctacaccacagtgggcctggtcccaggacaggaatgcggttcaaacccagtggcttgaaacttcctgagaaactgtagcatatccagccccctaaaatgtacaatgtaacttgttcagtccaacaaaaacaggttccttatgtttctgccttctccaccagggtcgctccatcacccaaacaaaagaacaaggtttgccaggatgtccgagtgccccctggccctggctctcgtgtgcatggacgtgcctgaggggtccgggcacggccatacgcaggacccctgtgcccggggaggcgctgcagggattccccatccggtcgtcttggggccagcccgtcttatggactctgccttgctttgcttatgtttagctgtttctctgctacctttcgagcagacttctttactacactgcactggattgctatatttttaaccagaaataaactaaagattagagcatgttccagttaaa

SEQ ID NO: 13 - DNA, 2892 bp, Homo sapiens
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaaaaaaaaaaaaaaaaaaaaaaaaaaaa

SEQ ID NO: 14 - DNA, 1472 bp, Homo sapiens
atccttcacctgttgcctggctagagttgtctggctccactttgagctcttgcagaaccagccctttttcgtgtggtccaggaaagtccatgcctggcaccacctcctcctctagtgactccacgtagaagagagtcctggctggctgctgagtgccctgcccaggagccccttgctgcagcctcgtggcaactggaagcagggtgccattcagcggattgaaggaagaggaggaagaggacggggaggacgatgaagaggaagaggaggaaggcttcttccagaaagtgctcacaccgcttctctcttggcttttgagcaggcgactctggctgggtccccagtgctcaaagctgccactgccgtcctgttgcaggcagcctccccccgccgggccgccggtggaaggagacgggtggctgaagagtttccagcggagtcgcagaatgtgcttcacatcgaagtcttttcgcccagagcctgacatgctttacgcacagaaggcaaaaggctggcagctcacgcaggattctggaggctgggaagttcaagaccaatgcacgagaatttggtctaaagagaatcttcttgctctgaacacacatagtagaaggcagaagggcaagagagagaacaaagtctgtgtctccacatggcagaagagcagaggagacagaacctactcctctatggcaaccaccccatcaatgacaaaaatcctagaaggatgtatgtataggaagttgaagtgttgagaagagaatggctcagagtcaagcgggaacaagattcaaacttcagagagagagggaagaaaaacatttaaatatatctggcataatccaagactatttacgacaagtgttctgtgtttctaataataaaacagacttcacctcggagtacctgcagaactgggaccccaatgaccagggagaatgaagaacaacttgttgaagattgccttttctgactcccagcttccacggagagattaactctgttggctgaagccctattcccaattccttggctagaccctgggtccttcatgttagaaaacctggctttactactactactactactactactactactactgctgctgctgctgctgctgctgctgctgctgctgctgctgctgcattttttaaaaatatattatcttattttactatttgatgttataattgttatatatttttccacacttcctcatactgcttatctcttacttaagaatttatgaataaagaattgatttttcaatacatccttccaaaaattatctgatgttgagttagttgctctctcttgtgcattctcagtcctcacaagcctttctcaaacacaatgtttatcaaagaaaattgtagcaaccaatatacttagtggaatttctcacagagtttgagtgtaggaaacagtattcactgtatattagtcattttgctcccaatagaaggtgc

SEQ ID NO: 15 - DNA, 3997 bp, Homo sapiens
gtctccagcgggagcgcgagacgctggtcaggctccgcggcgcagctcgaaaaggaataatcgcccccgattgactgaaattcctccggagccggcgccgcggccgcccgcgcccgagaccgcgctccggggccgcgtcctcctctcctccggaaaacgctcgcgacccagggccgccggcggccgcgactctgctgtgtcgatcgcctgagtccgttttcaccgtttgcgggatctggaaccgagttacatgcatgtccagtgggggcaggtttaattttgacgacggagggtcctactgtggaggctgggaggacggcaaggcgcacggccatggcgtctgcaccggccccaagggccaaggcgaatacaccggctcgtggagccacggcttcgaggtgctgggcgtctacacctggcccagcggcaacacgtaccagggcacctgggcgcagggcaagcgccacggcatcggcctggagagcaaggggaagtgggtgtacaagggcgagtggacgcacggattcaaggggcgctacggggtgcgggagtgcgcgggcaacggggccaaatacgaagggacctggagcaacgggctgcaggacggctacgggaccgagacctactcggacggagggacctaccagggccagtgggtcggtggcatgcgccagggctacggcgtccggcagagcgtcccgtatggcatggccgcggtcatccgctcacccctgaggacgtccatcaactccctgcgcagcgagcacaccaacggcacggcgctgcatcccgacgcctctccggcggtggccggcagcccggccgtgtcccgcgggggcttcgtgctcgtggcccacagtgactccgagatcctcaagagcaagaagaaggggctgtttcggcgctcgctgctgagtgggctgaagctgcgcaagtcggagtccaagagcagcctggccagccaacgcagcaagcagagctcctttcgcagcgaggcgggcatgagcaccgtcagctccacggccagcgacatccactccaccatcagcctgggcgaggctgaggccgagctggcggtcatcgaggacgacatcgacgccaccaccaccgagacctacgtgggcgagtggaagaacgacaaacgctccggcttcggcgtgagccagcgctcggacgggctcaagtacgagggcgagtgggccagcaaccggcgccatggctacggctgcatgaccttcccggacggcaccaaggaggagggcaagtacaagcagaacatcctcgtcggcggcaagcgcaagaacctcatccccctgcgggccagcaagatccgcgagaaggtggaccgcgccgttgaggccgctgagcgggccgccaccatcgccaagcagaaggctgagatcgcggcttccaggacctcccactctcgggcaaaggccgaggcagccctcacagcagctcagaaagcccaggaggaggcgcggatcgccaggatcactgccaaagagttctccccttccttccagcaccgggaaaacgggctggagtaccagaggccgaagcgtcagacctcctgtgacgacatcgaggtgctgtccaccgggacacccctgcagcaggagagccccgagctgtaccgcaagggcaccactccctccgacctgacccccgacgacagccccctgcagagcttccccaccagccccgcggccaccccgccgcccgcgcccgccgccaggaacaaggtcgcccacttctcgaggcaggtgtcggtggacgaggagcggggcggggacatccagatgctcctggagggccgggccggggactgcgcccgcagcagctggggcgaggagcaggccgggggctccaggggtgtccgcagcggtgccctgcgcggcggcctgctcgtggatgacttccgcacccgaggttcgggccgcaagcagcccgggaaccccaagccgcgggagcggcggacggagtcaccccccgtgttcacgtggacttcccaccaccgggccagcaaccacagccccggaggctccaggctgctggagctgcaggaggagaagctgagcaactaccggatggagatgaaacccttgctgaggatggagacgcatccccagaaaagacgctacagcaagggcggcgcctgccggggcttgggggacgaccaccgccccgaggaccggggcttcggggtgcagagactgcggtccaaggcccagaacaaggagaacttcaggccggcctcctccgcggagcccgccgtgcagaaactggcgagcctgcggctgggcggggccgagccccggttgctgcgttgggacttgaccttctccccgccccagaaatccttgcctgtcgctctagagtccgacgaggagaatggggatgagctcaagtccagtacgggctcagcgcctatcctggtggtcatggtgatcttgctcaacatcggagtcgccattctgtttattaactttttcatctgatgagatgtcgcggtagcaaaaatagagaaagggtagaaaaaagggacattaaaattaaaagcaaaaccacaagaagggaaagaccgcaactcggacagcccagcgacttccaagtcctctcacagaagaaccacacgattgggtatcactcacagtttgcctttttttctgggtaatgttttttggattttagccaaaattctttgcttgtataacactctgctgtgtggcatggcagaaggaggccagcacgcagcccctccagctccacgtggagacagaagggatcccggcacatcagtggtaacagcggacgttgtcctcgtggtcacacgtcccgtcttgggtgtggatggagggcagcccggggcagagcctcagccccgcggcccctgagtggcagggctgactcccgtcgacacgagcttagaaagtggattcactgctttctctgtctagaacagacgggtgacaagtatgggcaggaggcatggggcagggtggcccaccccagtgggcagtagcctggcctttttctgtgtgagatctgtgctgcacacctgagggagggggagggatcggccacctcctccctgtgagacggatgcaggtccttccctcttctcggcactgcccccggccttccatgagaagccgactccccacaccgagttttaaagcaaagcccttttcttctgctgcccactcactgtgggtcccattcggctgtttcccccaccagaccccagggaagccggggcccactccgatccgcctgggctcagctaagcacggaagccaagggggctgtgccgtggagctgggctcgcgccggggctctgggtgtgtgcgcttggcgtgcagggtggacgcgtggggttccgtgtccccagcagtgagggccctagaggacgccttctcccatggttactgatctccacgggttttcacatctctgtactgtgcctgcctcaacttcccctaacagatatgcatattccttccagatgcctcagtgctacaccacagtgggcctggtcccaggacaggaatgcggttcaaacccagtggcttgaaacttcctgagaaactgtagcatatccagccccctaaaatgtacaatgtaacttgttcagtccaacaaaaacaggttccttatgtttctgccttctccaccagggtcgctccatcacccaaacaaaagaacaaggtttgccaggatgtccgagtgccccctggccctggctctcgtgtgcatggacgtgcctgaggggtccgggcacggccatacgcaggacccctgtgcccggggaggcgctgcagggattccccatccggtcgtcttggggccagcccgtcttatggactctgccttgctttgcttatgtttagctgtttctctgctacctttcgagcagacttctttactacactgcactggattgctatatttttaaccagaaataaactaaagattagagcatgttccagttaaa

SEQ ID NO: 16 - RNA, 21 nt, Artificial
  Oligonucleotide
nagnagnagnagnagnagnag

SEQ ID NO: 17 - RNA, 25 nt, Artificial
  Oligonucleotide
cagcagcagcagcagcagcagnnnn

SEQ ID NO: 18 - RNA, 15 nt, Artificial
  Oligonucleotide
cagcagcagcagcag

SEQ ID NO: 19 - RNA, 15 nt, Artificial
  Oligonucleotide
nagnagnagnagnag

SEQ ID NO: 20 - RNA, 15 nt, Artificial
  Oligonucleotide
cngcngcngcngcng

SEQ ID NO: 21 - RNA, 21 nt, Artificial
  Oligonucleotide
cagaggaccaccagaccaagg

SEQ ID NO: 22 - DNA, 23 bp, Artificial
  Primer
gctcatggtcctcaagatctcac

SEQ ID NO: 23 - DNA, 20 bp, Artificial
  Primer
gggtcagtgcctcagctttg

SEQ ID NO: 24 - DNA, 21 bp, Artificial
  Primer
gtgtgagtcgctccagaaacg

SEQ ID NO: 25 - DNA, 23 bp, Artificial
  Primer
ccaccacaggaccatgttatttc

SEQ ID NO: 26 - DNA, 22 bp, Artificial
  Primer
ggaatacctcacactcaaggcc

SEQ ID NO: 27 - DNA, 25 bp, Artificial
  Primer
cacggaacacaaaggcactgaatgt